(19) United States
(12) Patent Application Publication
(10) Pub. No.: US 2025/0057886 A1
(43) Pub. Date: Feb. 20, 2025

(54) METHODS AND COMPOSITIONS FOR TREATING HEARING LOSS

(71) Applicant: Lineage Cell Therapeutics, Inc., Carlsbad, CA (US)

(72) Inventors: Olga MIZRAHI, Jerusalem (IL); Rami SKALITER, Ness Ziona (IL); Francois BINETTE, Napa, CA (US); Dana HAYOUN NEEMAN, Jerusalem (IL); Ofer WISER, Jerusalem (IL); Lilach ALON, Jerusalem (IL)

(21) Appl. No.: 18/842,871

(22) PCT Filed: Mar. 2, 2023
(86) PCT No.: PCT/US2023/014357

§ 371 Date: Aug. 30, 2024

Related U.S. Application Data
(60) Provisional application No. 63/322,110, filed on Mar. 21, 2022.
(60) Provisional application No. 63/315,830, filed on Mar. 2, 2022.

(51) Int. Cl. A61K 35/36; A61K 9/00; C12N 5/0793

(52) U.S. Cl. CPC A61K 35/36; A61K 9/0046; C12N 5/062; C12N 2501/105; C12N 2501/115; C12N 2501/13; C12N 2501/155; C12N 2506/02

(57) ABSTRACT

Provided herein are methods for inducing cellular differentiation of undifferentiated stem cells into cells capable of functioning as sensory cells of the ear, and to pharmaceutical compositions for treating auditory conditions in a subject.

### RELATED APPLICATIONS

This application claims priority to, and the benefit of, U.S. Provisional Application No. 63/315,830, filed on Mar. 2, 2022 and 63/322,110, filed Mar. 21, 2022. The contents of each of the aforementioned patent applications are incorporated herein by reference in their entireties.

### FIELD OF TECHNOLOGY

This invention generally relates to compositions and methods for inducing cellular differentiation of pluripotent stem cells into cells capable of functioning as auditory cells of the ear, and to methods of treatment that employ such auditory cells for the treatment of auditory conditions in a subject.

### BACKGROUND

More than 5% of the population in industrialized nations have significant auditory or hearing loss conditions that range in severity from modest difficulty with speech comprehension to profound deafness. Hearing loss is age-related, as about 4% of people under 45 years old and about 34% of those over 65 years old have debilitating hearing loss. In most cases, the cause is related to degeneration and death of hair cells and their associated spiral ganglion neurons.

The ear is composed of four main sections: the external ear, middle ear, inner ear, and the transmission pathway to the hearing center in the brain. The inner ear is a capsule of very dense bone containing a fluid that communicates with the middle ear. Small bones within the middle ear (the malleus, incus, and stapes) transmit sound energy from the tympanic membrane to the oval window at the entrance to the cochlea of the inner ear. The action of the stapes at the oval window exerts pressure on the fluid within the cochlea. The pressure is transmitted through the cochlea, ultimately causing a second window, the round window to oscillate. A basilar membrane that defines the fluid-filled chambers of the cochlea then transmits the oscillations to the organ of Corti. Hair cells are located in the epithelial lining of the inner ear (i.e., in the cochlear organ of Corti), as well as in the vestibular sensory epithelia of the saccular macula, the utricular macula, and the cristae of the three semicircular canals of the labyrinth. The cochlear hair cells send signals to the cochlear spiral ganglion, and the clustered neuronal cell bodies convey those signals to the cochlear nucleus of the brain stem.

Mechanosensitive sensory hair cells are the basis of our senses of hearing and balance. Our inner ear harbors about 13,000-15,000 cochlear and about the same number of vestibular sensory hair cells, which are the mechanoreceptors of our senses of hearing and balance. Because of their paucity, molecular studies on hair cells have been limited, and consequently the molecular basis of their function is unknown. Aside from being scarce, hair cells are also sensitive to mechanical and chemical insults. Acoustical overstimulation, chemotherapy, aminoglycoside drug side effects, the effects of aging, and increasingly noisy environments contribute to the deterioration of hearing over time. As a result, hundreds of millions of patients worldwide are permanently debilitated by hearing loss and balance problems. The main reason for the permanence of these chronic disorders is the fact that mammalian cochlear hair cells do not spontaneously regenerate and that the limited regeneration observed in the vestibular system is inadequate to restore function.

Auditory neuropathy is a hearing disorder in which the inner ear successfully detects sound but has a problem with sending signals from the ear to the brain. Current state of the art medical knowledge suggests that auditory neuropathies play a substantial role in hearing impairments and deafness. Hearing depends on a series of complex steps that change sound waves in the air into electrical signals. The auditory nerve then carries these signals to the brain. Outer hair cells help amplify sound vibrations entering the inner ear from the middle ear. When hearing is working normally, the inner hair cells convert these vibrations into electrical signals that travel as nerve impulses to the brain, where the brain interprets the impulses as sound. Auditory neuropathy can be caused by a number of factors including: (i) damage to the auditory neurons that transmit sound information from the inner hair cells-specialized sensory cells in the inner ear—to the brain; (ii) damage to the inner hair cells themselves; (iii) inherited genes with mutations or suffering damage to the auditory system, either of which may result in faulty connections between the inner hair cells and the auditory nerve, which leads from the inner ear to the brain; or (iv) damage to the auditory nerve itself. Researchers are still seeking effective treatments for those affected with auditory neuropathy.

Several protocols have been developed for differentiation of human pluripotent stem cells, such as human embryonic stem cells (hESCs) and induced pluripotent stem cells (iPSCs) into sensory cells of the ear that can be used in cellular therapy for treating hearing loss. While these methods have been successful in generating sensory cells of the ear, challenges remain with respect to quality, scalability, and cost of goods associated with translating the existing protocols to a clinical commercial-scale production process of such sensory cells.

Accordingly, there is a need for improved methods for differentiating pluripotent stem cells into sensory cell populations of the ear, and compositions comprising these differentiated pluripotent stems. Such methods should be easily scalable to produce sufficient quantities of sensory cells of the ear for cell therapy applications while consistently and reproducibly producing the targeted sensory cells and pharmaceutical formulations thereof with the desired sensory cell quality attributes for the treatment of auditory conditions.

### SUMMARY

The disclosure provides pharmaceutical compositions, comprising a population of auditory cells. In some embodiments, (a) greater than or equal to 20% of the cells in the population express SOX2; (b) greater than or equal to 10% of the cells in the population express β tubulin III; (c) greater than or equal to 5% of the cells in the population express TrkB; and (d) less than or equal to 1% of the cells in the population express TRA-1-60 and/or SSEA5. In some embodiments, the pharmaceutical composition comprises a population of at least 100,000 cells. In some the population of cells comprises between 100,000 cells and 10 million cells. In some embodiments, the pharmaceutical composition comprises a pharmaceutically acceptable carrier.

In some embodiments of the pharmaceutical compositions of the disclosure, greater than or equal to 30% of the cells in the population express SOX2. In some embodiments, greater than or equal to 30% of the cells in the population express β tubulin III. In some embodiments, greater than or equal to 20% of the cells in the population express TrkB. In some embodiments, less than or equal to 0.1% of the cells in the population express TRA-1-60 and/or SSEA5. In some embodiments, greater than or equal to 50% of the cells in the population express Nestin. In some embodiments, greater than or equal to 30% of the cells in the population express PAX2. In some embodiments, less than or equal to 60% of the cells in the population express PAX8. In some embodiments, greater than or equal to 10% of the cells in the population express GluA4. In some embodiments, less than or equal to 40% of the cells in the population express Myo7A. In some embodiments of the pharmaceutical compositions of the disclosure, greater than or equal to 50% of the cells in the population express CD133.

In some embodiments of the pharmaceutical compositions of the disclosure, (a) greater than or equal to 30% of the cells in the population express SOX2; (b) greater than or equal to 30% of the cells in the population express PAX2; (c) greater than or equal to 30% of the cells in the population express β tubulin III; (d) greater than or equal to 20% of the cells in the population express TrkB; (e) greater than or equal to 30% of the cells in the population express GluA4; (f) less than or equal to 20% of the cells in the population express Myo7A; and (d) less than or equal to 0.1% of the cells in the population express TRA-1-60 and/or SSEA5.

In some embodiments of the pharmaceutical compositions of the disclosure, (a) between about 30% to 95% of the cells in the population express SOX2; (b) between about 10% to 60% of the cells in the population express B tubulin III; (c) between about 5% to 70% of the cells in the population express TrkB; and (d) between 0 to about 1% of the cells in the population express TRA-1-60 and/or SSEA5. In some embodiments, between about 30% to 95% of the cells in the population express PAX2. In some embodiments, between about 5% to 95% of the cells in the population express GluA4. In some embodiments, between 0 to about 30% of cells in the population express Myo7A.

In some embodiments of the pharmaceutical compositions of the disclosure, the population of auditory cells comprises non-neuronal ectoderm (NNE) cells, pre-placodal ectoderm (PPE) cells, early otic neuronal progenitor (ONP) cells, mid ONP cells, late ONP cells, or any combination thereof. In some embodiments, the population of auditory cells comprises sensory cell populations of the ear. In some embodiments, the sensory cell populations are selected from the group consisting of hair cells, supporting cells, otic neuronal progenitor cells and sensory neuronal progenitor cells.

In some embodiments of the pharmaceutical compositions of the disclosure, the composition comprises a cryopreservation medium.

In some embodiments of the pharmaceutical compositions of the disclosure, the composition comprises aggregates of cells, single cells, or a combination thereof.

In various embodiments, methods for obtaining a population of auditory cells derived from undifferentiated pluripotent stem cells are provided.

The disclosure provides methods for obtaining a population of auditory cells derived from undifferentiated pluripotent stem cells comprises: a) obtaining a culture of pluripotent stem cells; b) culturing the pluripotent cells for a first time period under culture conditions sufficient to induce differentiation of the pluripotent cells to non-neuronal ectoderm cells; and c) culturing the non-neuronal ectoderm cells from b) under culture conditions sufficient to differentiate the non-neuronal ectoderm cells to auditory cells.

The disclosure provides methods for producing a composition comprising a population of auditory cells, the method comprising (a) culturing a population of undifferentiated pluripotent stem cells in a first cell culture medium comprising Bone morphogenetic protein 4 (BMP4), Fibroblast growth factor 2 (FGF2), and 4-[4-(2H-1,3-Benzodioxol-5-yl)-5-(pyridin-2-yl)-1H-imidazol-2-yl]benzamide (SB431542) for 1-9 days under conditions sufficient to produce non-neuronal ectodermal (NNE) cells, thereby producing a population of cells comprising NNE cells; (b) culturing the population of cells comprising NNE cells produced in step (a) in a second cell culture medium comprising SB431542, FGF2, and N-(6-Methyl-2-benzothiazolyl)-2-[(3,4,6,7-tetrahydro-4-oxo-3-phenylthieno[3,2-d]pyrimidin-2-yl)thio]-acetamide (IWP-2) and 4-{6-[4-(Piperazin-1-yl)phenyl]pyrazolo[1,5-a]pyrimidin-3-yl}quinoline (LDN193189) for 1-9 days under conditions sufficient to produce pre-placodal ectodermal (PPE) cells, thereby producing a population of cells comprising PPE cells; (c) culturing the population of cells comprising PPE cells produced in step (b) in a third cell culture medium comprising 6-((2-((4-(2,4-Dichlorophenyl)-5-(4-methyl-1H-imidazol-2-yl)pyrimidin-2-yl)amino)ethyl)amino) nicotinonitrile (CHIR99021), FGF2, and Insulin-like growth factor 1 (IGF-1) for 5-9 days under conditions sufficient to produce early otic neuronal progenitor (ONP) cells, thereby producing a population of cells comprising early ONP cells; (d) culturing the population of cells comprising early ONP cells produced in step (c) in a fourth cell culture medium comprising Sonic Hedgehog (SHH), retinoic acid (RA), Epidermal growth factor (EGF), FGF2 and IGF-1 for 5-9 days under conditions sufficient to produce mid-late ONP cells, thereby producing a population of cells comprising mid-late ONP cells; (e) culturing the population of cells comprising mid-late ONP cells produced in step (d) in a fifth cell culture medium comprising Brain derived neurotrophic factor (BDNF), Neurotrophin-3 (NT3), and IGF-1 for 3-65 days under conditions sufficient to produce late ONP cells, thereby producing a population of cells comprising late ONP cells; and (f) collecting the population of cells, thereby producing the composition comprising the population of auditory cells. In some embodiments, the first cell culture medium does not comprise FGF2.

In some embodiments, the BMP4 is at a concentration of between about 1-25 ng/ml in the first cell culture medium. In some embodiments, the BMP4 is at concentration of 10 ng/mL in the first cell culture medium. In some embodiments, the SB431542 is at a concentration of between about 0.1-10 μM in the first and/or second cell culture medium. In some embodiments, the SB431542 is at a concentration of about 1 μM in the first and/or second cell culture medium. In some embodiments, the FGF2 is at a concentration of between about 1-25 ng/ml in the first, second, third and/or fourth cell culture medium. In some embodiments, the FGF2 is at a concentration of 10 ng/mL in the first, second, third and/or fourth cell culture medium. In some embodiments, the IWP-2 is at a concentration of between about 0.5-10 μM in the second cell culture medium. In some embodiments, the IWP-2 is at a concentration of 2 μM in the second cell culture medium. In some embodiments, the LDN193189 is at a concentration of between about 20-400 nM in the second cell culture medium. In some embodiments, the LDN193189 is at a concentration of 100 nM in the second cell culture medium. In some embodiments, the CHIR99021 is at a concentration of between about 1-25 μM in the third cell culture medium. In some embodiments, the CHIR99021 is at a concentration of 6 μM in the third cell culture medium. In some embodiments, the IGF-1 is at a concentration of between about 5-100 ng/ml in the third, fourth and/or fifth cell culture medium. In some embodiments, the IGF-1 is at a concentration of 50 ng/ml in the third, fourth and/or fifth cell culture medium. In some embodiments, the SHH is at a concentration of between about 50-1000 ng/mL in the fourth cell culture medium. In some embodiments, the SHH is at a concentration of 500 ng/ml in the fourth cell culture medium.

In some embodiments, the RA is at a concentration of between about 0.2-2 μM in the fourth cell culture medium. In some embodiments, the RA is at a concentration of 0.5 μM in the fourth cell culture medium. In some embodiments, the EGF is at a concentration of between about 5-100 ng/ml in the fourth cell culture medium. In some embodiments, the EGF is at a concentration of 20 ng/ml in the fourth cell culture medium. In some embodiments, the BDNF is at a concentration of between about 5-100 ng/ml in the fifth cell culture medium. In some embodiments, the BDNF is at a concentration of 10 ng/ml in the fifth cell culture medium. In some embodiments, the NT3 is at a concentration of between about 5 and 100 ng/ml in the fifth cell culture medium. In some embodiments, the NT3 is at a concentration is at a concentration of 10 ng/ml in the fifth cell culture medium. In some embodiments, the undifferentiated pluripotent stem cells comprise human embryonic stem cells (hESCs) or human induced pluripotent stem cells (hiPSCs).

In some embodiments, culture of undifferentiated pluripotent stem cells and/or auditory cells comprises dynamic culture conditions.

In some embodiments, the auditory cells are grown in static two-dimensional (2D) adherent culture either is small scale platform such as tissue culture flask or large scale platform such as multi-layered flasks.

In some embodiments, the methods comprise dynamic culture conditions at any one of steps (a)-(e).

In some embodiments, the auditory cells are grown in static three-dimensional (3D) culture such as cell aggregates on non-adherent substrate.

In some embodiments, the auditory cells are grown in dynamic two-dimensional (2D) large scale platform such as microcarriers suspended in a bioreactor.

In some embodiments, the auditory cells are grown in dynamic three-dimensional (3D) large scale platform such as aggregates suspended in a bioreactor.

In some embodiments, the suspension of auditory cells comprises aggregates of auditory cells.

In some embodiments, the methods comprise, prior to step (a), seeding the undifferentiated pluripotent stem cells at a density of 1,200-20,000 live cells/cm2 in a monolayer, and culturing the cells to until a lactate concentration in the cell culture medium is 1.5-12.5 mM, and a percent confluency is 5-80%.

In some embodiments, the population of undifferentiated pluripotent stem cells are cultured in the first cell culture medium for 3-7 days. In some embodiments, the population of cells comprising NNE cells are cultured in the second cell culture medium for 3-7 days. In some embodiments, the population of cells comprising PPE cells are cultured in the third cell culture medium for 7 days. In some embodiments, the population of cells comprising early ONP cells are cultured in the fourth cell culture medium for 7 days. In some embodiments, culturing the population of cells comprising mid-late ONP cells comprises: (i) harvesting the population of cells comprising mid-late ONP cells; (ii) seeding the population of harvested cells in containers comprising the fifth cell culture medium; (iii) culturing the population of seeded cells for between 7 and 35 days; (iv) harvesting the population of cells; (v) seeding the population of cells in containers comprising the fifth cell culture medium; and (vi) culturing the population of cells of 7 to 30 days.

In some embodiments, the methods comprise, prior to step (e), cryopreserving the population of cells comprising mid-late ONP cells, followed by thawing and culturing in the fifth cell culture medium.

In some embodiments, the methods comprise comprising cryopreserving the population of auditory cells.

In some embodiments, the method for obtaining a population of auditory cells comprises obtaining a culture of pluripotent stem cells, culturing the pluripotent stem cells for a first time period under dynamic 2D or dynamic 3D culture conditions sufficient to induce differentiation of the hESCs to non-neuronal ectoderm cells and culturing the non-neuronal ectoderm cells under culture conditions sufficient to differentiate the non-neuronal ectoderm cells to auditory cells.

In some embodiments, the method for obtaining a population of auditory cells comprises obtaining a dynamic culture of undifferentiated hESCs, culturing the undifferentiated hESCs under culture conditions sufficient to induce differentiation of the hESCs into mid-late ONP cells, e.g., using the combination of cell culture media described herein, and formulating a cryopreserved mid-late ONP cell composition for generating intermediate cells bank. In some embodiments, the methods comprise characterizing and releasing thawed cells from the bank for further culturing the mid-late ONP cells under dynamic culture conditions sufficient to differentiate the mid-late ONP cells to auditory cells.

In some embodiments, the method for obtaining a population of auditory cells comprises obtaining a dynamic culture of undifferentiated hESCs, culturing the undifferentiated hESCs for a first time period under conditions sufficient to induce differentiation of the hESCs to non-neuronal ectoderm cells culturing the non-neuronal ectoderm cells under culture conditions sufficient to differentiate the non-neuronal ectoderm cells to pre-placodal ectoderm cells, culturing the pre-placodal ectoderm cells under culture conditions sufficient to differentiate the pre-placodal ectoderm cells to early otic neural progenitors, culturing the early otic neural progenitors under culture conditions sufficient to differentiate the early otic neural progenitors to mid otic neural progenitors, culturing the mid otic neural progenitors under culture conditions sufficient to differentiate the mid otic neural progenitors late otic progenitors, and formulating a cryopreserved cell composition with same for generating intermediate cells bank of Mid-late otic neural progenitors, a method to characterize and release thawed cells from the bank for further culturing the cells under dynamic culture conditions sufficient to differentiate the cells to, for example, sensory neuronal progenitor cells such as spiral ganglion calls.

The disclosure provides methods of formulating a cryopreserved auditory cell composition, for administration to a subject directly after thawing is provided.

In some embodiments, the method of formulating a cryopreserved auditory cell composition comprises (a) suspending the auditory cells in a cryopreservation media to form a cell suspension, (b) storing the cell suspension at a cryopreservation temperature (less than or equal to −80° C., or less than or equal to −140° C.), and (c) thawing the cryopreserved suspension for administration to a subject. In embodiments, the cryopreserved auditory cell composition is formulated for long term storage for re-seeding for the continuation of the process directly after thawing. In embodiments, the cryopreserved auditory cell composition is formulated for administration to a subject directly after thawing.

The disclosure provides methods of treating a subject with an auditory condition, comprising administering a therapeutically amount of the pharmaceutical compositions of the disclosure to an inner or middle ear of the subject.

The disclosure provides methods of treating a subject with an auditory condition, comprising administering a therapeutically effective amount of a pharmaceutical composition comprising a population of auditory cells, wherein (a) greater than or equal to 20% of the cells in the population express SOX2; (b) greater than or equal to 10% of the cells in the population express β tubulin III; (c) greater than or equal to 5% of the cells in the population express TrkB; and (d) less than or equal to 1% of the cells in the population express TRA-1-60 and/or SSEA5 wherein the composition is administered to the inner or middle ear of the subject.

In some embodiments of the methods of treatment of the disclosure, (a) greater than or equal to 30% of the cells in the population express SOX2; (b) greater than or equal to 30% of the cells in the population express PAX2; (c) greater than or equal to 30% of the cells in the population express β tubulin III; (d) greater than or equal to 20% of the cells in the population express TrkB; (e) greater than or equal to 30% of the cells in the population express GluA4; (f) less than or equal to 20% of the cells in the population express Myo7A; and (d) less than or equal to 0.1% of the cells in the population express TRA-1-60 and/or SSEA5.

In some embodiments, the composition is administered via injection. In some embodiments, the injection comprises administration a Scala tympani or modiolus of the subject. In some embodiments, the injection comprising inserting a cannula through a hole in the otic capsule, or inserting a cannula through the round window. In some embodiments, between about 100K to 1 million cells are administered to the subject.

The disclosure provides compositions for use in the treatment of any auditory condition in a subject, comprising the pharmaceutical compositions comprising populations auditory cells described herein.

The disclosure provides compositions for use in the manufacture of a medicament for the treatment of any auditory condition in a subject, comprising the pharmaceutical compositions comprising populations auditory cells described herein.

The disclosure provides kits comprising the pharmaceutical compositions described herein.

n some embodiments, a pharmaceutical composition for administration to a subject is provided. The pharmaceutical composition comprises the auditory cells as described herein and a cryopreservation media.

In embodiments, a pharmaceutical composition for administration to a subject is provided. The pharmaceutical composition comprises the auditory cells as described herein and a cryopreservation media.

In some embodiments, the auditory hearing condition is conductive hearing loss, sensorineural hearing loss, central hearing loss, mixed hearing loss, auditory neuropathy spectrum disorder, central auditory processing disorder, or tinnitus.

In some embodiments, the pharmaceutical composition is administered to the inner ear.

In some embodiments, the pharmaceutical composition is administered to the middle ear.

In some embodiments, the present disclosure provides a method for replacing auditory neurons in a subject in need thereof, the method comprising administering to said subject a therapeutically effective amount of any of the pharmaceutical compositions described herein.

In some embodiments, the present disclosure provides a method for augmenting an existing but damaged auditory neuron population in a subject in need thereof, the method comprising administering to said subject a therapeutically effective amount of any of the pharmaceutical compositions described herein.

### BRIEF DESCRIPTION OF THE DRAWINGS

FIG. 1A shows the stages of auditory neuron (AN) differentiation for an exemplary method of the disclosure, including a scheme for directed differentiation of human embryonic stems cells (hESCs) toward placode-derived spiral ganglion-like sensory neurons. Included are exemplary differentiation time frames (DTF) along the process.

FIG. 1B shows the growth factors in different stages of auditory neuron differentiation for an exemplary method of the disclosure. hESCs are exposed to different combinations of growth factors for directed differentiation toward otic neuronal progenitor cells. The differentiating factors used in each DTF are shown.

FIG. 2 shows images of auditory neuron (AN) morphology at the end of each DTF as described in Example 1.

FIG. 3 shows images of human embryonic stem cell (hESC) morphology prior to differentiation initiation, as described in Example 2.

FIG. 4 shows images of DTF #1 cellular morphology, as the cells differentiate towards ANs, as detailed in Table 10 and Example 3.

FIG. 5 shows images of DTF #2 cellular morphology, as the cells differentiate towards ANs, as detailed in Table 12 and Example 4.

FIG. 6 shows images of the morphology of cells exposed to different growth factor (GF) combinations in DTF #1, as described in Table 14 and Example 5.

FIG. 7 shows images of the morphology of cells reseeded in different culture systems (Static, Dynamic, Single cells, Aggregates) during Otic Neuronal Progenitor (ONP) maturation, as described in Table 16 and Example 6.

FIG. 8 shows images of the morphology of thawed and ongoing cultured aggregates in different culturing systems (Static, Dynamic) as detailed in Table 17 and Example 7.

FIG. 9A shows an exemplary scheme for directed differentiation of human embryonic stems cells toward placode-derived spiral ganglion-like sensory neurons.

FIG. 9B shows a scheme for directed differentiation of human embryonic stems cells toward otic neuronal progenitor cells.

FIG. 9C shows an exemplary cell culture scheme and protocol for directed differentiation of human embryonic stems cells toward otic neuronal progenitor cells in accordance with the present disclosure. hESCs are cultured for 0 to 3 days using iMatrix-511 direct coated vessels (one step inoculation). Culturing and differentiating cells may also proceed on direct coated MCs (one step inoculation) in PB wheel bioreactors. Differentiation may continue for 3 to 32 days, optionally replacing SB431542 (TGF-Beta inhibitor) with NIC according to Needham and Nayagam 2014. Production of ONPs can be done through neural crest induction (using FGF and EGF only)-possibly combined with FBi protocol (inhibition of FGF and BMP signaling). On day 26, the cells may be cryopreserved to create an intermediate cell bank (ICB, LONP stage after culturing in large bioreactor). Single cell survival for transplantation and maturation in vivo may be tested and the impurities of the cells can also be characterized. Cells harvested on day 25 may be seeded for spheroid formation by seeding in a PBS wheel as single cells culture for 7 days. Spheroids may be formed in Aggrewell plates for homogonous spheroids (final product). The final result of the exemplary protocol may result in a thaw and inject (TAI) formulation (single cells/spheroids).

FIG. 10 shows an exemplary batch release profile for sensory neuronal progenitor cells in accordance with the present disclosure.

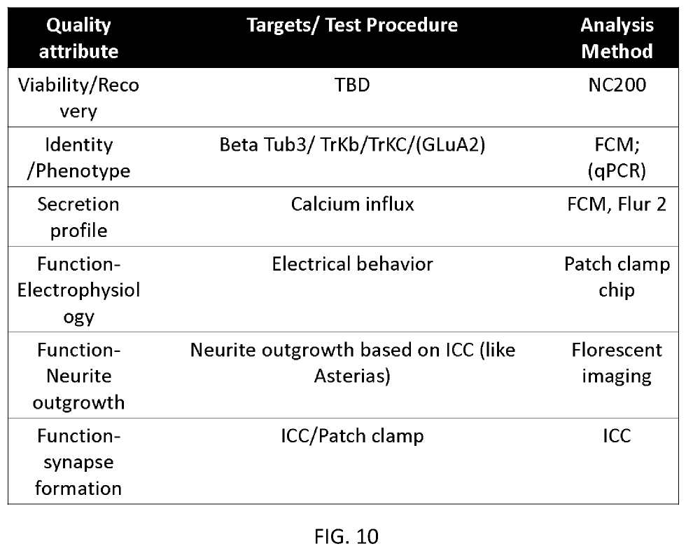

FIG. 11 shows an exemplary batch release marker profile for sensory neuronal progenitor cells in accordance with the present disclosure.

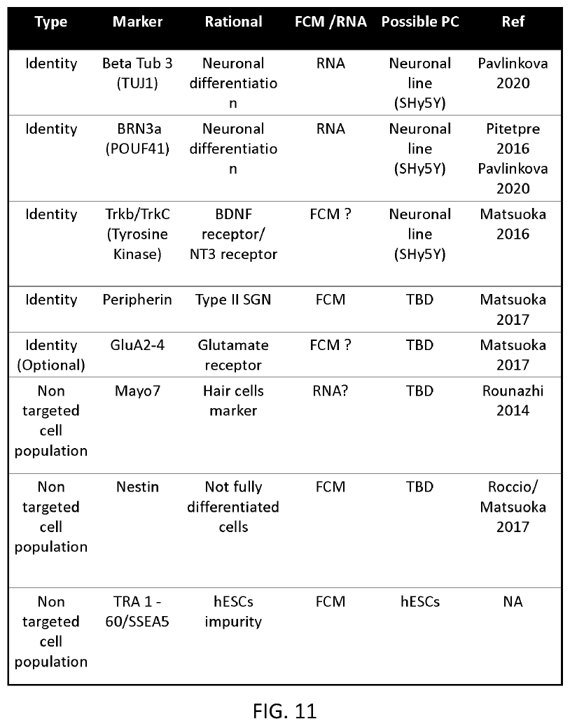

FIG. 12 shows an exemplary in-process control (IPC) test scheme for directed differentiation of human embryonic stems cells toward otic neuronal progenitor cells in accordance with the present disclosure.

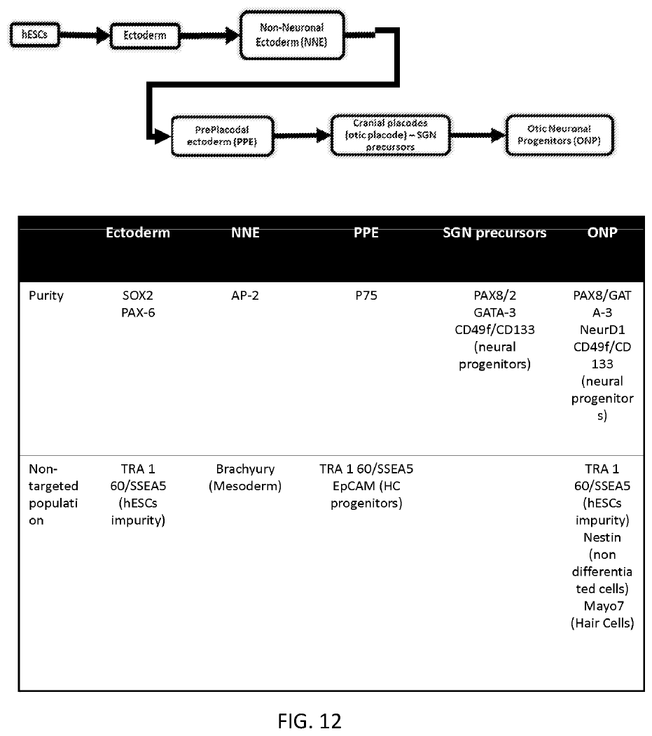

FIG. 13 shows an exemplary IPC and marker profile for sensory neuronal progenitor cells in accordance with the present disclosure.

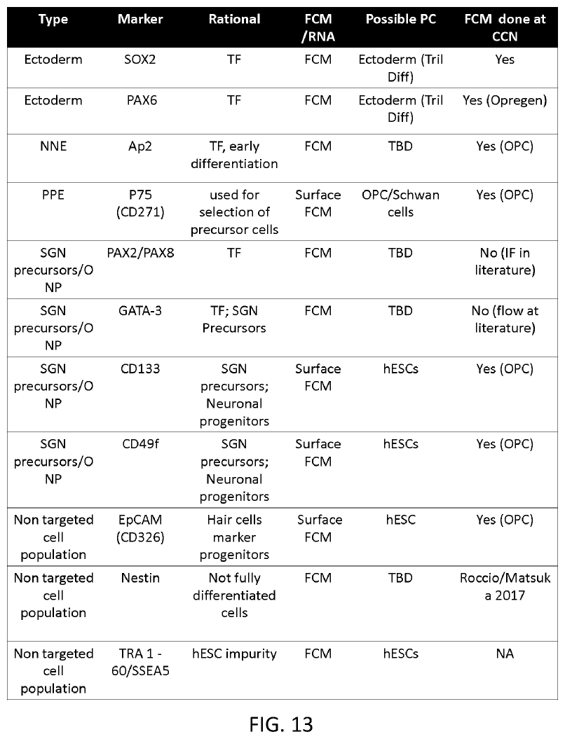

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to affect a disavowal of any part of the full scope of the invention. Hence, the following descriptions are intended to illustrate some particular aspects of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

Methods disclosed herein can comprise one or more steps or actions for achieving the described method. The method steps and/or actions may be interchanged with one another without departing from the scope of the present invention. In other words, unless a specific order of steps or actions is required for proper operation of the aspect, the order and/or use of specific steps and/or actions may be modified without departing from the scope of the present invention.

TABLE 1
Abbreviations

BDNF—Brain-derived neurotrophic factor
BMP—bone morphogenic protein (Dorsal patterning, Proliferative agent)
Brn3a—Brain specific homeobox/POU domain protein 3a
EpCAM—Epithelial cell adhesion molecule
FCM—Flow cytometry
FGF—Fibroblast growth factor (Proliferative agent, embryonic Posterior
patterning)
GATA-3—transcription factor that bind to the DNA sequence “GATA”
GF(s)—Growth Factor(s)
GluA2—Glutamate receptor A2
GluA4—Glutamate receptor A4
IF—immunofluorescence staining
IGF—Insulin growth factor
NeuroD1 neurogenic differentiation factor 1
DLX3—Distal-Less Homeobox
NNE—Non-Neuronal Ectoderm
NT3—neurotrophin 3
PAX (2,6,8)—Paired box protein, master transcription factor
PPE—Pre-Placodal ectoderm
RA—Retinoic Acid (embryonic anterior patterning, promoting towards
ectoderm)
RPE—Retinal pigmented epithelial cells
SHH—Sonic hedgehog (Drosophila mutation, embryonic ventral
patterning)
Sox2—sex determining region Y-box 2, neural stem cells pluripotency
TGF—Transforming growth factor beta (Proliferative agent)
TrkB/TrkC—Tyrosine receptor kinase B/C
TUJ1—βIII-tubulin
VGLUT1—Vesicular glutamate transporter
Wnt—(Drosophila mutations Wingless and Int-1, embryonic dorsal
patterning)
Forkhead Box G1—FOXG1
MAF BZIP Transcription Factor B—MafB

### Definitions

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations of ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

An “effective amount” is an amount sufficient for a composition to accomplish a stated purpose relative to the absence of the composition (e.g. achieve the effect for which it is administered, treat a disease, reduce enzyme activity, increase enzyme activity, reduce a signaling pathway, or reduce one or more signs or symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug (e.g., the cells described herein) is an amount of the drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). For any composition described herein, the therapeutically effective amount can be initially determined from cell culture assays. Target concentrations will be those concentrations of active composition(s) (e.g., cell concentration or number) that are capable of achieving the methods described herein, as measured using the methods described herein or known in the art.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a composition as described herein (including embodiments and examples).

As used herein, “implantation” or “transplantation” refers to the administration of a cell population into a target tissue using a suitable delivery technique, (e.g., using an injection device).

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions of the disclosure. One of skill in the art will recognize that other pharmaceutical excipients are useful in the present disclosure.

As used herein, a “patient” or “subject” refers to a living organism suffering from or prone to a disease or condition that can be treated by administration of a pharmaceutical composition, or an implantable biodegradable scaffold as provided herein. Non-limiting examples include humans, other mammals, bovines, rats, mice, dogs, monkeys, goat, sheep, cows, deer, and other non-mammalian animals. In some embodiments, a patient is human.

As used herein, a “subject in need thereof” refers to an animal or a human having damaged tissue in the central nervous system. In an embodiment, an animal or a human is experiencing a loss of motor function.

As used herein, “treatment” or “treating,” with respect to a condition or a disease, is an approach for obtaining beneficial or desired results including preferably clinical results after a condition or a disease manifests in a subject. Beneficial or desired results with respect to a disease include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival, and any combination thereof. Likewise, for purposes of this disclosure, beneficial or desired results with respect to a condition include, but are not limited to, one or more of the following: improving a condition, curing a condition, lessening severity of a condition, delaying progression of a condition, alleviating one or more symptoms associated with a condition, increasing the quality of life of one suffering from a condition, prolonging survival, and any combination thereof.

As used herein, by “inner ear sensory hair cells” or simply “hair cells” it is meant the mechanosensory hair cells of the cochlea (the auditory system) and of the saccule, utricle, crista ampularis, and semicircular canals (the vestibular system), which contribute to detecting and amplifying sound and to maintaining balance, respectively. Hair cells resemble columnar cells, each with a hair bundle of stereocilia at the apical surface. The deflection of the stereocilia opens mechanically gated ion channels that allow small, positively charged ions (primarily potassium and calcium) to enter the hair cell. Unlike many other electrically active cells, the hair cell itself does not fire an action potential. Rather, the influx of positive ions depolarizes the cell, resulting in a receptor potential. As such, hair cells typically show a graded electrical response rather than action potential spikes typical of other neurons. Hair cells may express detectable levels of one or more of the following markers: atonal homolog 1 (Atoh1/MATHI/HATHI), myosin VI (MY06), myosin VIIA (MYO7A), Espin (ESPN), myosin heavy chain 3 (MYH2), cadherin23 (CDH23), protocadherin 15 (PCDH15), otoferlin (OTOF), and prestin (SLC26A5).

As used herein, by “inner ear supporting cells”, or simply “supporting cells” it is meant the cells that contribute to the complex structural and functional properties of the cochlea, e.g., Deiters' (phalangeal) cells, Hensen's cells, Claudius cells, Boettcher cells, pillar cells, marginal cells, and the like, and of the saccule, utricle, crista ampularis, and semicircular canals. Supporting cells are identifiable by short microvilli at their apical cell surface. In addition, they are found in close proximity to hair cells, i.e. they are found directly adjacent to hair cells, as clusters with hair cells. Supporting cells may express detectable levels of one or more of the following markers: cyclin-dependent kinase inhibitor 1B (CDKNIB, p27 (KIP1)), prospero homeobox 1 (PROX1), otoancorin (OTOA), musashi homolog 1 (MSI1), SRY-box 2 (SOX2), gap junction protein beta 2, 26 kDa (Connexin 26), gap junction protein beta 6, kDa (Connexin30), gap junction protein alpha 1, 43 kDa (Connexin43), hairy/enhancer-of-split related with YRPW motif 2 (HEY2).

As used herein, “pluripotent stem cell” or “pluripotent cell” it is meant a cell that has the ability to differentiate into all types of cells in an organism. Pluripotent cells are capable of forming teratomas and of contributing to ectoderm, mesoderm, or endoderm tissues in a living organism. Examples of pluripotent stem cells are embryonic stem (ES) cells, embryonic germ stem (EG) cells, and induced pluripotent stem (iPS) cells.

As used herein, “embryonic stem cell” or “ES cell” it is meant a cell that a) can self-renew, b) can differentiate to produce all types of cells in an organism, and c) is derived from the inner cell mass of the blastula of a developing organism. ES cells can be cultured over a long period of time while maintaining the ability to differentiate into all types of cells in an organism. In culture, ES cells typically grow as flat colonies with large nucleo-cytoplasmic ratios, defined borders and prominent nuclei. In addition, ES cells express stage-specific embryonic antigen (SSEA) 5 (SSEA-5), POU class 5 homeobox 1 (Oct-4), Nanog homeobox (Nanog), SSEA-3, SSEA-4, TRA-1-60 antigen (TRA-1-60), TRA-1-81 antigen (TRA-1-81), and Alkaline Phosphatase, but not SSEA-1. Examples of methods of generating and characterizing ES cells may be found in, for example, U.S. Pat. Nos. 7,029,913, 5,843,780, and 6,200,806, the disclosures of which are incorporated herein by reference.

As used herein, by “embryonic germ stem cell”, embryonic germ cell” or “EG cell” it is meant a cell that a) can self-renew, b) can differentiate to produce all types of cells in an organism, and c) is derived from germ cells and germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98:113; Shamblott, M., et al. (1998) Proc. Natl. Acad. Sci. USA, 95:13726; and Koshimizu, U., et al. (1996) Development, 122:1235, the disclosures of which are incorporated herein by reference.

As used herein, “induced pluripotent stem cell” or “iPS cell” it is meant a cell that a) can self-renew, b) can differentiate to produce all types of cells in an organism, and c) is derived from a somatic cell. iPS cells have an ES cell-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, SRY-box transcription factor 2 (Sox2), Oct-4, Nanog, TRA-1-60, TRA-1-81, teratocarcinoma-derived growth factor 1 (TDGF1), DNA methyltransferase 3 beta (Dnmt3b), forkhead box D3 (FoxD3), growth differentiation factor 3 (GDF3), cytochrome P450 family 26 subfamily A member 1 (Cyp26a1), telomerase reverse transcriptase (TERT), and ZFP42 zinc finger protein (zfp42). iPS cells may be generated by providing the cell with “reprogramming factors”, i.e. one or more, i.e. a cocktail, of biologically active factors that act on a cell to alter transcription, thereby reprogramming a cell to pluripotency. These reprogramming factors may be provided to the cells individually or as a single composition, that is, as a premixed composition, of reprogramming factors. The factors may be provided at the same molar ratio or at different molar ratios. The factors may be provided once or multiple times in the course of culturing the cells of the subject invention. Examples of methods of generating and characterizing iPS cells may be found in, for example, Application Nos. US20090047263, US20090068742, US20090191159, US20090227032, US20090246875, and US20090304646, the disclosures of which are incorporated herein by reference.

It is appreciated that commercially available stem cells can also be used in aspects and embodiments of the present disclosure. Human ES cells may be purchased from the NIH human embryonic stem cells registry, www.grants.nih.govstem_cells/or from other hESC registries. Non-limiting examples of commercially available embryonic stem cell lines include H1, HAD-C 102, ESI, BGO 1, BG02, BG03, BG04, CY12, CY30, CY92, CYIO, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WAO 1, UCSF4, NYUES 1, NYUES2, NYUES3, NYUES4, NYUESS, NYUES6, NYUES7, UCLA 1, UCLA 2, UCLA 3, WA077 (H7), WA09 (H9), WA 13 (H13), WA14 (H14), HUES 62, HUES 63, HUES 64, CT I, CT2, CT3, CT4, MA135, Eneavour-2, WIBR 1, WIBR2, WIBR3, WIBR4, WIBRS, WIBR6, HUES 45, Shef 3, Shef 6, BINhem 19, BJNhem20, SAGO 1, and SAO01.

As used herein, “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which may be able to self-renew and naturally give rise to all or some cell types of the central nervous system but cannot give rise to cells of the mesoderm or endoderm lineages.

As used herein, “endoderm” it is meant the germ layer formed during animal embryogenesis that gives rise to the gastrointestinal tract, respiratory tract, endocrine glands and organs, certain structures of the auditory system, and certain structures of the urinary system.

As used herein, “mesoderm” it is meant the germ layer formed during animal embryogenesis that gives rise to muscles, cartilage, bones, dermis, the reproductive system, adipose tissue, connective tissues of the gut, peritoneum, certain structures of the urinary system, mesothelium, notochord, and spleen.

As used herein, “ectoderm” it is meant the germ layer formed during animal embryogenesis that gives rise to the nervous system, tooth enamel, epidermis, hair, nails, and linings of mucosal tissues. During embryogenesis, the embryonic ectoderm is patterned into lineage progenitors for neural plate, neural crest, placodes and epidermis. “Non-neuronal ectoderm” or “non-neural ectoderm” refers to ectodermal cells that will form non-neuronal structures, such as epidermis.

As used herein, “anterior ectoderm” it is meant the region of the ectodermal germ layer at the anterior, or “rostral”, end of the embryo, i.e. towards the head region. Anterior ectoderm comprises pre-placodal ectoderm and adjacent tissues such as presumptive early ectoderm, presumptive neural crest, and neural tissue. Ectoderm may be induced to become anterior ectoderm by contact with rostralizing factors such as IGF1 or insulin.

As used herein, “pre-placodal ectoderm” it is meant the narrow band of cells in the anterior ectoderm that surrounds the anterior neural plate at the end of gastrulation and that gives rise to cranial placodes, which in turn give rise to the paired sensory structures of the head. Pre-placodal ectoderm cells may express detectable levels of one or more of markers including but not limited to Neurotrophin receptor (CD271/NGFR/p75NTR), fibroblast growth factor receptor 1 (FGFR1), fibroblast growth factor receptor 2 (FGFR2), fibroblast growth factor receptor 3 (FGFR3), SIX homeobox 1 (SIX1), SIX homeobox 4 (SIX4), eyes absent homolog 1 (EYA1), and eyes absent homolog 2 (EYA2). Pre-placodal ectodermal cells are competent to respond to otic induction, that is, the induction of otic progenitor cells by culturing in the presence of FGFs, resulting in the upregulation of p75, Pax8, Pax2, GATA3 and Sox10 expression. Cells expressing pre-placodal ectodermal markers, and which have pre-placodal ectodermal characteristics, can be induced from undifferentiated pluriplotent stem cells using the methods described herein.

As used herein, by “otic progenitor cells” or “otic neural progenitor cells” it is meant a somatic cell that a) can self-renew, and b) can differentiate to give rise to inner ear sensory hair cells, auditory neurons, and supporting cells. Otic progenitor cells grow as spheres of cells when cultured in non-adherent conditions, or as clusters of cells when cultured in adherent conditions. Furthermore, otic progenitor cells may express detectable levels of one or more of the following markers: paired box 2 (PAX2), paired box 8 (PAX8), distal-less homeobox 5 (DLX5), orthodenticle homeobox 2 (OTX2), eyes absent homolog 1 (EYA1), SIX homeobox 1 (SIX1), jagged 1 (JAG1), fibroblast growth factor receptor 1 (FGFR1). Other markers include forkhead box 13 (FOXI3), SRY-box 2 (SOX2), NOTCHI, delta-like 1 (DELTA1), bone morphogenetic protein 7 (BMP7), T-box 1 (TBX1), GATA binding protein 3 (GATA3), forkhead box D3 (FOXD3), hairy/enhancer-of-split related with YRPW motif 1 (HEY1), hairy/enhancer-of-split related with YRPW motif 2 (HEY2), hairy and enhancer of split 1 (HES1), hairy and enhancer of split 6 (HES6), Activin receptor (ACTIVIN-R), H6 family homeobox 3 (NKX5.1), Claudin 8 (CLDN8), Claudin 14 (CLDN14). Otic neural progenitor cells can be divided into early, mid, and late otic progenitor cells based on marker expression, as described herein.

As used herein, “stromal cells” it is meant connective tissue cells of any organ, e.g. fibroblasts, pericytes, endothelial cells, etc.

As used herein, the term “microcarrier” or “MC” refers to a suspendible support matrix that allows adherent cells to grow in dynamic or static cell culture, and can stay in suspension with gentle mixing. Microcarriers can be composed of including, but not limited to, polystyrene, surface-modified polystyrene, chemically modified polystyrene, cross-linked dextran, cellulose, acrylamide, collagen, alginate, gelatin, glass, DEAE-dextran, or a combination thereof. Microcarriers can be coated with a biological support matrix, including, but not limited to, laminin, Matrigel®, collagen, poly-lysine, poly-L-lysine, poly-D-lysine, vitronectin, fibronectin, tenascin, dextran, a peptide, or a combination thereof. Many different types of microcarriers are commercially available, including, but not limited to, HyQSphere (HyClone™), Hillex (SoloHill Engineering), and Low Concentration Synthemax® II (Corning) brands. Microcarriers can be made from cross-linked dextran such as the Cytodex® brand (GE Healthcare). Microcarriers can be spherical and smooth, can have microporous surfaces, such as CYTOPORE™ brand (GE Healthcare), and/or can be rod-shaped carriers such as DE-53 (Whatman™). Microcarriers can be impregnated with magnetic particles that may help in cell separation from beads (e.g., GEM particles from Global Cell Solutions). Chip-based microcarriers such as the uHex product (Nunc) provide a flat surface for cell growth while maintaining the high surface to volume ratio of traditional microcarriers. The properties of microcarriers may significantly affect expansion rates and cell multi- or pluripotency.

As used herein, “dynamic culture” it is meant cell cultivation that, unlike cell cultivation performed in static conditions (e.g., petri dishes), is conducted with intentional active motion to enhance mass transfer and mechanotransductive effects (e.g., bioreactors) which often results in higher numbers of functional cells. For example, in dynamic differentiation processes, bioreactors directly apply mechanical forces to generate physiologic conditions and enhance differentiation towards a specific cell lineage. In dynamic culture, cells may also have a more homogenous environment, that diffusion alone cannot provide in static culture. For example, cells that are grown in the vessel periphery vs. vessel inner areas. Further, static culture may generate various biologically separate niches, as it sustains microenvironments with various cell densities, that are not sustainable in Dynamic culture.

As used herein, “dynamic two-dimensional” or “dynamic 2D it is meant cell that are grown and form a monolayer on microcarriers. For example, cells cultured in dynamic culture (e.g. bioreactor) with suspended adhesive agents (e.g., microcarriers) that allows attachment of the cells to form dynamic 2D culture.

As used herein, “dynamic three-dimensional” or “dynamic 3D it is meant cells that are grown as aggregates in suspension, such as a cell culture in an artificially created environment in which biological cells are permitted to grow or interact with their surroundings in all three dimensions. Unlike 2D environments, a 3D cell culture allows cells in vitro to grow in all directions, similar to how they would in vivo. These three-dimensional cultures can be, for example, grown in bioreactors, small capsules in which the cells can grow into spheroids, or 3D cell aggregates.

As used herein, by “sensory neuronal progenitor cells” it is meant are self-renewing, multipotent cells that firstly generate the radial glial progenitor cells that generate the neurons and glia of the nervous system of all animals during embryonic development. While sensory neuronal progenitor cells can be naturally occurring, their cellular composition differs from the cells induced from undifferentiated pluripotent stem cells using the methods disclosed herein.

As used herein, “auditory neuron,” “auditory neurons” (abbreviated AN), “auditory cell” or “auditory cells” refers, or refer, to sensory cell populations of the ear including, but not intended to be limited to, one or more of hair cells, supporting cells, otic progenitor cells, sensory neuronal progenitor cells, and the like. The term “auditory cells” may, in some cases, refer to a mixed population of cells encompassing any combination of the cell types described above, in any ratio.

As used herein, ‘auditory disorder’ or ‘auditory condition’ or ‘hearing disorder’ or ‘hearing condition’ refers to conditions or disorders including but not intended to be limited to conductive hearing loss, sensorineural hearing loss, central hearing loss, mixed hearing loss, auditory neuropathy spectrum disorder, central auditory processing disorder and tinnitus.

As used herein, ‘conductive hearing loss’ refers to the impaired transmission of sound waves through the external ear canal to the bones of the middle ear.

As used herein, ‘sensorineural hearing loss’ refers to a pathologic change in structures with the inner ear or in the acoustic nerve.

As used herein, ‘central hearing loss’ refers to a pathologic condition above the junction of the acoustic nerve and the brainstem.

As used herein, ‘mixed hearing loss’ refers to a subject having both conductive hearing loss and sensorineural hearing loss.

As used herein, ‘auditory neuropathy spectrum disorder’ refers to a type of sensorineural hearing loss where the auditory nerve fails to send consistent messages to the auditory centers of the brain.

As used herein ‘central auditory processing disorder’ refers to deficits in the neural processing of auditory information in the central auditory nervous system.

### Methods of Producing Populations of Auditory Cells

The disclosure provides methods of producing populations of auditory cells from undifferentiated pluripotent stem cells. The methods comprise culturing populations of undifferentiated pluripotent stem cells in different combinations of growth factors and growth factor inhibitors, in a series of steps that induces the differentiation of the undifferentiated pluripotent stems cells towards auditory neuronal fates through a series of differentiation steps. In an exemplary differentiation pathway, human embryonic stem cells (hESCs) are induced to differentiate into non-neuronal ectoderm (NNE) cells, which are induced to differentiate into pre-placodal ectoderm (PPE) cells, which in turn are induced to different into early otic neuronal progenitor (ONP) cells, mid otic progenitor cells, late otic progenitor cells, and spiral ganglion neurons. The resulting population of cells may contain a mixture of cell types. However, cells from the later stages of the pathway may predominate, and residual hESCs may be minimal or absent. Without wishing to be bound by theory, it is thought that a composition comprising a mixed cell population may by better suited as a therapeutic agent for auditory diseases and disorders than a composition comprising a homogenous population of cells, as the range of cell types increases the niches into which the cells can engraft when administered to a subject, and the increases the number of fates that the cells can adopt upon administration.

Methods for Expanding and Maintaining Human Embryonic Stem Cells (hESCs)

In an aspect, provided herein are methods for expanding and maintaining human embryonic stem cells (hESCs) in an undifferentiated, pluripotent state, the method comprising the steps of (a) simultaneously combining human embryonic stem cells and an extracellular matrix component (ECM) in growth media in tissue culture flasks for static expansion, and (b) culturing the adherent hESCs for a period of time.

In some embodiments, the cultured human embryonic stem cells of the static expansion are harvested non-enzymatically using ReLeSR™ and cultured in mTeSR™ plus media on iMatrix-511 coated vessels. In some embodiments, hESCs are expanded further by repeating steps (a) and (b).

In some embodiments, the cultured human embryonic stem cells of the static expansion are harvested and further differentiated.

In an aspect, provided herein are methods for expanding and maintaining human embryonic stem cells (hESCs) in an undifferentiated, pluripotent state, the method comprising the steps of (a) simultaneously combining human embryonic stem cells, an extracellular matrix component (ECM), and a microcarrier in growth media to form a suspendable expansion complex, and (b) culturing the suspendable expansion complex for a period of time.

In some embodiments, the cultured human embryonic stem cells of the suspendable expansion complex are harvested and expanded further by repeating steps (a) and (b).

In some embodiments, the cultured human embryonic stem cells of the suspendable expansion complex are harvested and further differentiated.

Human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by a procedure in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells, see Reubinoff et al. Nat Biotechnol 2000 May: 18 (5): 559; Thomson et al., [U.S. Pat. No. 5,843,780; Science 282:1145, 1998; Curr. Top. Dev. Biol. 38:133, 1998; Proc. Natl. Acad. Sci. USA 92:7844, 1995]; Bongso et al., [Hum Reprod 4:706, 1989]; and Gardner et al., [Fertil. Steril. 69:84, 1998].

In addition, ES cells can be obtained from other species, including mouse (Mills and Bradley, 2001), golden hamster [Doetschman et al., 1988, Dev Biol. 127:224-7], rat [Iannaccone et al., 1994, Dev Biol. 163:288-92], rabbit [Giles et al. 1993, Mol Reprod Dev. 36:130-8; Graves & Moreadith, 1993, Mol Reprod Dev. 1993, 30 36:424-33], several domestic animal species [Notarianni et al., 1991, J Reprod Fertil Suppl. 43:255-60; Wheeler 1994, Reprod Fertil Dev. 6:563-8; Mitalipova et al., 2001, Cloning. 3:59-67] and non-human primate species (Rhesus monkey and marmoset) [Thomson et al., 1995, Proc Natl Acad Sci USA. 92:7844-8; Thomson et al., 1996, Biol Reprod. 55:254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, MO, USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG (embryonic germ) cells are prepared from the primordial germ cells obtained from fetuses of about 8-11 weeks of gestation (in the case of a human fetus) using laboratory techniques known to anyone skilled in the arts. The genital ridges are dissociated and cut into small portions which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95:13726, 1998] and U.S. Pat. No. 6,090,622.

Yet another method for preparing ES cells is by parthenogenesis. The embryo is also not destroyed in the process.

The cells may be expanded in suspension, with or without a microcarrier, or in a monolayer. The expansion of the mixed population of cells in monolayer cultures or in suspension culture may be modified to large scale expansion in bioreactors or multi/hyper stacks by methods well known to those versed in the art.

According to some embodiments, the expansion phase is effected for at least one to 20 weeks, for example at least one week, at least 2 weeks, at least 3 weeks, at least 4 weeks, at least 5 weeks, at least 6 weeks, at least 7 weeks, at least 8 weeks, at least 9 weeks or even 10 weeks. In embodiments, the expansion phase is effected for 1 week to 10 weeks, such 2 weeks to 10 weeks, 3 weeks to 10 weeks, 4 weeks to 10 weeks, or 4 weeks to 8 weeks. The time period may be any value or subrange within the recited ranges, including endpoints.

According to still other embodiments, the expansion phase is effected until a suitable lactate concentration in the cell culture medium, and/or precent confluence is achieved. Percent confluence is the percentage of the culture vessel surface area that appears covered by a layer of cells when observed by microscopy. In some embodiments, the undifferentiated pluripotent stem cells are cultured until the lactate concentration in the cell culture medium is between about 1.0 to 13. 0 mM, or between about 1.5-12.5 mM. In some embodiments, cells are cultured until the lactate concentration in the cell culture medium is between about 1.68-12.29 mM. In some embodiments, the percent confluence is between 5% and 85%.

According to still other embodiments, the mixed population of cells are passaged at least one time during the expansion phase, at least twice during the expansion phase, at least three times during the expansion phase, at least four times during the expansion phase, at least five times during the expansion phase, at least six times during the expansion phase, or at least seven times during the expansion phase.

When cells are collected enzymatically, it is possible to continue the expansion for more than 8 passages, more than 9 passages and even more than 10 passages (e.g. 11-15 passages). The number of total cell doublings can be increased to greater than 30, e.g. 31, 32, 33, 34 or more. (See international patent application publication number WO 2017/021973, incorporated herein by reference in its entirety).

An extracellular matrix (ECM) is a three-dimensional network consisting of extracellular macromolecules and minerals, such as collagen, enzymes, glycoproteins and hydroxyapatite that provide structural and biochemical support to surrounding cells. Because multicellularity evolved independently in different multicellular lineages, the composition of ECM varies between multicellular structures; however, cell adhesion, cell-to-cell communication and differentiation are common functions of the ECM.

The animal extracellular matrix includes the interstitial matrix and the basement membrane. Interstitial matrix is present between various animal cells (i.e., in the intercellular spaces). Gels of polysaccharides and fibrous proteins fill the interstitial space and act as a compression buffer against the stress placed on the ECM. Basement membranes are sheet-like depositions of ECM on which various epithelial cells rest. Each type of connective tissue in animals has a type of ECM: collagen fibers and bone mineral comprise the ECM of bone tissue; reticular fibers and ground substance comprise the ECM of loose connective tissue; and blood plasma is the ECM of blood.

Suitable extracellular matrix components for use within the scope of the present disclosure may include, but are not necessarily limited to, Matrigel®, vitronectin, gelatin, collagen I, collagen IV, laminin (e.g. laminin 521), fibronectin poly-D-lysine, their derivatives, or a combination thereof. In specific embodiments, the human laminin is human laminin 511 E8 fragment.

In some embodiments, the microcarriers may comprise one or more of polystyrene, cross-linked dextran, magnetic particles, microchips, cellulose, hydroxylated methacrylate, collagen, gelatin, polystyrene, plastic, glass, ceramic, or silicone. In some embodiments, the microcarriers are composed of polystyrene, surface-modified polystyrene, chemically modified polystyrene, cross-linked dextran, cellulose, acrylamide, collagen, alginate, gelatin, glass, DEAE-dextran, or a combination thereof. In some embodiments, the microcarrier is composed of polystyrene. In some embodiments, the microcarrier is composed of surface-modified polystyrene. In some embodiments, the microcarrier is composed of chemically modified polystyrene. In some embodiments, the microcarrier is composed of cross-linked dextran. In some embodiments, the microcarrier is composed of cellulose. In some embodiments, the microcarrier is composed of acrylamide. In some embodiments, the microcarrier is composed of collagen. In some embodiments, the microcarrier is composed of alginate. In some embodiments, the microcarrier is composed of gelatin. In some embodiments, the microcarrier is composed of glass. In some embodiments, the microcarrier is composed of DEAE-dextran. In some embodiments, the microcarriers are not coated.

In some embodiments, the microcarriers are coated. In embodiments, the microcarriers may be coated with Matrigel®, laminin, vitronectin, collagen, their derivatives, or a combination thereof. In embodiments, the microcarriers may be coated by poly-lysine, poly-L-lysine, poly-D-lysine, fibronectin, tenascin, dextran, a peptide, or a combination thereof. In some embodiments, the microcarrier is coated with laminin. In some embodiments, the microcarrier is coated with Matrigel®. In some embodiments, the microcarrier is coated with collagen. In some embodiments, the s microcarrier is coated with poly-lysine. In some embodiments, the microcarrier is coated with poly-L-lysine. In some embodiments, the microcarrier is coated with poly-D-lysine. In some embodiments, the microcarrier is coated with vitronectin. In some embodiments, the microcarrier is coated with fibronectin. In some embodiments, the microcarrier is coated with tenascin. In some embodiments, the microcarrier is coated with dextran. In some embodiments, the microcarrier is coated with a peptide.

In some embodiments, the microcarriers may be spherical, smooth, macroporous, rod-shaped, or a combination thereof. In some embodiments, the microcarriers may be coupled with protamine or polylysine. In some embodiments, the microcarrier is spherical. In some embodiments, the s microcarrier is ellipsoidal. In some embodiments, the microcarrier is rod-shaped. In some embodiments, the microcarrier is disc-shaped. In some embodiments, the microcarrier is porous. In some embodiments, the microcarrier is non-porous. In some embodiments, the microcarrier is smooth. In some embodiments, the microcarrier is flat.

In some embodiments, the microcarriers are neutral. In some embodiments, the microcarriers are negatively charged. In some embodiments, the microcarriers are hydrophilic.

In some embodiments, the microcarriers may have a surface area of 25 cm2, 50 cm2, 75 cm2, 100 cm2, 125 cm2, 150 cm2, 175 cm2, 200 cm2, 225 cm2, 250 cm2, 500 cm2, 625 cm2, 750 cm2, 1,000 cm2, 1,250 cm2, 5,000 cm2, or 7,500 cm2. The surface area may be any value or subrange within the recited ranges, including endpoints.

In specific embodiments, the microcarriers are surface treated to enhance cell attachment, maximizing cell yield and viability. The microcarriers may be comprised of USP Class VI polystyrene material, which provides a consistent platform. In some embodiments, the microcarriers create a synthetic surface on the microcarriers for stem cell expansion. An enhanced attachment surface treatment infuses the surface of the microcarriers with oxygen to improve cell attachment. In some embodiments, the microcarriers are nonpyrogenic. In some embodiments, the microcarriers are optimized for mesenchymal stem cell applications. In specific embodiments, the beads may vary in size from 125-212 μm. In specific embodiments, the density of the microcarriers may be 1.026±0.004. In specific embodiments, the microcarriers may be 360 cm2/gram.

In some embodiments, the method comprises combining the hESCs with laminin or a derivative thereof to improve the cell attachment to the carrier surface. In specific embodiments, the laminin is human laminin 511. As alternative embodiments, several other extracellular matrices may be used for cell attachment, such as including, but not necessarily limited to, vitronectin, fibronectin, collagen, Matrigel®, or derivatives thereof.

In some embodiments, the cells may be cultured for one day, two days, three days, four days, five days, six days, seven days, eight days, nine days, ten days, eleven days, twelve days, thirteen days, or fourteen days.

In some embodiments, the cells may be cultured in a working volume of between 10 mL and 3,000 mL, for example about 10 mL, 20 mL, 30 mL, 40 mL, 50 mL, 100 mL, 250 mL, 500 mL, 750 mL, 1,000 mL, or 3,000 mL. The volume may be any value or subrange within the recited ranges, including endpoints.

In some embodiments, the cultured cells may be expanded further.

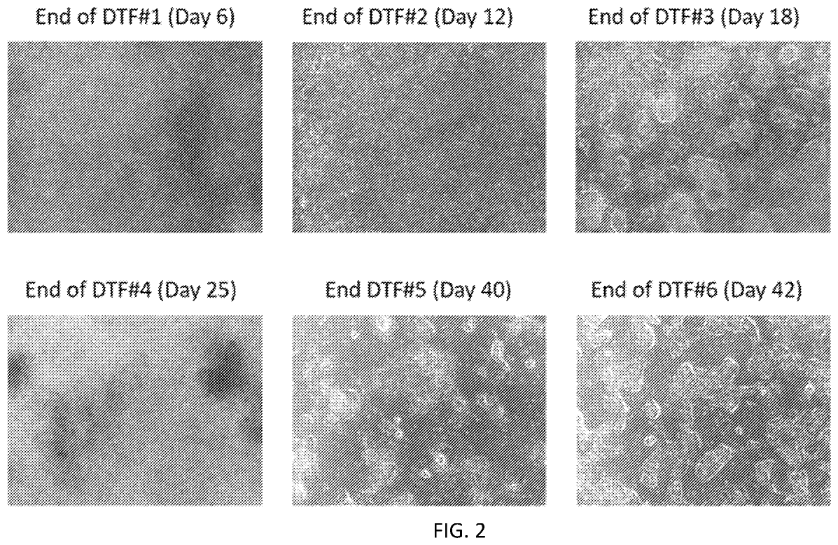

In some embodiments, the cultured cells may remain undifferentiated. Undifferentiated cells may be identified by expression of various markers, such as including, but not necessarily limited to, SSEA-5, TRA-1-60, Oct-4, and Nanog. In some embodiments, undifferentiated cells express SSEA-5. In some embodiments, undifferentiated cells express TRA-1-60. In some embodiments, undifferentiated cells express Oct-4. In some embodiments, undifferentiated cells express Nanog. In some embodiments, undifferentiated cells express both SSEA-5 and TRA-1-60. In some embodiments, undifferentiated cells express both Oct-4 and Nanog. In some embodiments, undifferentiated cells express SSEA-5, TRA-1-60, Oct-4, and Nanog (See FIG. 2 IPC #0).

In some embodiments, the cells may be cultured in a feeder cell-conditioned medium. ES culturing methods may include the use of feeder cell layers which secrete factors needed for stem cell proliferation, while at the same time, inhibiting their differentiation. The culturing is typically effected on a solid surface, for example a surface coated with gelatin or vimentin. Exemplary feeder layers include human embryonic fibroblasts, adult fallopian epithelial cells, primary mouse embryonic fibroblasts (PMEF), mouse embryonic fibroblasts (MEF), murine fetal fibroblasts (MFF), human embryonic fibroblast (HEF), human fibroblasts obtained from the differentiation of human embryonic stem cells, human fetal muscle cells (HFM), human fetal skin cells (HFS), human adult skin cells, human foreskin fibroblasts (HFF), human umbilical cord fibroblasts, human cells obtained from the umbilical cord or placenta, and human marrow stromal cells (hMSCs). Growth factors may be added to the medium to maintain the ESCs in an undifferentiated state. Such growth factors include bFGF and/or TGF. In another embodiment, agents may be added to the medium to maintain the hESCs in a naive undifferentiated state-see for example Kalkan et al., 2014, Phil. Trans. R. Soc. B, 369:20130540.

hESCs are typically plated on top of the feeder cells 1˜4 days later in a supportive medium (e.g. NUTRISTEM®, NUT (+) with human serum albumin, mTeSR™ plus, or mTeSR™1 StemFit®). Additional factors may be added to the medium to prevent differentiation of the ESCs such as bFGF and TGFB3. Once a sufficient amount of hESCs is obtained, the cells may be mechanically disrupted (e.g. by using a sterile tip or a disposable sterile stem cell tool; 14602 Swemed). Alternatively, the cells may be removed by enzymatic treatment (e.g. collagenase A, or TrypLE™ Select). This process may be repeated several times to reach the necessary amount of hESC. According to some embodiments, following the first round of expansion, the hESCs are removed using TrypLE™ Select and following the second round of expansion, the hESCs are removed using collagenase A.

Feeder cell free systems have also been used in ES cell culturing, such systems utilize matrices supplemented with serum replacement, cytokines and growth factors (including IL6 and soluble IL6 receptor chimera) as a replacement for the feeder cell layer. Stem cells can be grown on a solid surface such as an extracellular matrix (e.g., MATRIGEL®, laminin or vitronectin) in the presence of a culture medium—for example the Lonza L7™ system, mTeSR™, StemPro™ XFKSR, E8, NUTRISTEM®). Unlike feeder-based cultures which require the simultaneous growth of feeder cells and stem cells and which may result in mixed cell populations, stem cells grown on feeder-free systems are easily separated from the surface. The culture medium used for growing the stem cells contains factors that effectively inhibit differentiation and promote their growth such as MEF-conditioned medium and bFGF.

Also within the scope of the present disclosure are methods for expanding and maintaining human embryonic stem cells (hESCs) in an undifferentiated state, comprising culturing human pluripotent stem cells on a non-adherent surface to obtain a population of undifferentiated hESCs, combining said population of undifferentiated hESCs with microcarriers in growth media, and expanding said population of cells.

Examples of non-adherent cell culture plates include those manufactured by Nunc (e.g. Hydrocell Cat No. 174912), etc. In other embodiments, non-adherent suspension culture dishes may be used (e.g., Corning).

According to some embodiments, when the cells are cultured on the non-adherent substrate, e.g. cell culture plates, the atmospheric oxygen conditions are 20%. However, manipulation of the atmospheric oxygen conditions is also contemplated such that the atmospheric oxygen percent is less than about 20%, 15%, 10%, 9%, 8%, 7%, 6% or even less than about 5% (e.g. between 1%-20%, 1%-10% or 0-5%). According to other embodiments, the cells are cultured on the non-adherent substrate initially under normal atmospheric oxygen conditions and then lowered to less than normal atmospheric oxygen conditions.

While methods described above are directed to methods of expanding and maintaining hESCs, analogous methods directed to induced pluripotent stem cells (iPSCs) are also within the scope of the present disclosure. iPSCs are a type of stem cell derived from somatic cells which have been reprogrammed back into a pluripotent state through the introduction of pluripotency associated genes, and are available from a variety of sources. The person of ordinary skill in the art will appreciate the changes necessary to adapt the hESC methods described above for use with iPSCs and the like.

### Expansion Compositions

In another aspect, provided herein are suspendable expansion complex compositions comprising human embryonic stem cells or IPSCs, an extracellular matrix component (ECM), and a microcarrier.

Human embryonic stem cells, extracellular matrices, and microcarriers are described in detail elsewhere herein.

Expansion complex ranges may vary. In the following tables, the range of the complex components are detailed in different units for the ECM component.

TABLE 2
Expansion complex of MC ECM and cells (ECM in μg)

| | | ECM - Laminin 511 E8 | hESCs (cells per |
|---|---|---|---|
| | MCs | fragment (μg per cm²) | cm²) |
| Min | 1 cm² | 0.125 | 9,600 |
| Max | 1 cm² | 0.125 | 50,000 |

The ECM component can be presented by mol/cm² by using the laminin 511 E8 fragment's molecular weight (150 KDa).

TABLE 3
Expansion complex of MC ECM and cells (ECM in mol)

| | | ECM - Laminin 511 E8 | hESCs (cells per |
|---|---|---|---|
| | MCs | fragment (mol per cm²) | cm²) |
| Min | 1 cm² | 8.33 × 10⁻¹³ | 9,600 |
| Max | 1 cm² | 8.33 × 10⁻¹³ | 50,000 |

The ECM component can also be presented by the number of molecules/cm² by using molecular weight (150 KDa) multiplied by Avogadro's number (6.022×10²³).

TABLE 4
Expansion complex of MC ECM media and cells (ECM molecules)

| | | ECM - Laminin 511 E8 | hESCs (cells per |
|---|---|---|---|
| | MCs | fragment (molecules per cm²) | cm²) |
| Min | 1 cm² | 5.01833 × 10¹¹ | 9,600 |
| Max | 1 cm² | 5.01833 × 10¹¹ | 50,000 |

In some embodiments, the following specification parameters may be expanded: # Of hESCs (cells)—4,000-600,000 cells per cm² of microcarriers; Laminin 511 E8 fragment (μg per cm² of microcarriers)—0.125 μg per cm² or higher.

In some embodiments, the composition may further comprise a growth medium. Non-limiting examples of commercially available basic media (i.e. a chemically defined medium or CDM) that may be utilized in accordance with this disclosure comprise NUTRISTEM® (without bFGF and TGF for ESC differentiation, with bFGF and TGF for ESC expansion), NEUROBASAL™, KO-DMEM, DMEM, DMEM/F12, CELLGRO™ Stem Cell Growth Medium, or X-VIVO™. The basic medium may be supplemented with a variety of agents as known in the art dealing with cell cultures. The following is a non-limiting reference to various supplements that may be included in the culture to be used in accordance with the present disclosure: serum or with a serum replacement containing medium, such as, without being limited thereto, knock out serum replacement (KOSR), NUTRIDOMA-CS, TCH™, N2, N2 derivative, or B27 or a combination; an extracellular matrix (ECM) component, such as, without being limited thereto, fibronectin, laminin, collagen and gelatin. In some embodiments, the cell culture medium comprises a chemically defined medium (CDM) supplemented with N2, B27, or a combination thereof, optionally supplemented (with BrainPhys™, The ECM may then be used to carry the one or more members of the TGFI3 superfamily of growth factors; an antibacterial agent, such as, without being limited thereto, L-glutamine, beta mercaptoethanol, penicillin and streptomycin; and non-essential amino acids (NEAA), neurotrophins which are known to play a role in promoting the survival of SCs in culture, such as, without being limited thereto, BDNF, NT3, NT4.

As described above, the microcarriers may comprise one or more of polystyrene, cross-linked dextran, magnetic particles, microchips, cellulose, hydroxylated methacrylate, collagen, gelatin, polystyrene, plastic, glass, ceramic, silicone. In some embodiments, the microcarrier is composed of polystyrene. In some embodiments, the microcarrier is composed of surface-modified polystyrene. In some embodiments, the microcarrier is composed of chemically modified polystyrene. In some embodiments, the microcarrier is composed of cross-linked dextran. In some embodiments, the microcarrier is composed of cellulose. In some embodiments, the microcarrier is composed of acrylamide. In some embodiments, the microcarrier is composed of collagen. In some embodiments, the microcarrier is composed of alginate. In some embodiments, the microcarrier is composed of gelatin. In some embodiments, the microcarrier is composed of glass. In some embodiments, the microcarrier is composed of DEAE-dextran.

As described above, the microcarriers may be spherical, smooth, macroporous, rod-shaped, or a combination thereof.

In some embodiments, the microcarriers may be coated with matrigel, laminin, vitronectin, collagen, their derivatives, or a combination thereof. In some embodiments, the laminin is human laminin 511.

In some embodiments, the microcarriers are not coated.

In some embodiments, the microcarriers have a surface area (per gram): of 25 cm² to 7,500 cm², e.g., about 25 cm², 50 cm², 75 cm², 100 cm², 125 cm², 150 cm², 175 cm², 200 cm², 225 cm², 250 cm², 500 cm², 625 cm², 750 cm², 1,000 cm², 1,250 cm², 5,000 cm², or 7,500 cm². The surface area may be any value or subrange within the recited ranges, including endpoints.

In some embodiments, the microcarriers are coupled with protamine or polylysine. In some embodiments, the microcarriers are neutral. In some embodiments, the microcarriers are negatively charged. In some embodiments, the microcarriers are hydrophilic.

### Methods of Making Auditory Cells

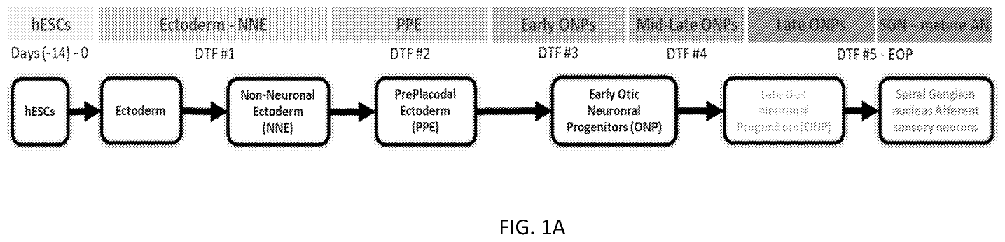

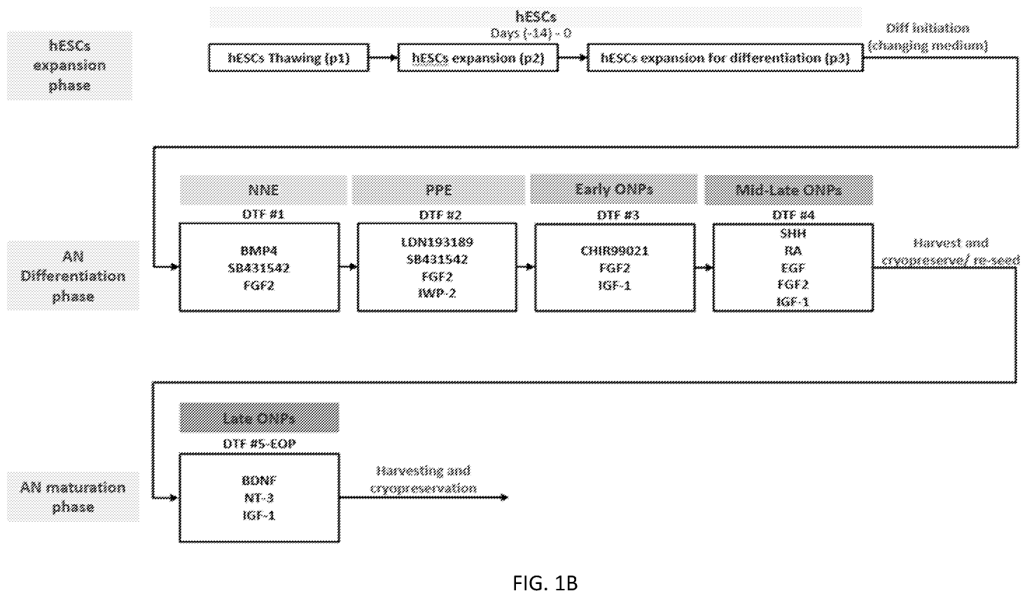

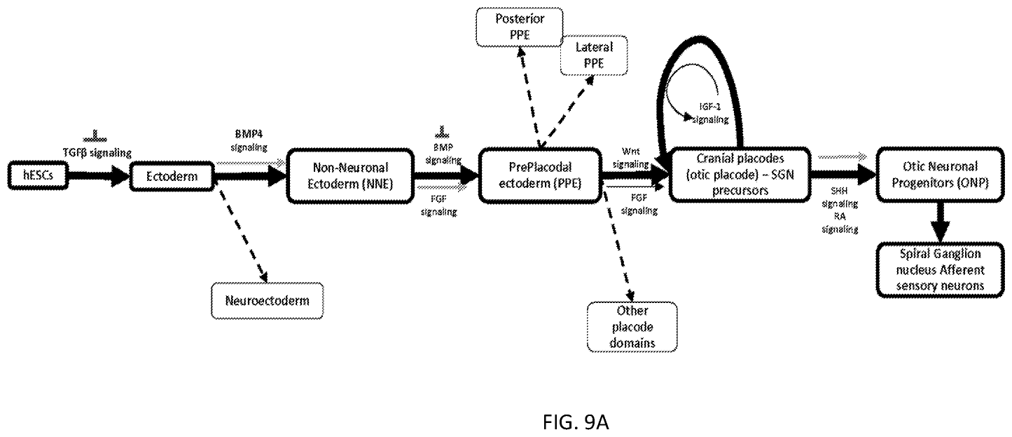

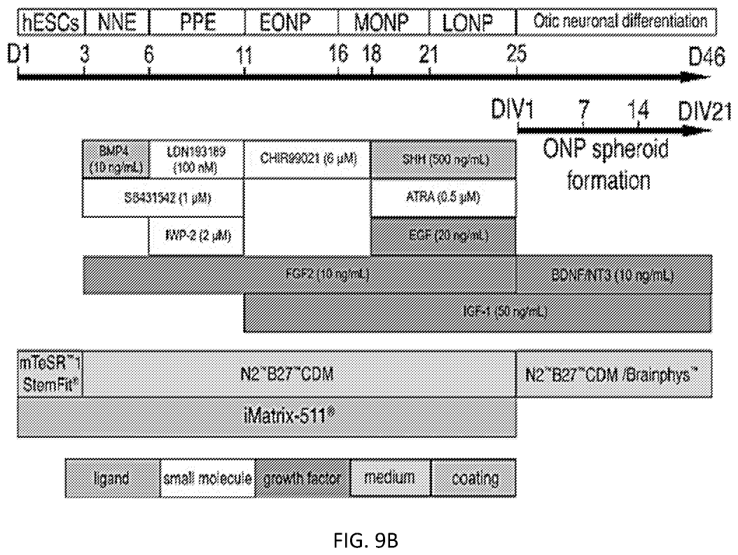

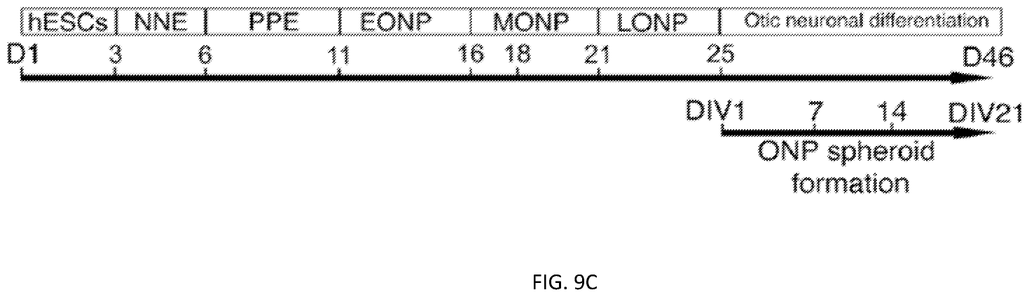

In accordance with the present disclosure, human pluripotent stem cells (hPSCs) can be grown in dynamic culture on microcarriers in a hESC culture media, and maintained in pluripotency state by daily replacement of the hPSC media, as described above. The hPSCs will be differentiated by medium replacement into a culture media (1:1 mixture of DMEM/F12 and Neurobasal medium) that will induce non neuronal ectoderm (NNE) formation. This media can include, for example, B27 and N2 supplements, TGF beta agonist such as BMP4 (1-25 ng/ml), vitamin b3 derivative nicotine amide (NIC, 1-25 mM), SB431542, and/or FGF2 (1-25 ng/ml). Dynamic of static culture will continue for 3-7 days, and medium will be replaced either fully or gradual (75-100% of volume each day). At differentiation day 4-8 differentiation factors will be replaced with FGF2, LDN193189 (20-400 nM), IWP-2 (2 μM), SB431542 (1 μM), NIC (1-25 mM), the Wnt inhibitor IWR-endo (1-10 μM), to generate the pre-placodal ectoderm Dynamic culture will continue for 3-7 days with medium replacement every 1-3 days. At differentiation day 8-14 differentiation factors will be replaced with FGF2, CHIR99021 (6 μM), and IGF1 (50 ng/ml), to generate the early ONP. Dynamic culture will continue for 7-10 days with medium replacement every 2-3 days. On day 17-22 differentiation factors will be replaced with FGF2, EGF, retinoic acid (RA 0.2-2 μM), SHH (500 ng/ml) and IGF1 (50 ng/ml), to generate the mid-Late ONP. Dynamic culture will continue for 7-10 days with medium replacement every 1-3 days. On day 22-28, Mid-Late ONP cells will be harvested and inoculated into static/dynamic suspension as single cells for 3 days in the presence of BDNF (10 ng/ml), NT3 (10 ng/mL), and IGF-1 Rock inhibitor (e.g., Y-27632 Dihydrochloride, 10 μM) to form small aggregates. At differentiation day 22-28 the cells will be further expanded for final maturation (See FIGS. 1B and 9B).

In some embodiments, the hPSCs can be, for example, differentiated by medium replacement into a culture media (1:1 mixture of DMEM/F12 and Neurobasal medium) that will induce Neural Crest formation, by culturing in 2D with 50-2000 ng/ml Noggin and 0.5-20 ng/ml FGF2 for about 14 days. On or about day 14, cells are transferred to culturing in 3D with, for example, 5-100 ng/ml EGF and 5-100 ng/ml FGF2 for about 5 days. On or about day 19, cells are returned to a 2D culturing with differentiation factors FGF2, Purmorphamine (0.1-1 μM), EGF, retinoic acid (RA 0.2-2 μM) and IGF1 (50 ng/ml), to generate the Late ONP. Dynamic culture will continue for about 7 days with medium replacement about every 2-3 days. On or about day 25, Late ONP cells will be harvested and inoculated into dynamic suspension as single cells for about 3 days in the presence of FGF and EGF and Rock inhibitor (2-50 μM) to form small aggregates.

hPSCs can be differentiated into different populations of cells through culture in a variety of different mediums comprising growth factors and growth factor inhibitors. In some embodiments, undifferentiated pluripotent stem cells are subjected to conditions sufficient for directed differentiation to produce a composition comprising a population of auditory cells, for example a population of cells comprising NNE, PPE, ONP cells, neurons (e.g., spiral ganglion neurons) or any combination thereof. In some embodiments, the method comprises culturing a population of hPSCs in 1, 2, 3, 4, or 5 culture media, each comprising a combination of growth factors and/or growth factor inhibitors, under conditions sufficient to drive the population of cells towards a target cell type, thereby producing a population of cells comprising the target cell type.

In some embodiments the method begins with seeding a population of undifferentiated pluripotent stem cells at a density of 1,200-20,000 live cells/cm², optionally in a monolayer, and culturing the cells until a lactate concentration in the cell culture medium reached 1.68-12.29 mM and a percent confluency of 5% to 80% is achieved. In some embodiments, the undifferentiated pluripotent stem cells are cultured until the lactate concentration in the cell culture medium is between about 1.0 to 13. 0 mM, or between about 1.5-12.5 mM. In some embodiments, cells are cultured until the lactate concentration in the cell culture medium is between about 1.68-12.29 mM. In some embodiments, the percent confluence is between 5% and 90%, between 5% and 85%, or between 5% and 80%.

In some embodiments, the population of undifferentiated pluripotent stem cells are cultured a first cell culture medium comprising Bone morphogenetic protein 4 (BMP4) and 4-[4-(2H-1,3-Benzodioxol-5-yl)-5-(pyridin-2-yl)-1H-imidazol-2-yl]benzamide (SB431542). In some embodiments, the first cell culture medium comprises BMP4, SB431542, Fibroblast growth factor 2 (FGF2).

In some embodiments, the population of undifferentiated pluripotent stem cells (PSCs) are cultured in the first cell culture medium for at least 1 day, at least 2 days, at least 3 days, at least 5 days, at least 6 days, at least 7 days, at least 9 days, at least 11 days, at least 15 days, or at least 20 days, under conditions sufficient to produce differentiation into a target cell type, for example non-neuronal ectodermal (NNE) cells. In some embodiments, the population of PSCs are cultured in the first cell culture medium for at least 1 day. In some embodiments, the population of PSCs are cultured in the first cell culture medium for at least 4 days. In some embodiments, the population of PSCs are cultured in the first cell culture medium for at least 5 days. In some embodiments, the population of PSCs are cultured in the first cell culture medium for at least 7 days. In some embodiments, the population of PSCs are cultured in the first cell culture medium for between about 1-20 days, 1-9 days, 2-10 days, 3-7 days, or 4-6 days. In some embodiments, the population of PSCs are cultured in the first cell culture medium for between about 1-9 days. In some embodiments, the population of PSCs are cultured in the first cell culture medium for between about 3-7 days. In some embodiments, culturing the population of undifferentiated PSCs in the first cell culture medium produces a population of cells comprising non-neuronal ectodermal (NNE) cells.

In some embodiments, the population of cells produced by culturing the PSCs in the first cell culture medium, e.g., a population of cells comprising NNE cells, are cultured in a second cell culture medium comprising SB431542, Fibroblast growth factor 2 (FGF2), and N-(6-Methyl-2-benzothiazolyl)-2-[(3,4,6,7-tetrahydro-4-oxo-3-phenylthieno[3,2-d]pyrimidin-2-yl)thio]-acetamide (IWP-2) and 4-{6-[4-(Piperazin-1-yl)phenyl]pyrazolo[1,5-a]pyrimidin-3-yl}quinoline (LDN193189).

In some embodiments, the population of cells are cultured in the second cell culture medium for at least 1 day, at least 2 days, at least 3 days, at least 5 days, at least 6 days, at least 7 days, at least 9 days, at least 11 days, at least 15 days, or at least 20 days under conditions sufficient to produce differentiation into a target cell type, for example pre-placodal ectodermal (PPE) cells. In some embodiments, the population of cells are cultured in the second cell culture medium for at least 1 day. In some embodiments, the population of cells are cultured in the second cell culture medium for at least 4 days. In some embodiments, the population of cells are cultured in the second cell culture medium for at least 5 days. In some embodiments, the population of cells are cultured in the second cell culture medium for at least 6 days. In some embodiments, the population of cells are cultured in the second cell culture medium for at least 7 days. In some embodiments, the population of cells are cultured in the second cell culture medium for between about 1-20 days, 1-9 days, 2-10 days, 3-7 days, or 4-6 days. In some embodiments, the population of cells are cultured in the second cell culture medium for between about 1-9 days. In some embodiments, the population of cells are cultured in the second cell culture medium for between about 3-7 days. In some embodiments, the population of cells are cultured in the second cell culture medium for 1, 2, 3, 4, 5, 6, 7, 8, 9 or 10 days. In some embodiments, the population of cells are cultured in the second cell culture medium for about 5 days. In some embodiments, the population of cells are cultured in the second cell culture medium for 6 days. In some embodiments, the population of cells are cultured in the second cell culture medium for 7 days. In some embodiments, culturing the population of cells in the second cell culture medium produces a population of cells comprising PPE cells.

In some embodiments, the population of cells produced by culturing the cells in the second culture medium, e.g. the population of cells comprising PPE cells, are cultured in a third cell culture medium comprising 6-((2-((4-(2,4-Dichlorophenyl)-5-(4-methyl-1H-imidazol-2-yl)pyrimidin-2-yl)amino)ethyl)amino) nicotinonitrile (CHIR99021), FGF2, and Insulin-like growth factor 1 (IGF-1).

In some embodiments, the population of cells are cultured in the third cell culture medium for at least 1 day, at least 3 days, at least 5 days, at least 6 days, at least 7 days, at least 9 days, at least 11 days, at least 15 days, or at least 20 days, under conditions sufficient to produce differentiation into a target cell type, for example early otic neuronal progenitor (ONP) cells. In some embodiments, the population of cells are cultured in the third cell culture medium for at least 1 day. In some embodiments, the population of cells are cultured in the third cell culture medium for at least 4 days. In some embodiments, the population of cells are cultured in the third cell culture medium for at least 5 days. In some embodiments, the population of cells are cultured in the third cell culture medium for at least 7 days. In some embodiments, the population of cells are cultured in the third cell culture medium for 5 days. In some embodiments, the population of cells are cultured in the third cell culture medium for 7 days. In some embodiments, the population of cells are cultured in the third cell culture medium for 9 days. In some embodiments, the population of cells are cultured in the third cell culture medium for between about 1-20 days, 1-10 days, 1-17 days, 2-10 days, 3-7 days, or 4-6 days. In some embodiments, the population of cells are cultured in the third cell culture medium for between about 3-10 days. In some embodiments, the population of cells are cultured in the third cell culture medium for between about 5-9 days. In some embodiments, culturing the population of cells in the third cell culture medium produces a population of cells comprising early ONP cells.

In some embodiments, the population of cells produced by culturing the cells in the third culture medium, e.g. the population of cells comprising early ONP cells, are cultured in a fourth cell culture medium comprising Sonic Hedgehog (SHH), retinoic acid (RA), Epidermal growth factor (EGF), FGF2 and IGF-1.

In some embodiments, the population of cells are cultured in the fourth cell culture medium for at least 1 day, at least 3 days, at least 5 days, at least 7, days at least, 9 days, at least 11 days, or at least 15 days, or at least 20 days, under conditions sufficient to produce differentiation into a target cell type, for example mid-late ONP cells. In some embodiments, the population of cells are cultured in the fourth cell culture medium for at least 1 day. In some embodiments, the population of cells are cultured in the fourth cell culture medium for at least 4 days. In some embodiments, the population of cells are cultured in the fourth cell culture medium for at least 5 days. In some embodiments, the population of cells are cultured in the fourth cell culture medium for at least 7 days. In some embodiments, the population of cells are cultured in the fourth cell culture medium for 5 days. In some embodiments, the population of cells are cultured in the fourth cell culture medium for 7 days. In some embodiments, the population of cells are cultured in the fourth cell culture medium for 9 days. In some embodiments, the population of cells are cultured in the fourth cell culture medium for between about 1-20 days, 1-10 days, 1-17 days, 2-10 days, 3-7 days, or 4-6 days. In some embodiments, the population of cells are cultured in the fourth cell culture medium for between about 3-10 days. In some embodiments, the population of cells are cultured in the fourth cell culture medium for between about 5-9 days. In some embodiments, culturing the population of cells in the fourth cell culture medium produces a population of cells comprising mid-late ONP cells.

In some embodiments, the population of cells produced by culturing the cells in the third culture medium, e.g. the population of cells comprising mid-late ONP cells, are cultured in fifth cell culture medium comprising Brain derived neurotrophic factor (BDNF), Neurotrophin-3 (NT3), and IGF-1.

In some embodiments, the population of cells are cultured in the fifth cell culture medium for at least 1 day, at least 5 days, at least 10 days, at least 20 days, at least 40 days, at least 45 days, at least 50 days, at least 60 days, at least 70 days, at least 80 days, at least 90 days, or at least 100 days, under conditions sufficient to produce differentiation into a target cell type, for example late ONP cells. In some embodiments, the population of cells are cultured in the fifth cell culture medium for at least 1 day. In some embodiments, the population of cells are cultured in the fifth cell culture medium for at least 10 days. In some embodiments, the population of cells are cultured in the fifth cell culture medium for at least 20 days. In some embodiments, the population of cells are cultured in the fifth cell culture medium for at least 30 days. In some embodiments, the population of cells are cultured in the fifth cell culture medium for at least 45 days. In some embodiments, the population of cells are cultured in the fifth cell culture medium for at least 60 days. In some embodiments, the population of cells are cultured in the fifth cell culture medium for between about 1-65 days, 1-60 days, 1-50 days, 1-40 days, 1-20 days, 7-65 days, 5-50 days, 10-40 days, 10-30 days, 10-20 days, 20-60 days, 20-50 days, 20-45 days or 30-45 days. In some embodiments, the population of cells are cultured in the fifth cell culture medium for 7-65 days. In some embodiments, the population of cells are cultured in the fifth cell culture medium for 3-45 days. In some embodiments, the population of cells are cultured in the fifth cell culture medium for 10-60 days. In some embodiments, the population of cells are cultured in the fifth cell culture medium for 20-45 days. In some embodiments, culturing the population of cells in the fifth cell culture medium produces a population of cells comprising late ONP cells.

In some embodiments, culturing the population of cells in the culture medium comprises (i) harvesting the population of cells produced by culturing the cells in the fourth cell culture medium, e.g. a population of cells comprising mid-late ONP cells; (ii) seeding the population of cells in containers comprising the fifth cell culture medium; (iii) culturing the population of cells; (iv) harvesting the population of cells; (v) seeding the population of cells in containers comprising the fifth cell culture medium; and (vi) culturing the population of cells. In some embodiments the fifth cell culture medium further comprises a ROCK inhibitor. In some embodiments, the cells are cultured for between 5 and 35 days, between 7 and 35 days, between 7 and 30 days, or between 10 and 25 days at step (iii). In some embodiments, the cells are cultured for between 7 and 35 days at step (iii). In some embodiments, the cells are cultured for between 7 and 30 days, between 10 and 30 days, or between 15 and 25 days at step (vi). In some embodiments, the cells are cultured for between 7 and 30 days at step (vi). In some embodiments, the step (iii) described above comprises culturing the population of cells in containers comprising the fifth cell culture medium for at least 1 day, at least 5 days, at least 6 days, at least 7 days, at least 8 days, at least 10 days, at least 15 days, at least 20 days, at least 25 days, at least 30 days, at least 35 days, at least 40 days, at least 50 days, at least 60 days, or at least 70 days. In some embodiments, the step (vi) described above comprises culturing the population of cells for at least 1 day, at least 5 days, at least 6 days, at least 7 days, at least 8 days, at least 10 days, at least 15 days, at least 20 days, at least 25 days, at least 30 days, at least 35 days, at least 40 days, at least 50 days, or at least 60 days.

In some embodiments, the first cell culture medium includes BMP4 at a concentration of about 1 ng/mL, about 10 ng/ml, about 20 ng/mL, about 25 ng/ml, about 30 ng/ml, or about 40 ng/mL. In some embodiments, the first cell culture medium includes BMP4 at a concentration of between about 1 ng/mL to 40 ng/mL, 1 ng/mL to 25 ng/ml, 5 ng/ml to 30 ng/mL, or 10 ng/ml to 15 ng/mL. In some embodiments, the first cell culture medium includes BMP4 at a concentration of 1 ng/mL to 40 ng/mL. In some embodiments, the first cell culture medium includes BMP4 at a concentration of 1 ng/ml to 25 ng/mL. In some embodiments, the first cell culture medium includes BMP4 at a concentration of 5 ng/mL to 30 ng/mL. In some embodiments, the first cell culture medium includes BMP4 at a concentration of 10 ng/ml to 15 ng/ml. In some embodiments, the first cell culture medium includes BMP4 at a concentration of about 10 ng/mL. In some embodiments, the first cell culture medium includes BMP4 at a concentration of about 5 ng/mL. In some embodiments, the first cell culture medium includes BMP4 at a concentration of about 20 ng/mL.

In some embodiments, the first and/or second cell culture medium includes SB431542 at a concentration of about 0.1 μM, about 1 μM, about 5 μM, about 10 μM, about 15 μM, or about 20 μM. In some embodiments, the first and/or second cell culture medium includes SB431542 at a concentration of between about 0.1 μM-20 μM, 0.1-10 μM, 5 μM 15 μM, or 7 μM 13 μM. In some embodiments, the first and/or second cell culture medium includes SB431542 at a concentration of 1 μM-20 μM. In some embodiments, the first and/or second cell culture medium includes SB431542 at a concentration of 5 μM-15 μM. In some embodiments, the first and/or second cell culture medium includes SB431542 at a concentration of 0.1 μM-10 μM. In some embodiments, the first and/or second cell culture medium includes SB431542 at a concentration of about 1 μM.

In some embodiments, the first, second, third and/or fourth cell culture medium includes FGF2 in an amount of about 1 ng/mL, about 10 ng/mL, about 20 ng/ml, about 25 ng/mL, about 30 ng/mL, or about 40 ng/mL. In some embodiments, the first, second, third and/or fourth cell culture medium includes FGF2 in an amount of between about 1 ng/ml-40 ng/ml, 1 ng/ml-30 ng/mL, 1 ng/mL-25 ng/ml, 5 ng/mL-30 ng/ml, 5 ng/mL-15 ng/ml, or 10 ng/mL-15 ng/mL. In some embodiments, the first, second, third and/or fourth cell culture medium includes FGF2 in an amount of 1 ng/mL-40 ng/mL. In some embodiments, the first, second, third and/or fourth cell culture medium includes FGF2 in an amount of 1 ng/ml-25 ng/mL. In some embodiments, the first, second, third and/or fourth cell culture medium includes FGF2 in an amount of 10 ng/mL-15 ng/ml. In some embodiments, the first, second, third and/or fourth cell culture medium includes FGF2 in an amount of about 10 ng/mL.

In some embodiments, the second cell culture medium includes IWP-2 in an amount of about 0.5 μM, 1 μM, about 2 μM, about 3 μM, about 5 μM, or about 10 μM. In some embodiments, the second cell culture medium includes IWP-2 in an amount of between about 0.5 μM-20 μM, 0.5 μM-10 μM, 1 μM-10 μM, 1 μM-5 μM, 2 μM-7 μM, or 3 μM-5 μM. In some embodiments, the second cell culture medium includes IWP-2 in an amount of between about 0.5 μM-10 μM. In some embodiments, the second cell culture medium includes IWP-2 in an amount of about 2 μM-4 μM. In some embodiments, the second cell culture medium includes IWP-2 in an amount of about 2 μM.

In some embodiments, the second cell culture medium includes LDN193189 in an amount of about 10 nM, about 50 nM, about 100 nM, about 150 nM, about 200 nM, about 250 nM, about 300 nM, about 350 nM, about 400 nM, about 450 nM, about 500 nM, about 550 nM, or about 600 nM. In some embodiments, the second cell culture medium includes LDN193189 in an amount of between about 1 nM-600 nM, 50 nM-500 nM, 75 nM-200 nM, 100 nM-400 nM, or 200 nM-300 nM. In some embodiments, the second cell culture medium includes LDN193189 in an amount of 1 nM-600 nM. In some embodiments, the second cell culture medium includes LDN193189 in an amount of 50 nM-500 nM. In some embodiments, the second cell culture medium includes LDN193189 in an amount of 20 nM-400 nM. In some embodiments, the second cell culture medium includes LDN193189 in an amount of 75 nM-150 nM. In some embodiments, the second cell culture medium includes LDN193189 in an amount of about 100 nM.

In some embodiments, the third cell culture medium includes CHIR99021 at a concentration of about 1 μM, about 5 μM, about 6 μM, about 7 μM, about 10 μM, about 20 M, about 25 μM, about 30 μM, or about 40 μM. In some embodiments, the third cell culture medium includes CHIR99021 at a concentration of between about 1 μM-40 μM, 1 μM-30 μM, 1 μM-25 μM, 5 μM-20 μM, 2 μM-10 μM, or 5 μM-15 μM. In some embodiments, the third cell culture medium includes CHIR99021 at a concentration of 1 μM-40 μM. In some embodiments, the third cell culture medium includes CHIR99021 at a concentration of 1 μM-25 μM. In some embodiments, the third cell culture medium includes CHIR99021 at a concentration of 2 μM-8 μM. In some embodiments, the third cell culture medium includes CHIR99021 at a concentration of about 6 μM.

In some embodiments, the third, fourth and/or fifth cell culture medium includes IGF-1 in an amount of about 1 ng/mL, about 10 ng/mL, about 25 ng/mL, about 40 ng/mL, about 50 ng/ml, about 60 ng/mL, about 100 ng/mL, about 150 ng/mL, about 200 ng/mL, about 250 ng/ml, or about 300 ng/mL. In some embodiments, the third, fourth and/or fifth cell culture medium includes IGF-1 in an amount of 1 ng/mL-300 ng/ml, 20 ng/ml-200 ng/ml, 5 ng/mL-100 ng/ml, 25 ng/mL-300 ng/mL, or 40 ng/ml-100 ng/mL. In some embodiments, the third, fourth and/or fifth cell culture medium includes IGF-1 in an amount of 1 ng/ml-300 ng/mL. In some embodiments, the third, fourth and/or fifth cell culture medium includes IGF-1 in an amount of 25 ng/ml-300 ng/mL. In some embodiments, the third, fourth and/or fifth cell culture medium includes IGF-1 in an amount of 5 ng/mL-100 ng/mL. In some embodiments, the third, fourth and/or fifth cell culture medium includes IGF-1 in an amount of about 50 ng/ml.

In some embodiments, the fourth cell culture medium includes SHH in an amount of about 10 ng/ml, 30 ng/mL, about 50 ng/mL, about 100 ng/mL, about 300 ng/mL, about 500 ng/ml, about 600 ng/ml, about 700 ng/ml, about 800 ng/ml, about 900 ng/ml, about 1000 ng/ml, about 1100 ng/ml, about 1200 ng/ml, or about 1300 ng/mL. In some embodiments, the fourth cell culture medium includes SHH in an amount of 10 ng/mL-1300 ng/ml, 50 ng/ml-1000 ng/ml, 300 ng/ml-1000 ng/mL, or 400 ng/ml-600 ng/mL. In some embodiments, the fourth cell culture medium includes SHH in an amount of 10 ng/ml-1300 ng/mL. In some embodiments, the fourth cell culture medium includes SHH in an amount of 300 ng/ml-1000 ng/mL. In some embodiments, the fourth cell culture medium includes SHH in an amount of 400 ng/ml-600 ng/mL. In some embodiments, the fourth cell culture medium includes SHH in an amount of about 500 ng/mL.

In some embodiments, the fourth cell culture medium includes RA at a concentration of about 0.1 μM, about 0.2 μM, about 0.3 μM, about 0.5 μM, about 1 μM, about 2 μM, about 3 μM, about 5 μM, about 10 μM, about 15 μM, or about 20 μM. In some embodiments, the fourth culture medium includes RA at a concentration of 0.1 μM-20 μM, 0.1 μM-5 μM, 0.5 μM-5 μM, 0.2 μM-5 μM, or 0.5 μM-2 μM. In some embodiments, the fourth cell culture medium includes RA at a concentration of 0.1 μM-20 μM. In some embodiments, the fourth cell culture medium includes RA at a concentration of 0.2 μM-5 μM. In some embodiments, the fourth cell culture medium includes RA at a concentration of 0.2 μM-2 μM. In some embodiments, the fourth cell culture medium includes RA at a concentration of about 0.5 μM.

In some embodiments, the fourth cell culture medium includes EGF in an amount of about 5 ng/mL, about 10 ng/ml, about 15 ng/ml, about 20 ng/mL, about 25 ng/mL, about 50 ng/ml, about 100 ng/mL, about 150 ng/mL, about 200 ng/ml, about 250 ng/ml, or about 300 ng/ml. In some embodiments, the fourth cell culture medium includes EGF in an amount of 1 ng/mL-300 ng/mL, 10 ng/ml-200 ng/mL, 20 ng/mL-300 ng/mL, 5 ng/ml-100 ng/mL, or 10 ng/ml-50 ng/mL. In some embodiments, the fourth cell culture medium includes EGF in an amount of 1 ng/mL-300 ng/mL. In some embodiments, the fourth cell culture medium includes EGF in an amount of 5 ng/mL-100 ng/mL. In some embodiments, the fourth cell culture medium includes EGF in an amount of 10 ng/ml-50 ng/mL. In some embodiments, the fourth cell culture medium includes EGF in an amount of about 20 ng/mL.

In some embodiments, the fifth cell culture medium includes BDNF in an amount of about 5 ng/mL, about 10 ng/ml, about 15 ng/ml, about 25 ng/ml, about 50 ng/ml, about 100 ng/mL, about 150 ng/mL, about 200 ng/mL, about 250 ng/mL, or about 300 ng/mL. In some embodiments, the fifth cell culture medium includes BDNF in an amount of 1 ng/ml-300 ng/ml, 5 ng/mL-200 ng/mL, 5 ng/mL-100 ng/mL, or 5 ng/mL-50 ng/mL. In some embodiments, the fifth cell culture medium includes BDNF in an amount of 1 ng/mL-300 ng/mL. In some embodiments, the fifth cell culture medium includes BDNF in an amount of 5 ng/ml-100 ng/mL. In some embodiments, the fifth cell culture medium includes BDNF in an amount of 5 ng/ml-30 ng/ml. In some embodiments, the fifth cell culture medium includes BDNF in an amount of about 10 ng/mL.

In some embodiments, the fifth cell culture medium comprises neurotrophin 3 (NT3, also referred to as NT-3, and NTF3). NT3 is a member of the neurotrophin family, which is involved in the survival and differentiation of mammalian neurons. NT3 is thought to be involved in the maintenance of the adult nervous system, and development of neurons in the embryo. In some embodiments, the fifth cell culture medium includes NT3 in an amount of about 5 ng/ml, about 10 ng/mL, about 15 ng/mL, about 25 ng/mL, about 50 ng/mL, about 100 ng/mL, about 150 ng/ml, about 200 ng/ml, about 250 ng/mL, or about 300 ng/mL. In some embodiments, the fifth cell culture medium includes NT3 in an amount of 1 ng/mL-300 ng/mL, 5 ng/ml-200 ng/ml, 5 ng/mL-100 ng/mL, or 5 ng/mL-50 ng/mL. In some embodiments, the fifth cell culture medium includes NT3 in an amount of 1 ng/mL-300 ng/mL. In some embodiments, the fifth cell culture medium includes NT3 in an amount of 5 ng/mL-100 ng/mL. In some embodiments, the fifth cell culture medium includes NT3 in an amount of 5 ng/ml-30 ng/mL. In some embodiments, the fifth cell culture medium comprises NT3 in an amount of 10 ng/ml.

In some embodiments, the fifth cell culture medium comprises a Rock inhibitor, e.g. a small molecule inhibitor that inhibits ROCK1 and/or ROCK2 mediated signaling. In some embodiments, the Rock inhibitor comprises Y-27632 dihydrochloride (trans-4-[(1R)-1-Aminoethyl]-N-4-pyridinylcyclohexanecarboxamide dihydrochloride). In some embodiments, the fifth cell culture medium comprises a Rock inhibitor in an amount of about 0.5 μM, 1.0 μM, 1.5 μM, 2.0 μM, 3.0 μM, 5 μM, 7 μM, 9 μM, 10 μM, 11 μM, 12 μM, 15 μM, 20 μM, 30 μM, 40 M, 50 μM, or 60 μM. In some embodiments, the fifth cell culture medium comprises a Rock inhibitor in an amount of between about 0.5 μM-60 μM, 1 μM-50 μM, 2 μM-50 μM, 1 μM-30 μM, 2-μM, 5 μM-20μ, 1 μM-15 μM or 5 μM-15μ. In some embodiments, the fifth cell culture medium comprises a Rock inhibitor in an amount of between about 2 μM-50 μM. In some embodiments, the fifth cell culture medium comprises a Rock inhibitor in an amount of about 10 μM.

In some embodiments, the methods comprise (a) culturing a population of undifferentiated pluripotent stem cells in a first cell culture medium comprising FGF2 at a concentration of between 1-25 ng/mL, BMP4 at a concentration of between 1-25 ng/ml and SB431542 at a concentration of between 0.1-10 μM for 1-9 days under conditions sufficient to produce non-neuronal ectodermal (NNE) cells, thereby producing a population of cells comprising NNE cells; (b) culturing the population of cells comprising NNE cells in a second cell culture medium comprising SB431542 at a concentration of between 0.1-10 μM, FGF2 at a concentration of between 1-25 ng/ml, IWP-2 at a concentration of between 0.5 10 μM and LDN193189 at a concentration of between 20-400 nM for 1-9 days under conditions sufficient to produce pre-placodal ectodermal (PPE) cells, thereby producing a population of cells comprising PPE cells; a step (c) culturing the population of cells comprising PPE cells in a third cell culture medium comprisingCHIR99021 at a concentration of between 1-25 μM, FGF2 at a concentration of between 1-25 ng/ml, and IGF-1 at a concentration of between 5-100 ng/mL for 5-9 days under conditions sufficient to produce early Otic Neuronal progenitor (ONP) cells, thereby producing a population of cells comprising early ONP cells; (d) culturing the population of cells comprising early ONP cells in a fourth cell culture medium comprising SHH at a concentration of between 50-1000 ng/mL, RA at a concentration of between 0.2-2 μM, EGF at a concentration of between 5-100 ng/mL, FGF2 at a concentration of between 1-25 ng/ml and IGF-1 at a concentration of between 5-100 ng/ml for 5-9 days under conditions sufficient to produce mid-late ONP cells, thereby producing a population of cells comprising mid-late ONP cells; (e) culturing the population of cells comprising mid-late ONP cells in a fifth cell culture medium comprising BDNF at a concentration of between 5-100 ng/mL, NT3 at a concentration of between 5-100 ng/ml, and IGF-1 at a concentration of between 5-100 ng/mL for 3-45 days under conditions sufficient to produce late ONP cells, thereby producing a population of cells comprising late ONP cells; and (f) collecting the population of cells comprising late ONPs, thereby producing the composition. In alternative embodiments, the first cell culture medium at step (a) comprises BMP4 at a concentration of between 1-25 ng/ml and SB431542 at a concentration of between 0.1-10 μM, and does not comprise FGF2.

In some embodiments, the methods comprise (a) culturing a population of undifferentiated pluripotent stem cells in a first cell culture medium comprising BMP4 and SB431542 and FGF2 for 1-9 days under conditions sufficient to produce non-neuronal ectodermal (NNE) cells, thereby producing a population of cells comprising NNE cells; (b) culturing the population of cells comprising NNE cells in a second cell culture medium comprising SB431542, FGF2, and IWP-2 and LDN193189 for 1-9 days under conditions sufficient to produce pre-placodal ectodermal (PPE) cells, thereby producing a population of cells comprising PPE cells; a step (c) culturing the population of cells comprising PPE cells in a third cell culture medium comprising CHIR99021, FGF2, and IGF-1 for 5-9 days under conditions sufficient to produce early Otic Neuronal progenitor (ONP) cells, thereby producing a population of cells comprising early ONP cells; (d) culturing the population of cells comprising early ONP cells in a fourth cell culture medium comprising SHH, RA, EGF, FGF2 and IGF-1 for 5-9 days under conditions sufficient to produce mid-late ONP cells, thereby producing a population of cells comprising mid-late ONP cells; (e) culturing the population of cells comprising mid-late ONP cells in a fifth cell culture medium comprising BDNF, NT3, and IGF-1 for 3-45 days under conditions sufficient to produce late ONP cells, thereby producing a population of cells comprising late ONP cells; and (f) collecting the population of cells comprising late ONPs, thereby producing the composition. In alternative embodiments, the first cell culture medium at step (a) comprises BMP4 and SB431542, and does not comprise FGF2.

In some embodiments, the methods comprise (a) culturing a population of undifferentiated pluripotent stem cells in a first cell culture medium comprising FGF2 at a concentration of between 1-25 ng/mL, BMP4 at a concentration of between 1-25 ng/ml and SB431542 at a concentration of between 0.1-10 μM under conditions sufficient to produce non-neuronal ectodermal (NNE) cells, thereby producing a population of cells comprising NNE cells; (b) culturing the population of cells comprising NNE cells in a second cell culture medium comprising SB431542 at a concentration of between 0.1-10 μM, FGF2 at a concentration of between 1-25 ng/mL, and IWP-2 at a concentration of between 0.5-10 μM and LDN193189 at a concentration of between 20-400 nM under conditions sufficient to produce pre-placodal ectodermal (PPE) cells, thereby producing a population of cells comprising PPE cells; (c) culturing the population of cells comprising PPE cells in a third cell culture medium comprising CHIR99021 at a concentration of between 1-25 μM, FGF2 at a concentration of between 1-25 ng/ml, and IGF-1 is at a concentration of between 5-100 ng/mL under conditions sufficient to produce early Otic Neuronal progenitor (ONP) cells, thereby producing a population of cells comprising early ONP cells; (d) culturing the population of cells comprising early ONP cells in a fourth cell culture medium comprising SHH at a concentration of between 50-1000 ng/mL, RA at a concentration of between 0.2-2 μM, EGF at a concentration of between 5-100 ng/mL, FGF2 at a concentration of between 1-25 ng/mL and IGF-1 at a concentration of between 5-100 ng/ml under conditions sufficient to produce mid-late ONP cells, thereby producing a population of cells comprising mid-late ONP cells; a step (e) culturing the population of cells comprising mid-late ONP cells in a fifth cell culture medium comprising BDNF at a concentration of between 5-100 ng/ml, NT3 at a concentration of between 5-100 ng/mL, and IGF-1 at a concentration of between 5-100 ng/ml under conditions sufficient to produce late ONP cells, thereby producing a population of cells comprising late ONP cells; and (f) collecting the population of cells comprising late ONPs, thereby producing the composition. In alternative embodiments, the first cell culture medium at step (a) comprises BMP4 at a concentration of between 1-25 ng/mL and SB431542 at a concentration of between 0.1-10 μM, and does not comprise FGF2.

In some embodiments, the method comprises (a) culturing a population of undifferentiated pluripotent stem cells in a first cell culture medium comprising FGF2 at a concentration of 10 ng/mL, BMP4 at a concentration of 10 ng/mL and SB431542 at a concentration of 1 μM for 3-7 days under conditions sufficient to produce non-neuronal ectodermal (NNE) cells, thereby producing a population of cells comprising NNE cells; (b) culturing the population of cells comprising NNE cells in a second cell culture medium comprising SB431542 at a concentration of 1 μM, FGF2 at a concentration of 10 ng/mL, and IWP-2 at a concentration of between 2 μM and LDN193189 at a concentration of 100 nM for 3-7 days under conditions sufficient to produce pre-placodal ectodermal (PPE) cells, thereby producing a population of cells comprising PPE cells; (c) culturing the population of cells comprising PPE cells in a third cell culture medium comprising CHIR99021 at a concentration of 6 μM, FGF2 at a concentration of 10 ng/ml, and IGF-1 is at a concentration of 50 ng/mL for 7 days under conditions sufficient to produce early Otic Neuronal progenitor (ONP) cells, thereby producing a population of cells comprising early ONP cells; (d) culturing the population of cells comprising early ONP cells in a fourth cell culture medium comprising SHH at a concentration of 500 ng/mL, RA at a concentration of 0.5 μM, EGF at a concentration of 20 ng/mL, FGF2 at a concentration of 10 ng/mL and IGF-1 at a concentration of 50 ng/ml for 7 days under conditions sufficient to produce mid-late ONP cells, thereby producing a population of cells comprising mid-late ONP cells; (e) culturing the population of cells comprising mid-late ONP cells in a fifth cell culture medium comprising BDNF at a concentration of 10 ng/mL, NT3 at a concentration of 10 ng/ml, and IGF-1 at a concentration of 50 ng/ml for 3-45 days under conditions sufficient to produce late ONP cells, thereby producing a population of cells comprising late ONP cells; and a step (f) collecting the population of cells comprising late ONPs, thereby producing the composition. In alternative embodiments, the first cell culture medium at step (a) comprises BMP4 at a concentration of 10 ng/ml and SB431542 at a concentration of 1 μM, and does not comprise FGF2.

In some embodiments, any one of the methods described above comprises, prior to step (a), seeding the undifferentiated pluripotent stem cells at a density of 1,200-20,000 live cells/cm² in a monolayer, and culturing the cells to until a lactate concentration in the cell culture medium reached 1.68-12.29 mM and a percent confluency of 5-80% was achieved.

In some embodiments, any one of the methods described above further comprises step of cryopreserving the population of cells comprising late ONPs.

The skilled artisan will understand that at any of the steps described above, the resultant population of cells can be cryopreserved, followed by seeding and culture in the culture medium appropriate for the next stage of the differentiation process.

### Methods of Treatment

Methods for treating a subject who has, or who is at risk for developing, an auditory disorder, are also described and are within the scope of the present invention. Auditory disorders include but are not intended to be limited to conductive hearing loss, sensorineural hearing loss, mixed hearing loss, auditory neuropathy spectrum disorder, central hearing loss, central auditory processing disorder and tinnitus. These methods include administering a cell or population of cells as described herein to the ear of the subject. The administered cells may be obtained by the methods described herein, and the starting material may be tissue obtained from the subject to be treated. In other embodiments, the methods include the step of administering a therapeutic agent that promotes the expression of an auditory protein within a cell within the inner ear (e.g., a differentiation agent as described herein). When used, the differentiation agent can be administered to cells in culture or can be administered to the subject either alone (to stimulate the differentiation of stem cells or progenitor cells within the subject's inner ear) or together with undifferentiated cells (e.g., undifferentiated cells isolated by the methods described herein). The differentiation agent can be, for example, an agonist of the hedgehog pathway, such as an agonist of Sonic hedgehog or Purmorphamin (e.g., Hh-Ag1.3).

A subject having a disorder of the inner ear, or at risk for developing such a disorder, can be treated with the auditory cells as described herein. In a successful engraftment, at least some transplanted spiral ganglion neurons, for example, will form synaptic contacts with hair cells and with targets in the cochlear nucleus. To improve the ability of the cells to engraft, the stem cells can be modified prior to differentiation. For example, the cells can be engineered to overexpress one or more anti-apoptotic genes in the progenitor or differentiated cells. The Fak tyrosine kinase or Akt genes are candidate anti-apoptotic genes that can be useful for this purpose; overexpression of FAK or Akt can prevent cell death in spiral ganglion cells and encourage engraftment when transplanted into another tissue, such as an explanted organ of Corti (see for example, Mangi et al., Nat. Med. 9:1195-201, 2003). Neural progenitor cells overexpressing alpha.sub.v.beta.sub.3 integrin may have an enhanced ability to extend neurites into a tissue explant, as the integrin has been shown to mediate neurite extension from spiral ganglion neurons on laminin substrates (Aletsee et al., Audiol. Neurootol. 6:57-65, 2001). In another example, ephrinB2 and ephrinB3 expression can be altered, such as by silencing with RNAi or overexpression with an exogenously expressed cDNA, to modify EphA4 signaling events. Spiral ganglion neurons have been shown to be guided by signals from EphA4 that are mediated by cell surface expression of ephrin-B2 and -B3 (Brors et al., J. Comp. Neurol. 462:90-100, 2003). Inactivation of this guidance signal may enhance the number of neurons that reach their target in an adult inner ear. Exogenous factors such as the neurotrophins BDNF and NT3, and LIF can be added to tissue transplants to enhance the extension of neurites and their growth towards a target tissue in vivo and in ex vivo tissue cultures. Neurite extension of sensory neurons can be enhanced by the addition of neurotrophins (BDNF, NT3) and LIF (Gillespie et al., NeuroReport 12:275-279, 2001). A Sonic hedgehog (Shh) polypeptide or polypeptide fragment (e.g., SHH-N), can also be useful as an endogenous factor to enhance neurite extension. Shh is a developmental modulator for the inner ear and a chemoattractant for axons (Charron et al., Cell 113:11 23, 2003).

A subject experiencing or at risk for developing a hearing loss is a candidate for the treatment methods described herein. For example, the subject can receive a transplant of inner ear hair cells or spiral ganglion cells generated by exposure to a differentiation agent, or the subject can be administered an agent identified as being capable of causing a stem cell to differentiate into a cell of the inner ear. A subject having or at risk for developing a hearing loss can hear less well than the average subject being, or less well than a subject before experiencing the hearing loss. For example, hearing can be diminished by at least 5, 10, 30, 50% or more. The subject can have sensorineural hearing loss, which results from damage or malfunction of the sensory part (the cochlea) or the neural part (the auditory nerve) of the ear, or conductive hearing loss, which is caused by blockage or damage in the outer and/or middle ear, or the subject can have mixed hearing loss, which is caused by a problem in both the conductive pathway (in the outer or middle ear) and in the nerve pathway (the inner ear). An example of a mixed hearing loss is a conductive loss due to a middle-ear infection combined with a sensorineural loss due to damage associated with aging.

The subject can be deaf or have a hearing loss for any reason or as a result of any type of event. For example, a subject can be deaf because of a genetic or congenital defect; for example, a subject can have been deaf since birth, or can be deaf or hard-of-hearing as a result of a gradual loss of hearing due to a genetic or congenital defect. In another example, a subject can be deaf or hard-of-hearing as a result of a traumatic event, such as a physical trauma to a structure of the ear, or a sudden loud noise, or a prolonged exposure to loud noises. For example, prolonged exposures to concert venues, airport runways, and construction areas can cause inner ear damage and subsequent hearing loss. A subject can experience chemical-induced ototoxicity, wherein ototoxins include therapeutic drugs including antineoplastic agents, salicylates, quinines, and aminoglycoside antibiotics, contaminants in foods or medicinals, and environmental or industrial pollutants. A subject can have a hearing disorder that results from aging, or the subject can have tinnitus (characterized by ringing in the ears).

A subject suitable for the pharmaceutical compositions and methods as described herein can include a subject having a vestibular dysfunction, including bilateral and unilateral vestibular dysfunction. Vestibular dysfunction is an inner ear dysfunction characterized by symptoms that include dizziness, imbalance, vertigo, nausea, and fuzzy vision and may be accompanied by hearing problems, fatigue and changes in cognitive functioning. Vestibular dysfunction can be the result of a genetic or congenital defect; an infection, such as a viral or bacterial infection; or an injury, such as a traumatic or nontraumatic injury. Vestibular dysfunction is most commonly tested by measuring individual symptoms of the disorder (e.g., vertigo, nausea, and fuzzy vision).

The compositions and methods as described herein may be used for the treatment of hearing disorders resulting from sensorineural hair cell loss or auditory neuropathy. Subjects suffering from auditory neuropathy experience a loss of cochlear sensory neurons while the hair cells of the inner ear remain intact. Such subjects will benefit particularly from treatment that causes cells (stem cells or progenitor cells) to differentiate into spiral ganglion cells, or from administration of spiral ganglion cells into the inner ear. Subjects with sensorineural hair cell loss experience the degeneration of cochlear hair cells, which frequently results in the loss of spiral ganglion neurons in regions of hair cell loss. Such subjects may also experience loss of supporting cells in the organ of Corti, and degeneration of the limbus, spiral ligament, and stria vascularis in the temporal bone material. Such subjects can receive treatment with an agent that causes cells to differentiate into hair cells, or a tissue transplant containing hair cells grafted or injected into the inner ear. The subjects may additionally benefit from treatment that causes cells to differentiate into spiral ganglion cells, or from administration of spiral ganglion cells into the inner ear. For example, in auditory nerve degeneration from mechanical compression, most auditory spiral ganglion cells degenerate (causing trans neuronal death of the cochlear nucleus cells) following sustained compression in the Rosenthal's canal, together with astocytes and Schwann cell columns form a continuous, “naturally occurring autologous cell bridge”, which acts as an anatomical scaffold for grafted cells migration to connect between the PNS and the CNS (Sekiya et al. 2021 Cell Transplantation Volume 30:1-20).

In some embodiments, the methods provided herein are methods for replacing auditory neurons in a subject in need thereof. In some embodiments, the methods provided herein are methods for augmenting an existing but damaged auditory neuron population in a subject in need thereof.

Auditory cells generated by the methods described herein can be administered, such as in the form of a cell suspension, into, on to or near, for example, the inner ear or the middle ear, by injection, such as into the luminae of the cochlea or the auditory nerve through the retromastoid route. Injection can be, for example, through the round window of the ear or through the bony capsule surrounding the cochlea. The cells can be injected through the round window into the auditory nerve trunk in the internal auditory meatus or into the Scala tympani, as described below. The administration of the auditory cells as described herein can be accomplished with, for example, injection needle or syringe positioning devices known in the art that have the ability to control (e.g., either manually or through a robotic interface) the navigation and position of a needle to the desired target anatomy of, for example, the inner ear or middle ear, for the treatment of auditory or hearing loss conditions as described herein. Such devices include, for example the stabilization that is required to facilitate safe and effective delivery of the auditory cells over a period of time to ensure delivery of the concentration or volume of auditory cells as described herein. Exemplary routes of administration are described at, for example, otosurgeryatlas.stanford.edu/otologic-surgery-atlas/cochlear-implantation/cochlear-implant-surgical-variations/.

In an exemplary route of administration, for example to the Scala tympani by cochleostomy, a small hole is drilled through the Otic capsule in the base of cochlea to accommodate a cannula. The hole is covered with a small piece of fascia. Cells are loaded into a 30G cannula, primed with saline and aspirated with about 1 μL air, followed by aspiration of the composition. The cells are injected into the Scala tympani using a pump, at a rate of about 1 μL/minute. In a second exemplary route of administration, the cochleostomy is through the otic capsule. A dental drill is used to create a hole to accommodate a 33G need and cannula into the modiolus, and then through the bony wall of the modiolus via the hole puncture. The hole is covered with a small piece of fascia. Cells are loaded into the cannula, and injected into the modiolus, as described above. The cannula is left in place for about 10 minutes to allow the fluids to equilibrate. Alternatively, compositions of the disclosure can be delivered through the round window. First, the round window is exposed, followed by removal or incision of the round window mucosa membrane and drilling away of the boney overhang. A cannula is directed through the round window into the Scala tympani or modiolus, and cells are administered as described above.

Accordingly, the disclosure provides methods of treating an auditory condition, comprising administering to a therapeutically effective amount of a pharmaceutical composition comprising a population of auditory cells, wherein: (a) greater than or equal to 20% of the cells in the population express SOX2; (b) greater than or equal to 10% of the cells in the population express β tubulin III; (c) greater than or equal to 5% of the cells in the population express TrkB; and (d) less than or equal to 1% of the cells in the population express TRA-1-60 and/or SSEA5; wherein the composition is administered to the inner or middle ear of the subject. In some embodiments, the method comprises administering a pharmaceutical composition wherein (a) greater than or equal to 30% of the cells in the population express SOX2; (b) greater than or equal to 30% of the cells in the population express PAX2; (c) greater than or equal to 30% of the cells in the population express β tubulin III; (d) greater than or equal to 20% of the cells in the population express TrkB; (e) greater than or equal to 30% of the cells in the population express GluA4; (f) less than or equal to 20% of the cells in the population express Myo7A; and (d) less than or equal to 0.1% of the cells in the population express TRA-1-60 and/or SSEA5.

In some embodiments, the method comprises administering between about 100,000 and 50 million cells, between about 100,000 and 10 million cells, between about 100,000 and 1 million cells, between about 200,000 and 10 million cells, between about 500,000 and 1 million cells, or between about 100,000 and 500,000 cells to the subject.

In some embodiments, administration of the compositions described herein alleviates a sign or symptom of the auditory disease or condition in the subject. In some embodiments, the method improves hearing in the subject, lessens the severity of hearing loss, delays the progression of hearing loss, alleviating one or more symptoms associated with the hearing disease or disorder.

The administration of the auditory cells as described herein can be accomplished with, for example, by pre-injection of a coating material, such as a matrix component, serum component, or biodegradable scaffold that may enhance the attachment and integration of the transplanted sensory neurons, to ensure delivery of the concentration or volume of auditory cells as described herein. The cell product can be cryopreserved in a cryovial, made of plastic, glass, or other polymers or rubber and plastic copolymers such as Cyclic Olefin Copolymer. The vials can be seals with a screw cap or a stopper made of a rubber and plastic copolymers such as Thermo Plastic Elastomers that enable sterile transfer of the cell product into the delivery device. The cell product can be cryopreserved preloaded within a syringe, a syringe cartridge, or an injection canula, which is thawed prior to administration to the subject.

### Pharmaceutical Compositions

The disclosure provides pharmaceutical compositions comprising a population of cells expressing the markers described herein. The skilled artisan will appreciate that the compositions described herein can comprise mixed populations of cell types, whose identities are reflected in percentages of cells in the percentages cells in the population expressing one or more of the markers described herein. Individual cells in the population may express only a single marker described below, or individual cells may express combinations of markers described below, depending on the differentiation state of the cell. Cells in the population may express neural progenitor markers such as Nestin ONP markers such as PAX2, PAX8, and/or SOX2, neuronal markers such as β Tubulin III, auditory neuron markers such as TrkB and/or GluA4, non-specific neuronal markers such as Myo7A, or hESC markers such as TRA-1-60 and/or SSEA5.

In some embodiments, cells in the population express SOX2; cells in the population express β tubulin III; cells in the population express TrkB; and cells in the population express TRA-1-60 and/or SSEA5.

In some embodiments, cells in the population express SOX2; cells in the population express PAX2; cells in the population express β tubulin III; cells in the population express TrkB; cells in the population express GluA4; cells in the population express Myo7A; and optionally cells in the population express TRA-1-60 and/or SSEA5.

In some embodiments, cells in the population express Nestin. Nestin is member of the intermediate filament protein family and is expressed in neurons. In some embodiments, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 95%, or greater than or equal to 99% of the cells in the population express Nestin. In some embodiments, greater than or equal to 40% of the cells in the population express Nestin. In some embodiments, greater than or equal to 50% of the cells in the population express Nestin. In some embodiments, greater than or equal to 60% of the cells in the population express Nestin. In some embodiments, greater than or equal to 70% of the cells in the population express Nestin. In some embodiments, greater than or equal to 80% of the cells in the population express Nestin. In some embodiments, greater than or equal to 90% of the cells in the population express Nestin. In some embodiments, 10% to 95%, 20% to 90%, 30% to 80%, 40% to 70%, or 30% to 60% of the cells in the population express Nestin.

In some embodiments, cells in the population express SOX2. SOX2 encodes a member of the SRY-related HMG-box (SOX) family of transcription factors involved in the regulation of embryonic development and in the determination of cell fate. In some embodiments, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 95%, or greater than or equal to 99% of the cells in the population express SOX2. In some embodiments, greater than or equal to 30% of the cells in the population express SOX2. In some embodiments, greater than or equal to 40% of the cells in the population express SOX2. In some embodiments, greater than or equal to 50% of the cells in the population express SOX2. In some embodiments, greater than or equal to 60% of the cells in the population express SOX2. In some embodiments, greater than or equal to 70% of the cells in the population express SOX2. In some embodiments, greater than or equal to 80% of the cells in the population express SOX2. In some embodiments, greater than or equal to 90% of the cells in the population express SOX2. In some embodiments, 10% to 95%, 20% to 90%, 30% to 80%, 40% to 70%, or 30% to 60% of the cells in the population express SOX2.

In some embodiments, cells in the population express PAX8. PAX8 encodes a member of the paired box family of transcription factors containing a paired box domain, an octapeptide, and a paired-type homeodomain domain. In some embodiments, less than 70%, less than 60%, less than 50%, less than 40%, %, less than 20%, less than 10%, or less than 5%, of the cells in the population express PAX8. In some embodiments, less than 60% of the cells in the population express PAX8. In some embodiments, less than 50% of the cells in the population express PAX8. In some embodiments, less than 40% of the cells in the population express PAX8. In some embodiments, less than 20% of the cells in the population express PAX8. In some embodiments, less than 10% of the cells in the population express PAX8. In some embodiments, less than 5% of the cells in the population express PAX8. In some embodiments, less than 1% of the cells in the population express PAX8. In some embodiments, 0.1% to 60%, 1% to 50%, 0.1% to 30%, 1% to 20%, 5% to 15%, or 5% to 8% of the cells in the population express PAX8.

In some embodiments, cells in the population express Six1. Six1 is a homeobox protein gene found in a cluster of related genes on chromosome 14 and is thought to be involved in limb development. In some embodiments, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 95%, or greater than or equal to 99% of the cells in the population express Six1. In some embodiments, greater than or equal to 20% of the cells in the population express Six1. In some embodiments, greater than or equal to 30% of the cells in the population express Six1. In some embodiments, greater than or equal to 40% of the cells in the population express Six1. In some embodiments, greater than or equal to 50% of the cells in the population express Six1. In some embodiments, greater than or equal to 60% of the cells in the population express Six1. In some embodiments, greater than or equal to 70% of the cells in the population express Six1. In some embodiments, greater than or equal to 80% of the cells in the population express Six1. In some embodiments, 10% to 95%, 20% to 90%, 30% to 80%, 40% to 70%, or 30% to 60% of the cells in the population express Six1.

In some embodiments, cells in the population express PAX2. PAX2 encodes paired box gene 2 is a target of transcriptional suppression by the tumor suppressor gene WT1. In some embodiments, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 95%, or greater than or equal to 99% of the cells in the population express PAX2. In some embodiments, greater than or equal to 20% of the cells in the population express PAX2. In some embodiments, greater than or equal to 30% of the cells in the population express PAX2. In some embodiments, greater than or equal to 40% of the cells in the population express PAX2. In some embodiments, greater than or equal to 50% of the cells in the population express PAX2. In some embodiments, greater than or equal to 60% of the cells in the population express PAX2. In some embodiments, greater than or equal to 70% of the cells in the population express PAX2. In some embodiments, greater than or equal to 80% of the cells in the population express PAX2. In some embodiments, greater than or equal to 90% of the cells in the population express PAX2. In some embodiments, 10% to 99%, 20% to 95%, 30% to 80%, 40% to 70%, or 50% to 60% of the cells in the population express PAX2.

In some embodiments, cells in the population express GluA4. GluA4 encodes a glutamate receptor expressed in excitatory neurotransmitter secreting neurons in the brain and are activated in a variety of normal neurophysiologic processes. In some embodiments, greater than or equal to 1%, greater than or equal to 5%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 95%, or greater than or equal to 99% of the cells in the population express GluA4. In some embodiments, greater than or equal to 10% of the cells in the population express GluA4. In some embodiments, greater than or equal to 20% of the cells in the population express GluA4. In some embodiments, greater than or equal to 30% of the cells in the population express GluA4. In some embodiments, greater than or equal to 40% of the cells in the population express GluA4. In some embodiments, greater than or equal to 50% of the cells in the population express GluA4. In some embodiments, greater than or equal to 70% of the cells in the population express GluA4. In some embodiments, greater than or equal to 90% of the cells in the population express GluA4. In some embodiments, 1% to 99%, 10% to 95%, 20% to 90%, 30% to 80%, 30% to 60%, or 20% to 50% of the cells in the population express GluA4. In some embodiments, between about 10% to 95% of the cells in the population express GluA4. In some embodiments, 30% to 90 of the cells in the population express GluA4.

In some embodiments, cells in the population express CD133. CD133 encodes a pentaspan transmembrane glycoprotein that localizes to membrane protrusions and is often expressed on adult stem cells where it functions in maintaining stem cell properties by suppressing differentiation. In some embodiments, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to greater than or equal to 95%, or greater than or equal to 99% of the cells in the population express CD133. In some embodiments, greater than or equal to 50% of the cells in the population express CD133. In some embodiments, greater than or equal to 60% of the cells in the population express CD133. In some embodiments, greater than or equal to 70% of the cells in the population express CD133. In some embodiments, greater than or equal to 80% of the cells in the population express CD133. In some embodiments, greater than or equal to 90% of the cells in the population express CD133. In some embodiments, greater than or equal to 95% of the cells in the population express CD133. In some embodiments, 1% to 99% 10% to 95%, 20% to 90%, 30% to 80%, or 40% to 70%, of the cells in the population express CD133.

In some embodiments, cells in the population express GATA3. GATA3 is a regulator of T-cell development and plays a role in endothelial cell biology. Defects in GATA3 are the cause of hypoparathyroidism with sensorineural deafness. In some embodiments, less than 10%, less than 5%, less than 1%, less than 0.1% of the cells in the population express GATA3. In some embodiments, less than 5% of the cells in the population express GATA3. In some embodiments, less than 1% of the cells in the population express GATA3. In some embodiments, less than 0.1% of the cells in the population express GATA3. In some embodiments, 0.1% to 10%, 0.1% to 5%, or 0.1% to 1% of the cells in the population express GATA3.

In some embodiments, cells in the population express β tubulin III (also referred to as β III tubulin or Beta 3 tubulin and the like). β tubulin III encodes a member of the beta tubulin protein family that heterodimerize and assemble to form microtubules. In some embodiments, greater than or equal to greater than or equal to 5%, greater than or equal to 10%, greater than or equal to 20%, greater than or equal to 30%, greater than or equal to 40%, greater than or equal to 50%, greater than or equal to 60%, greater than or equal to 70%, or greater than or equal to 80% of the cells in the population express B tubulin III. In some embodiments, greater than or equal to 10% of the cells in the population express β tubulin III. In some embodiments, greater than or equal to 20% of the cells in the population express β tubulin III. In some embodiments, greater than or equal to 30% of the cells in the population express β tubulin III. In some embodiments, greater than or equal to 40% of the cells in the population express β tubulin III. In some embodiments, greater than or equal to 50% of the cells in the population express β tubulin III. In some embodiments, greater than or equal to 60% of the cells in the population express β tubulin III. In some embodiments, 1% to 80%, 10% to 70%, 30% to 60%, or 40% to 50% of the cells in the population express β tubulin III.

In some embodiments, cells in the population express tropomyosin-related kinase receptor B (TrkB). TrkB is involved in nervous system development and enables brain-derived neurotrophic factor binding activity and brain-derived neurotrophic factor (BDNF)-activated receptor activity. In some embodiments greater than or equal to 5%, greater than or equal to 10%, greater than or equal to 20%, greater than or equal to 30%, greater than or equal to 40%, greater than or equal to 50%, greater than or equal to 60%, greater than or equal to 70%, or greater than or equal to 80% of the cells in the population express TrkB. In some embodiments, greater than or equal to 10% of the cells in the population express TrkB. In some embodiments, greater than or equal to 20% of the cells in the population express TrkB. In some embodiments, greater than or equal to 30% of the cells in the population express TrkB. In some embodiments, greater than or equal to 40% of the cells in the population express TrkB. In some embodiments, greater than or equal to 60% of the cells in the population express TrkB. In some embodiments, 5% to 80%, 10% to 70%, 20% to 50%, or 30% to 40% of the cells in the population express TrkB.

In some embodiments, cells in the population express tropomyosin-related kinase receptor C (TrkC). TrkC is acts upstream of or within several processes including neurogenesis, neuronal action potential propagation and is predicted to enable several functions, including GPI-linked ephrin receptor activity; neurotrophin (NT3) binding activity; and p53 binding activity. In some embodiments, less than 10%, less than 5%, less than 1%, less than 0.1% of the cells in the population express TrkC. In some embodiments, less than 5% of the cells in the population express TrkC. In some embodiments, less than 1% of the cells in the population express TrkC. In some embodiments, less than 0.1% of the cells in the population express TrkC. In some embodiments, 0.01% to 10%, 0.01% to 5%, or 0.01% to 1% of the cells in the population express TrkC.

In some embodiments, cells in the population express Brain specific homeobox/POU domain protein 3a (BRN3A). BRN3A enables several functions, including DNA binding activity; DNA-binding transcription activator activity and involved in nervous system development. In some embodiments, less than 10%, less than 5%, less than 1%, less than 0.1% of the cells in the population express BRN3A. In some embodiments, less than 5% of the cells in the population express BRN3A. In some embodiments, less than 1% of the cells in the population express BRN3A. In some embodiments, less than 0.1% of the cells in the population express BRN3A. In some embodiments, 0.1% to 10%, 0.1% to 5%, or 0.1% to 1% of the cells in the population express BRN3A.

In some embodiments, cells in the population express Myo7A. Mutations in MYO7A are known to play a significant role in the development of deafness and blindness. Myo7A is expressed in the nervous system, enables protein domain specific binding activity, and acts upstream of or within several processes, including organ morphogenesis. In some embodiments, less than 40%, less than 30%, less than 20%, less than 10%, less than 5%, less than 1%, less than 0.1% of the cells in the population express Myo7A. In some embodiments, less than 30% of the cells in the population express Myo7A. In some embodiments, less than 20% of the cells in the population express Myo7A. In some embodiments, less than 10% of the cells in the population express Myo7A. In some embodiments, less than 5% of the cells in the population express Myo7A. In some embodiments, less than 1% of the cells in the population express Myo7A. In some embodiments, less than 0.1% of the cells in the population express Myo7A. In some embodiments, 0.1% to 40%, 1% to 30%, 5% to 20%, or 10% to 15% of the cells in the population express Myo7A.

In some embodiments, cells in the population express stage-specific embryonic antigen (SSEA-5). Undifferentiated cells may be identified by expression of various markers including SSEA-5. In some embodiments, less than 10%, less than 5%, less than 1%, less than 0.1%, or less than 0.01% of the cells in the population express SSEA-5. In some embodiments, less than 5% of the cells in the population express SSEA-5. In some embodiments, less than 1% of the cells in the population express SSEA-5. In some embodiments, less than 0.1% of the cells in the population express SSEA-5. In some embodiments, 0.01% to 10%, 0.1% to 5%, or 0.1% to 1% of the cells in the population express SSEA-5.

In some embodiments, cells in the population express T cell receptor alpha locus (TRA-1-60). Undifferentiated cells may be identified by expression of various markers including TRA-1-60. In some embodiments, less than 10%, less than 5%, less than 1%, less than 0.1% of the cells, or less than 0.01% of the cells in the population express TRA-1-60. In some embodiments, less than 5% of the cells in the population express TRA-1-60. In some embodiments, less than 1% of the cells in the population express TRA-1-60. In some embodiments, less than 0.1% of the cells in the population express TRA-1-60. In some embodiments, 0.01% to 10%, 0.1% to 5%, or 0.1% to 1% of the cells in the population express TRA-1-60.

In some embodiments, (a) greater than or equal to 20% of the cells in the population express SOX2; (b) greater than or equal to 10% of the cells in the population express β tubulin III; (c) greater than or equal to 5% of the cells in the population express TrkB; and (d) less than or equal to 1% of the cells in the population express TRA-1-60 and/or SSEA5.

In some embodiments, (a) greater than or equal to 30% of the cells in the population express SOX2; (b) greater than or equal to 30% of the cells in the population express PAX2; (c) greater than or equal to 30% of the cells in the population express β tubulin III; (d) greater than or equal to 20% of the cells in the population express TrkB; (e) greater than or equal to 30% of the cells in the population express GluA4; (f) less than or equal to 20% of the cells in the population express Myo7A; and (d) less than or equal to 0.1% of the cells in the population express TRA-1-60 and/or SSEA5.

Following harvesting in accordance with the methods described herein, the expanded population of auditory cells can be formulated at a specific therapeutic dose (e.g., number of cells) and cryopreserved for shipping to the clinic. The ready to administer (RTA) auditory cell therapy composition can then be administered directly after thawing without further processing. Examples of media suitable for cryopreservation include but are not limited to 90% Human Serum/10% DMSO, CRYOSTOR®, CRYOSTOR® CS10 (10% DMSO), CRYOSTOR® CS5 (5% DMSO), CRYOSTOR® CS2 (2% DMSO), STEM-CELLBANKERR, PRIME XVR FREEZIS, HYPOTHERMASOLR, Trehalose, etc. In embodiments, the cryopreservation medium comprises between about 0.5% and about 50% DMSO, e.g., about 0.5%, about 1%, about 2%, about 5%, about 10%, about 15%, about 20%, about 25%, about 30%, about 40%, or about 50%. In embodiments, the cryopreservation medium comprises between about 0.5% and about 30% DMSO. In embodiments, the cryopreservation medium comprises between about 1% and about 20% DMSO.

In some embodiments, the final cell composition are cell aggregates, filtered to separate the cell aggregates from carriers, cellular debris or matrix. In other embodiments, the cells are filtered before cryopreservation, to separate the single cells from cell aggregates, carriers, cellular debris or matrix using a single-use filter, cell strainer or mesh with pore sizes of at least 40 μm, about 50 μm, about 70 μm, about 100 μm, about 60 μm. The filter can be within a closed system. In other embodiment the separation of single cells from cell aggregates, carriers, cellular debris or matrix can be done by tangential flow centrifugation. The filtration system has a capacity to safely filter single cells through the pores I amounts of 1 million cells, 10 million cells, 100 million cells, 1 billion cells, 10 billion cells, 100 billion cells, the percent viability of post-filtered cells stored in a cryopreservation medium for between about 0 to about 8 hours is at least about 75%, 80%, 85%, 90%, 91%, 92%, 93%, 94%, 95%, 96%, 97%, 98%, 99%, or 100%. The viability can be any value or subrange within the recited ranges. In other embodiments, the percent recovery of post-filtered cells stored in a cryopreservation medium for between about 0 to about 8 hours is at least about 75%, 80%, 85%, 90%, 91%, 92%, 93%, 94%, 95%, 96%, 97%, 98%, 99%, or 100%. The recovery can be any value or subrange within the recited ranges.

In alternative embodiments, cells in the final cell compositions are single cells in a suspension. For example, single cells in a composition can be generated by dissociating the aggregates described herein by any methods known in the art that result in viable single cells.

In further embodiments, the percent viability of post-filtered cells stored in a neutralization medium for between about 0 to about 8 hours followed by storage in cryopreservation medium for between about 0 to about 8 hours is at least about 75%, 80%, 85%, 90%, 91%, 92%, 93%, 94%, 95%, 96%, 97%, 98%, 99%, or 100%. In other embodiments, the percent recovery of post-filtered cells stored in a neutralization medium for between about 0 to about 8 hours followed by storage in cryopreservation medium for between about 0 to about 8 hours is at least about 70%, 75%, 80%, 85%, 90%, 91%, 92%, 93%, 94%, 95%, 96%, 97%, 98%, 99%, or 100%. The viability can be any value or subrange within the recited ranges.

In yet other embodiments, the percent viability of post-filtered cells stored in a neutralization medium for between about 0 to about 8 hours followed by storage in cryopreservation medium for between about 0 to about 8 hours, post-thawing of the cryopreserved composition, is at least about 75%, 80%, 85%, 90%, 91%, 92%, 93%, 94%, 95%, 96%, 97%, 98%, 99%, or 100%. In still other embodiments, the percent recovery of post-filtered cells stored in a neutralization medium for between about 0 to about 8 hours followed by storage in cryopreservation medium for between about 0 to about 8 hours, post-thawing of the cryopreserved composition, is at least about, 70%, 75%, 80%, 85%, 90%, 91%, 92%, 93%, 94%, 95%, 96%, 97%, 98%, 99%, or 100%. The viability can be any value or subrange within the recited ranges.

In some embodiments, the percent viability of post-filtered auditory cells stored in a neutralization medium for between about 0 to about 8 hours at room temperature is at least about, 70%, 75%, 80%, 85%, 90%, 91%, 92%, 93%, 94%, 95%, 96%, 97%, 98%, 99%, or 100%. In some embodiments, the percent viability of post-filtered auditory cells stored in a cryopreservation medium for between about 0 to about 8 hours at room temperature is at least about, 70%, 75%, 80%, 85%, 90%, 91%, 92%, 93%, 94%, 95%, 96%, 97%, 98%, 99%, or 100%. In further embodiments, the percent viability of post-filtered auditory cells stored in a neutralization solution at room temperature for between about 0 to about 8 hours followed by storage in cryopreservation medium for between about 0 to about 8 hours at room temperature is at least about 75%, 80%, 85%, 90%, 91%, 92%, 93%, 94%, 95%, 96%, 97%, 98%, 99%, or 100%. In still further embodiments, the percent recovery of post-filtered auditory cells stored in a neutralization solution at room temperature for between about 0 to about 8 hours followed by storage in cryopreservation medium for between about 0 to about 8 hours at room temperature is at least about 75%, 80%, 85%, 90%, 91%, 92%, 93%, 94%, 95%, 96%, 97%, 98%, 99%, 100%, 105%, 110%, 115%, 120%, 125%, 130%, 140%, 150%. The viability can be any value or subrange within the recited ranges.

Auditory cells formulated in cryopreservation media appropriate for post thaw ready to administer (RTA) applications may comprise auditory cells suspended in adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water. An example of this cryopreservation media is available commercially under the tradename, CryoStor® and is manufactured by BioLife Solutions, Inc. In some embodiments, auditory cells aggregates formulated in aggregate suitable cryopreservation medium such as CryoStem®, as ready to inject product, using aggregate specific delivery system such as the Sutter Xenowork system that is used for somatic cell nuclear transfer and intracytoplasmic sperm injection, and recently used for ONP spheroids (Heuer et al. 2020).

DMSO can be used as a cryoprotective agent to prevent the formation of ice crystals, which can kill cells during the cryopreservation process. In some embodiments, the cryopreservable auditory cells therapy composition comprises between about 0.1% and about 2% DMSO (v/v). In some embodiments, the RTA Auditory cells therapy composition comprises between about 1% and about 20% DMSO. In some embodiments, the RTA auditory cells therapy composition comprises about 10% DMSO. In some embodiments, the RTA auditory cells cell therapy composition comprises about 5% DMSO. The concentration can be any value or subrange within the recited ranges.

In some embodiments, auditory cell therapy compositions formulated in cryopreservation media appropriate for post thaw ready to administer (RTA) applications may comprise auditory cells suspended in cryopreservation media that does not contain DMSO. For example, RTA sensory therapeutic cell compositions may comprise auditory cells suspended in Trolox, Na⁺, K⁺, Ca²⁺, Mg²⁺, Cl⁻, H₂PO₄⁻, HEPES, lactobionate, sucrose, mannitol, glucose, dextran-40, adenosine, glutathione without DMSO (dimethyl sulfoxide, (CH₃)₂SO) or any other dipolar aprotic solvents. An example of this cryopreservation media is available commercially under the tradename, HYPOTHERMOSOL® or HYPOTHERMOSOL®-FRS and is also manufactured by BioLife Solutions, Inc. In other embodiments, auditory cells compositions formulated in cryopreservation media appropriate for post thaw ready to administer applications may comprise auditory cells suspended in Trehalose.

The RTA auditory cell therapy compositions may optionally comprise additional factors that support auditory cell engraftment, integration, survival, potency, etc. In some embodiments, the RTA auditory cell therapy composition comprises activators of function of the auditory cell preparations described herein.

In some embodiments, the RTA auditory cell therapy compositions may be formulated in a medium comprising components that decrease the molecular cell stress during freezing and thawing processes by scavenging of free radicals, pH buffering, oncotic/osmotic support and maintenance of the ionic concentration balance.

In some embodiments, auditory cell therapies formulated in cryopreservation media appropriate for post thaw ready to administer applications may comprise one or more immunosuppressive compounds. In certain embodiments, auditory cell therapies formulated in cryopreservation media appropriate for post thaw ready to administer applications may comprise one or more immunosuppressive compounds that are formulated for slow release of the one or more immunosuppressive compounds. Immunosuppressive compounds for use with the formulations described herein may belong to the following classes of immunosuppressive drugs: Glucocorticoids, Cytostatics (e.g. alkylating agent or antimetabolite), antibodies (polyclonal or monoclonal), drugs acting on immunophilins (e.g. cyclosporin, Tacrolimus or Sirolimus). Additional drugs include interferons, opioids, TNF binding proteins, mycophenolate and small biological agents. Examples of immunosuppressive drugs include: mesenchymal stem cells, anti-lymphocyte globulin (ALG) polyclonal antibody, anti-thymocyte globulin (ATG) polyclonal antibody, azathioprine, BAS ILI X IMABO (anti-I L-2Ra receptor antibody), cyclosporin (cyclosporin A), daclizumab (anti-I L-2Ra receptor antibody), everolimus, mycophenolic acid, rituximab (anti-CD20 antibody), sirolimus, tacrolimus, and/or Mycophenolate mofetil.

In embodiments, the pharmaceutical compositions can be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use. In addition to the formulations described previously, the compositions can also be formulated as a depot preparation. Such long-acting formulations can be administered by implantation (e.g., subcutaneously). Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

The nature of pharmaceutical compositions as described herein is dependent on the mode of administration and can readily be determined by one of ordinary skill in the art. The pharmaceutical compositions described herein can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, and glycerol. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, “Remington's Pharmaceutical Sciences.”

For example, a pharmaceutical composition can be formulated for administration by drops into the ear, insufflation (such as into the ear), topical, or oral administration. In another mode of administration, the pharmaceutical composition can be directly administered in situ to the cochlea of the inner ear, such as via a cannula, catheter or pump. A cannula, catheter or pump can, for example, direct the pharmaceutical composition into the cochlear luminae or the round window of the ear. In another route of administration, the pharmaceutical composition can be injected into the ear, such as into the luminae of the cochlea (e.g., the Scala media, Sc vestibuli, and Sc tympani). Injection can be, for example, through the round window of the ear or through the cochlear capsule.

Pharmaceutical compositions in accordance with the present disclosure can further comprise a pharmaceutically-acceptable carrier. In an embodiment, a pharmaceutically-acceptable carrier can comprise dimethyl sulfoxide (DMSO). In an embodiment, a pharmaceutically-acceptable carrier does not comprise dimethyl sulfoxide. As described herein, a composition can be further adapted for cryopreservation at or below −80° C. to −195° C. In embodiments, a composition can be formulated to thaw and administered directly into a subject, e.g. via injection, without additional manipulation prior to administration. In embodiments, a composition can be formulated including a cryosolution such as CRYOSTOR® 10 (CS10) as a cryopreservation media (an animal component free defined cryopreservation medium with 10% DMSO). In some embodiments, the composition is formulated in CRYOSTOR®10. In an embodiment, a composition can be filtered using a filter kit before cryopreservation, to avoid clogging during administration though narrow cannula, syringe needle or a catheter.

A pharmaceutical composition in accordance with the present disclosure can comprise from about 1 million cells per milliliter, such as about 1.5 million cells per milliliter, such as about 2 million cells per milliliter, such as about 5 million cells per milliliter, such as about 10 million cells per milliliter, such as about 20 million cells per milliliter, such as about 25 million cells per milliliter, such as about 30 million cells per milliliter, such as about 40 million cells per milliliter, such as about 60 million cells per milliliter, such as about 70 million cells per milliliter, such as about 80 million cells per milliliter, such as about 90 million cells per milliliter, such as about 100 million cells per milliliter, such as about 0.5 million cells per milliliter, such as about 0.6 million cells per milliliter, such as about 0.8 million cells per milliliter, such as about 0.9 million cells per milliliter, such as about 1 million cells per milliliter, such as about 1.5 million cells per milliliter, or such as about 50 million cells per milliliter. The number of cells can be any value or subrange within the recited ranges.

In yet another embodiment, a pharmaceutical composition in accordance with the present disclosure can have a volume ranging from about 2 microliters to about 2 milliliter, such as about 3 microliters, such as about 4 microliters, such as about 5 microliters, such as about 6 microliters, such as about 7 microliters, such as about 10 microliters, such as about 20 microliters, such as about 50 microliters, such as about 80 microliters, such as about 100 microliters, such as about 200 microliters, such as about 500 microliters, such as about 1 milliliter or such as about 2 milliliters. The volume can be any value or subrange within the recited ranges. In an embodiment, a pharmaceutical composition in accordance with the present disclosure can be in a container configured for cryopreservation or for administration to a subject in need thereof. In an embodiment, a container can be a prefilled syringe.

In an embodiment, a pharmaceutical composition in accordance with the present disclosure can be administered at a volume ranging from about 1 microliters to about 1,800 microliters, such as about 2 microliters, such as about 3 microliters, such as about 4 microliters, such as about 50 microliters, such as about 100 microliters, such as about 200 microliters, such as about 450 microliters, such as about 1800 microliters, such as about 10 microliters, such as bout 20 microliters, or such as about 40 microliters. The volume can be any value or subrange within the recited ranges.

In some embodiments, a pharmaceutical composition in accordance with the present disclosure comprises at least about 100,000 cells, at least about 200,000 cells, at least about 300,000 cells, at least about 400,000 cells, at least about 500,000 cells, at least about 600,000 cells, at least about 700,000 cells, at least about 800,000 cells, at least about 900,000 cells, at least about 1 million cells, at least about 1.5 million cells, at least about 2 million cells, at least about 2.5 million cells, at least about 3 million cells, at least about 4 million cells, at least about 5 million cells, at least about 10 million cells, at least about 20 million cells, at least about 30 million cells, at least about 40 million cells, or at least about 59 million cells. In some embodiments, the pharmaceutical composition comprises between about 50,000 cells and 50 million cells, between about 100,000 cells and 20 million cells, between about 100,000 cells and 10 million cells, between about 100,000 cells and 1 million cells, between about 500,000 cells and 10 million cells, between about 500,000 cells and 1 million cells, between about 1 million cells and 50 million cells, or between about 10 million cells and 50 million cells. In some embodiments, the pharmaceutical composition comprises between about 100,000 cells and 10 million cells. In some embodiments, the pharmaceutical composition comprises between about 100,000 cells and 1 million cells. In some embodiments, the pharmaceutical composition comprises between about 100,000 cells and 500,000 cells. In some embodiments, the pharmaceutical composition comprises between about 500,000 cells and 1 million cells.

### Kits and Articles of Manufacture

The disclosure provides kits comprising the pharmaceutical compositions described herein, and articles of manufacture such as cryovials, syringes, syringe cartridges cannula and the like.

In some embodiments, the kit comprises instructions for use.

In some embodiments, the pharmaceutical compositions described herein are pre-packaged in a dosage unit in a cryovial, cannula, syringe or syringe cartridge, that has been cryopreserved, stored at a suitable temperature (e.g. less than or equal to −80° C., or less than or equal to −140° C.), which is ready to administer to a subject after it has been thawed to a suitable temperature, such as room temperature.

Having now generally described the invention, the same will be more readily understood through reference to the following examples that are provided by way of illustration, and are not intended to be limiting of the present disclosure. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXEMPLARY EMBODIMENTS

The invention can be understood with reference to the following enumerated embodiments.

Embodiment 1. A method for obtaining a population of auditory cells derived from undifferentiated pluripotent stem cells, the method comprising:
a) obtaining a culture of pluripotent stem cells;
b) culturing the pluripotent stem cells for a first time period under culture conditions sufficient to induce differentiation of the pluripotent stem cells to non-neuronal ectoderm cells; and
c) culturing the non-neuronal ectoderm cells from b) under culture conditions sufficient to differentiate the non-neuronal ectoderm cells to auditory cells.

Embodiment 2. A method according to embodiment, 1 wherein the pluripotent stem cells are human embryonic stem cells (hESCs).

Embodiment 3. The method according to embodiment 1, wherein the pluripotent stem cells are human induced pluripotent stem cells (hiPSCs).

Embodiment 4. The method according to embodiment 1, wherein said auditory cells comprises an aggregate of auditory cells.

Embodiment 5. The method according to embodiment 1, wherein said auditory cells comprise sensory cell populations of the ear.

Embodiment 6. The method according to embodiment 5, wherein said sensory cell populations are selected from the group consisting of hair cells, supporting cells, Otic progenitor cells and sensory neuronal progenitor cells.

Embodiment 7. The method according to embodiment 6, wherein said sensory cell populations are selected from the group consisting of hair cells, expressing hair cells markers selected from Myosin7a, supporting cells, Otic progenitor cells expressing one or more of Nestin, PAX2, PAX8, GATA3 and SOX2, and one or more sensory neuronal progenitor cells expressing markers selected from Peripherin, BRN3a, FOXG1, β-Tubulin 3, TrkB, TrkC, /MafB and GluA4.

Embodiment 8. The method according to embodiment 7, wherein said sensory neuronal progenitor cells therapeutic function, that can be assessed electrophysiological recording, demonstrating increased neuronal activity upon for example administration of Glutamate to the medium.

Embodiment 9. The method according to embodiment 8, wherein the said increase in neuronal activity can be detected by intracellular calcium sensitive dye.

Embodiment 10. The method according to embodiment 1, wherein said auditory cells comprise an aggregate of sensory neuronal progenitor cells.

Embodiment 11. The method according to embodiment 1, wherein said culture of pluripotent cells of step a) are in dynamic suspension.

Embodiment 12. The method of claim 12 wherein the said dynamic suspension include a combination of hESCs, MC (microcarrier) and ECM (extracellular matrix)

Embodiment 13 The method according to embodiment 1, wherein said culturing of non-neuronal ectoderm cells of step b) are under dynamic culture conditions.

Embodiment 14. A pharmaceutical composition for administration to a subject, said composition comprising the auditory cells according to embodiment 1 and a cryopreservation media as a formulation ready to administer to a subject directly after thawing.

Embodiment 15. A method for treating an auditory hearing condition in a subject, the method comprising administering to said subject a therapeutically effective amount of the pharmaceutical composition according to embodiment 14.

Embodiment 16. The method according to embodiment 15, wherein the therapeutically effective amount of the pharmaceutical composition include 50 million AN cells/ml and 10% DMSO.

Embodiment 17. The method according to embodiment 17, wherein the pharmaceutical composition is administered to the inner ear.

Embodiment 18. The method according to embodiment 15, wherein the pharmaceutical composition is administered to the middle ear.

Embodiment 19. The method according to embodiment 15, wherein the auditory hearing condition is conductive hearing loss, sensorineural hearing loss, central hearing loss, mixed hearing loss, auditory neuropathy spectrum disorder or central auditory processing disorder.

Embodiment 20. A method of formulating a cryopreserved auditory cell composition at intermediate differentiation state, for long term storage for re-seeding for the continuation of the process directly after thawing, said method comprising: (a) suspending the auditory cells according to embodiment 1 in a cryopreservation media to form a cell suspension, (b) storing the cell suspension at a cryopreservation temperature, and (c) thawing the cryopreserved suspension for further differentiation.

Embodiment 21. A method of formulating a cryopreserved auditory cell composition for administration to a subject directly after thawing, said method comprising: (a) suspending the auditory cells according to embodiment 1 in a cryopreservation media to form a cell suspension, (b) storing the cell suspension at a cryopreservation temperature, and (c) thawing the cryopreserved suspension for administration to said subject.

Embodiment 22. A method for replacing auditory neurons in a subject in need thereof, the method comprising administering to said subject a therapeutically effective amount of the pharmaceutical composition according to embodiment 10.

Embodiment 23. A method for augmenting an existing but damaged auditory neuron population in a subject in need thereof, the method comprising administering to said subject a therapeutically effective amount of the pharmaceutical composition according to embodiment 10.

### EXAMPLES

### Example 1: General Methods for Differentiating Pluripotent Stem Cells into Auditory Cells

### General Process

The differentiation of human Embryonic Stem Cells (hESCs) to auditory neuron-like cells was based on a stepwise process in which hESCs are differentiated through the different stages of the Auditory Neurons (AN) lineage (FIG. 1A) while supplementing the cells with the relevant growth factors through each stage (FIG. 1B). The process started by harvesting the hESCs to dissociate the cells to single cells or small clumps, and seeding. Once the cells reached the desired density and lactate concentration (between 1.68-12.29 mM) differentiation was initiated.

At first, the cells were supplemented with media containing growth factors such as BMP4, SB431542, and optionally FGF2, that drive the cells towards the Non-Neural Ectoderm (NNE) lineage. This combination of GFs was used for a duration of 3-7 days and was referred to as differentiation time frame number 1 (DTF #1).

Then the cells were provided with the next combination of GFs which included LDN193189, SB431542, FGF2 and IWP-2 to drive the cells towards the Pre-Placodal Ectoderm (PPE) lineage. This combination of GFs was used for a duration of 3-7 days and was referred to as differentiation time frame number 2 (DTF #2).

Differentiation towards the early Otic Neuronal Progenitor (ONP) lineage was initiated by supplementing the cells with media containing the growth factors CHIR99021, FGF2, IGF-1 for 7 days and was referred to as differentiation time frame number 3 (DTF #3). The old medium was removed, a fresh medium with the new combination of growth factors was added.

The next stage in the auditory development was the differentiation of the late Otic Neuronal Progenitors by supplementing the cells with media containing the GFs combination of Shh, RA, EGF, FGF2 and IGF-1. The duration of this step was 7 days and was referred to as differentiation time frame number 4 (DTF #4).

The final step towards the mature auditory neurons included supplementing the cells with IGF-1, BDNF and NT-3 for 6-40 days and referred to as differentiation time frame number 5 (DTF #5) and differentiation time frame number 6 (DTF #6). At the end of each differentiation time frame (DTF) typical markers for every stage of development were assessed by Flow cytometry (FCM). Identity markers included: Otic neuronal progenitors (GATA3, PAX8, PAX2, Nestin, SOX2; Matsouka 2017), Otic neuronal progenitor connectivity (TrkB, TrkC) and neuronal function (GluA4, Neuro-D, Brn3A, V-Glut1, β-tubulin III, Peripherin, MafB, FOXG1. Cell were stained with the various identity markers listed above and the results of the markers tested at each DTF and percent of positive cells is presented in Table 5.

The cell culture throughout the entire process was either in a 2D or 3D system, and in some cases, it was beneficial to harvest the cells at any given time frame and reseed the cells to optimize the differentiation process. The data in Table 5 were generated using a 2D system.

TABLE 5
DTF# 1-4

| | % Of cells expressing the indicated biomarker | | | | | | | | | | | | |
|---|---|---|---|---|---|---|---|---|---|---|---|---|---|
| | | | | TRA-1- | | | | | | | β | | |
| | | | | 60 (−)/ | | | | | | | tubulin | TrkB | |
| | SOX2 | PAX6 | P75 | P75(+) | Nestin | PAX8 | SIX1 | PAX2 | CD133 | GATA3 | III | Surface | TrkC |
| DTF#1 | 95.90 | 79.60 | 97.20 | 31.70 | 57.70 | NA | NA | NA | NA | NA | NA | NA | NA |
| DTF#2 | 47.60 | 8.57 | 97.50 | 73.60 | 88.10 | 5.64 | NA | 17.20 | 17.60 | 56.70 | 28.80 | NA | |
| DTF#3 | 18.20 | 0.09 | 68.10 | NA | 88.00 | 1.93 | NA | 16.20 | 27.20 | 6.00 | 34.40 | NA | 0.85 |
| DTF#4 | 72.20 | NA | 30.40 | 29.40 | 96.50 | 30.30 | 13.30 | 72.00 | 52.70 | 6.04 | 55.00 | 2.16 | 0.11 |

TABLE 5
DTF #5-6

| | % Of cells expressing the indicated biomarker | | | | | | | | | | | | |
|---|---|---|---|---|---|---|---|---|---|---|---|---|---|
| | | | | | | | | β tubulin | TrkB | | | | |
| | SOX2 | Nestin | PAX8 | PAX2 | Six1 | CD133 | GATA3 | III | Surface | TrkC | GluA4 | BRNA3 | Myo7A |
| DTF#5 | 54.70 | 90.60 | 8.39 | 77.50 | 63.10 | NA | 0.19 | 20.40 | 19.00 | 0.03 | 25.70 | 0.99 | 7.43 |
| DTF#6 | 78.10 | NA | 6.76 | 91.50 | 80.70 | 95.60 | 0.55 | 11.90 | 29.50 | 0.00 | 85.30 | 1.45 | 9.97 |

Differentiation Process of H1 hESCs to ANP:

The initiation of AN differentiation began with the replacement of media containing the first differentiation time frame (DTF #1) growth factors (Table 6). The cells were cultured for 6 days (FIG. 2) in which media was replaced every day, except for day 1 where the cells were treated with double volume of media and were untouched until day 3 (due to the weekend) to reach ectodermal cells. At the end of this time frame the cells were harvested and were analyzed by fluorescence-activated cell sorting (FACS) for typical ectodermal markers expressions such as PAX6, SOX2 and NNE markers (Table 7).

### Differentiation at DTF #2:

The differentiation process continued into the next differentiation time frame (DTF #2). At this point, the cells were treated with media containing a new combination of GFs according to Table 6. The media was replaced every day except for day 8 where the cells were treated with double volume of media and were untouched until day 10 (due to the weekend) for the duration of 6 days total (FIG. 2). At the end of this time frame the cells were harvested and analyzed via FACS for Pre-Placodal Ectoderm (PPE) markers such as P75 and GATA3 (Table 7).

### Differentiation at DTF: 3:

Next, the differentiation process continued into the next differentiation time frame (DTF #3). The cells were treated with media containing a new combination of GFs according to Table 6. The media was replaced every day except for day 15 where the cells were treated with double volume of media and were untouched until day 17 due to the weekend for the duration of 7 days total (FIG. 2). At the end of this time frame the cells were harvested and analyzed via FACS for typical nonneuronal ectoderm Pre-Placodal Ectoderm (PPE) such as p75 expressing cells and Neural progenitors' markers such PAX2 and GATA3 (Table 7).

### Differentiation at DTF: 4:

At day 19, the process continued into the next differentiation time frame (DTF #4) where the cells were treated with media containing a new combination of GFs according to Table 7. The media was replaced every day except for day 22 where the cells were treated with double volume of media and were untouched until day 24 (due to the weekend) for the duration of 7 days total (FIG. 2). At the end of this time frame the cells were harvested and analyzed via FACS for typical Neural progenitors' markers such PAX2, PAX8 and more neural markers such as β tubulin III (Table 7).

### Differentiation at DTF: 5 and DTF: 6:

At the end of DTF #4 (Day 26), the cells were harvested (with TrypLE™ Select) as single cells and were seeded in flasks containing media with the GFs combination of DTF #5 (Table 6) and ROCK Inhibitor. At this stage media was replaced every 2 days (except for day 28 where the cells were treated with double volume of media and were untouched until day 31 due to the weekend) until day 35 (FIG. 2). At the end of this time frame the cells were harvested and were assessed via FACS for more Neural markers such as Nestin and PAX2 (Table 7).

The process continued by reseeding the harvested cells from the end of DTF #5 (once again as single cells) in flasks containing double volume of media with DTF #6 GFs combination (Table 6) and ROCK Inhibitor. The cells were incubated for 3 days after seeding and then the media was replaced every 2 days until day 42 (FIG. 2). At day 42 the cells were harvested and were analyzed by FACS for mature Neural markers such as TrkB and GluA4 (Table 7).

TABLE 6
GFs combinations in each DTF during AN differentiation

| | DTF, GFs supplemented | | | | | |
|---|---|---|---|---|---|---|
| | DTF#1 | DTF#2 | | DTF#4 | DTF#5 | DTF#6 |
| | BMP4, | LDN193189, | DTF#3 | Shh, RA, | IGF-1, | IGF-1, |
| | SB431542, | SB431542, | CHIR99021, | EGF, FGF2, | BDNF, NT- | BDNF, NT- |
| GFs | FGF2 | FGF2, IWP-2 | FGF2, IGF-1 | IGF-1 | 3 | 3 |
| Duration | 6 Days | 6 Days | 7 Days | 7 Days | 9 Days | 7 Days |

TABLE 7
Markers expressions during AN differentiation

| DTF#2 | |
|---|---|
| P75 (+)/ | DTF#3 |

| | DTF#1 | | | TRA-1- | % | % GATA3 | | % CD133 |
|---|---|---|---|---|---|---|---|---|
| | % SOX2 | % PAX6 | % AP2 | 60 (−) | P75 | (Neural | % P75 | (Neural |
| Group | (ectoderm) | (ectoderm) | (NNE) | (PPE) | (PPE) | (progenitor) | (PPE) | (progenitor) |
| 1.1 | 95.90 | 71.60 | 5.09 | 73.60 | 97.50 | 56.70 | 68.10 | 27.20 |

| | DTF#4 | | | | | DTF#6 | |
|---|---|---|---|---|---|---|---|
| | | | % β tubulin | DTF#5 | | | % |
| | % PAX2 | % PAX8 | III | % PAX2 | % Nestin | % TrkB | GluA4 |
| | (Neural | (Neural | (Neural | (Neural | (Neural | (Neural | (Mature |
| Group | (progenitor | (progenitor | marker) | progenitor) | progenitor) | marker) | neuron) |
| 1.1 | 72.00 | 30.30 | 55.00 | 77.50 | 90.60 | 29.50 | 85.30 |

The FACS analysis revealed the marker expressions of the cells through the different stages of the AN differentiation. At the end of DTF #1 there were high levels of SOX2 and PAX6 which indicate a successful differentiation towards the ectoderm, and some expression of AP2 that indicate the beginning of differentiation towards the NNE lineage. At the end of DTF #2 the cells expressed high levels of P75 which is a marker for the PPE lineage, and in addition most of the population that was positively stained for P75 and negatively stained for TRA-1-60 showed high commitment to the PPE lineage. At this stage the cells also expressed high levels of GATA3 which indicates the direction towards ONP cells differentiation.

At the end of DTF #3 cells had also expressed CD133 which is another marker for neural progenitors-suggesting promotion of the neural lineage. At the end of DTF #4 there were high levels of the ONP markers PAX2 and PAX8 as well as the neural marker B tubulin III. At the end of DTF #5 we could detect a slight increase in PAX2 expression and high levels of Nestin which is another marker of neural progenitors. At the end of DTF #6 the cells expressed the neural marker TrkB and high levels of GluA4 which indicates on mature auditory neurons. All together, these results suggest that the cells have gone through the different lineages towards the differentiation of AN.

### Example 2: hESC Culture Conditions and Differentiation into Auditory Cells

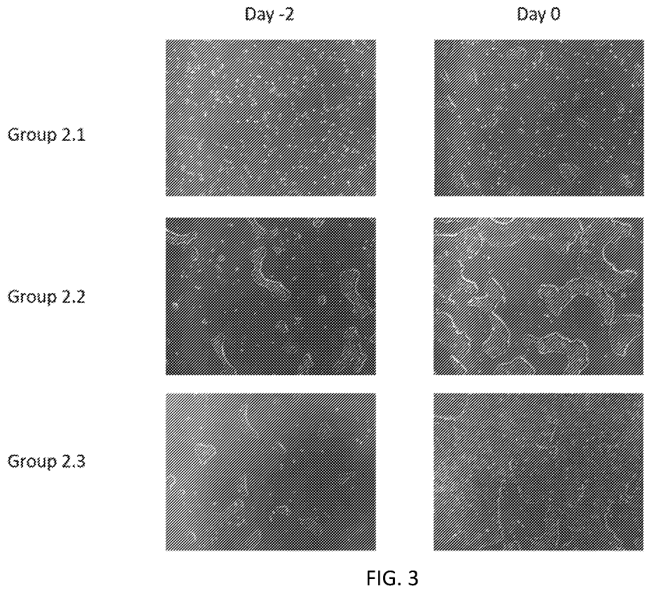

hESCs cultures in different conditions result in successful differentiation toward AN. hESCs were harvested either as small clumps or as single cells and were seeded in different densities (1,200-20,000 live cells/cm²). The cells were cultured in a monolayer environment in different vessels such as T-flasks and plates coated with iMatrix-511 E8 and were cultured for 2-5 passages until reaching the desired lactate concentrations for differentiation initiation (1.68-12.29 mM) with % confluency of 5-80% prior to differentiation initiation, according to Table 8. These parameters at the differentiation initiation result in successful differentiation towards AN cells, as FACS results show marker expressions of the NNE/PPE lineage such as AP2, P75 at the first stages of differentiation, as well as high levels of neural progenitor markers such as PAX8 and B tubulin III at the more progressive stages of the differentiation (Table 9). The morphology of pre-differentiated hESCs of the groups in Error! Not a valid bookmark self-reference.8 is presented in FIG. 3.

TABLE 8
hESCs parameters pre-AN differentiation

| | | Seeding | | |
|---|---|---|---|---|
| | | conditions | Lactate | |
| Group | cell density | (clumps/Single | concentration | % |
| # | (live cells/cm²) | cells) | (mM) | Confluence |
| 2.1 | 10,000 | Single cells | 1.68 | 5 |
| 2.2 | 3,000 | Clumps | 5.52 | 50 |
| 2.3 | 4,800 | Clumps | 12.29 | 80 |

TABLE 9
Markers of the different Groups at DTF#1/4

| | |
|---|---|
| End of DTF#1 | End of DTF#4 |

| Group | % AP2 | % 75 | % PAX8 | % β tubulin III |
|---|---|---|---|---|
| 2.1 | 3.24 | 99.60 | 49.80 | 78.70 |
| 2.2 | 5.09 | 97.20 | 30.30 | 55.00 |
| 2.3 | 5.49 | 99.50 | 24.70 | 40.30 |

### Example 3: Timing of Differentiation Time Frame 1

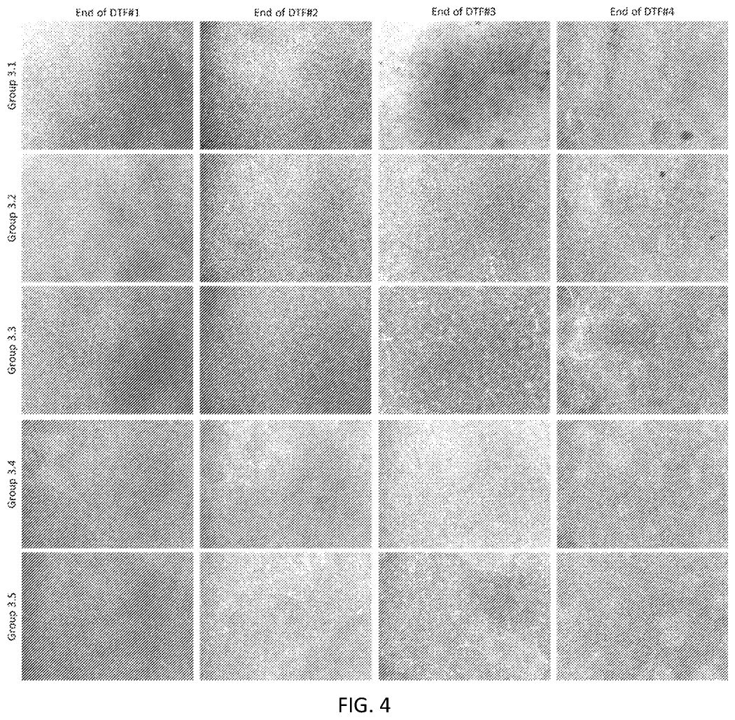

Differentiation time frame #1 (DTF #1) duration result in successful differentiation toward AN. The full process as presented in FIG. 1, involves differentiation of Non-Neuronal Ectoderm (NNE) to Pre-Placodal Ectoderm (PPE) phases. The culturing in DTF #1 was tested for different durations as presented in Table 10. Morphology assessment of DTF #1 toward AN as detailed in Table 10 is presented in FIG. 4. In addition, ANs' markers expression of cells at the end of each DTF from all groups are presented in Table 11.

TABLE 10
GFs combination during differentiation

| | GFs supplemented | | | |
|---|---|---|---|---|
| | DTF#1 | DTF#2 | | |
| | BMP4, | LDN193189, | DTF#3 | DTF#4 |
| | SB431542, | SB431542, | CHIR99021, | Shh, RA, EGF, |
| Group # | FGF2 | FGF2, IWP-2 | FGF2, IGF-1 | FGF2, IGF-1 |
| 3.1 | 3 days | 5 days | 7 days | 7 days |
| 3.2 | 4 days | | | |
| 3.3 | 5 days | | | |
| 3.4 | 6 days | | | |
| 3.5 | 7 days | | | |

TABLE 11
Markers expression of cells cultured to ANs differentiation

| | | | End of | | | | End of DTF#4 | | | |
|---|---|---|---|---|---|---|---|---|---|---|
| | End of DTF#1 | | DTF#2 | | End of DTF#3 | | Neural | | | Neuronal |
| | Ectoderm | | PPE | | Neural | ONP | progenitors | | ONP | % β |
| Group | % | % | % | % | progenitors | % | % | % | % | tubulin |
| # | SOX2 | PAX6 | AP2 | P75 | % CD133 | GATA3 | CD133 | Nestin | GATA3 | III |
| 3.1 | 96.60 | 2.04 | 11.20 | 92.20 | 29.70 | 0.36 | 33.40 | 96.80 | 7.72 | 36.70 |
| 3.2 | 97.50 | 30.70 | 10.40 | 93.00 | 23.00 | 1.52 | 35.30 | 96.60 | 7.48 | 27.90 |
| 3.3 | 89.40 | 67.30 | 4.85 | 85.30 | 20.00 | 3.05 | 43.80 | 96.90 | 15.90 | 49.30 |
| 3.4 | 89.70 | 56.10 | 6.19 | 97.30 | 15.30 | 8.43 | 19.00 | 96.80 | 10.70 | 15.60 |
| 3.5 | 56.90 | 54.90 | 6.61 | 93.80 | 22.30 | 6.24 | 22.10 | 96.80 | 14.70 | 22.20 |

At the end of DTF #1, the results show differentiation to Ectoderm by high levels of SOX2 (56-97%) and PAX6 (2-67%) markers. At the end of DTF #2, the cells expressed high levels of P75 (85-97%) which is a marker for the PPE lineage. At the end of DTF #3, cells had expressed CD133 (15-29%) which is marker for neural progenitors. At the end of DTF #4, there were high levels of Nestin (96%), the neural progenitor marker, as well as β tubulin III (22-49%), which is a neuronal marker. In addition, at the end of DTF #4 a slight increase in CD133 and GATA3 expression from end of DTF #3 was detected. All together these results suggest that the cells have gone through the different lineages towards the differentiation of ANs.

hESCs were successfully cultured to differentiation of Non-Neuronal Ectoderm (NNE) to Pre-Placodal Ectoderm (PPE) phases (DTF #1) in all days' durations (3-7 days).

### Example 4: Timing of Differentiation Time Frame #2

Differentiation time frame #2 (DTF #2) duration result in successful differentiation toward AN. The full process as presented in FIG. 1, involves differentiation of Pre-Placodal Ectoderm (PPE) to Early Otic Neuronal Progenitors (ONP) phases. The culturing in DTF #2 was tested in different duration. Examples for several days' durations are presented in Table 12.

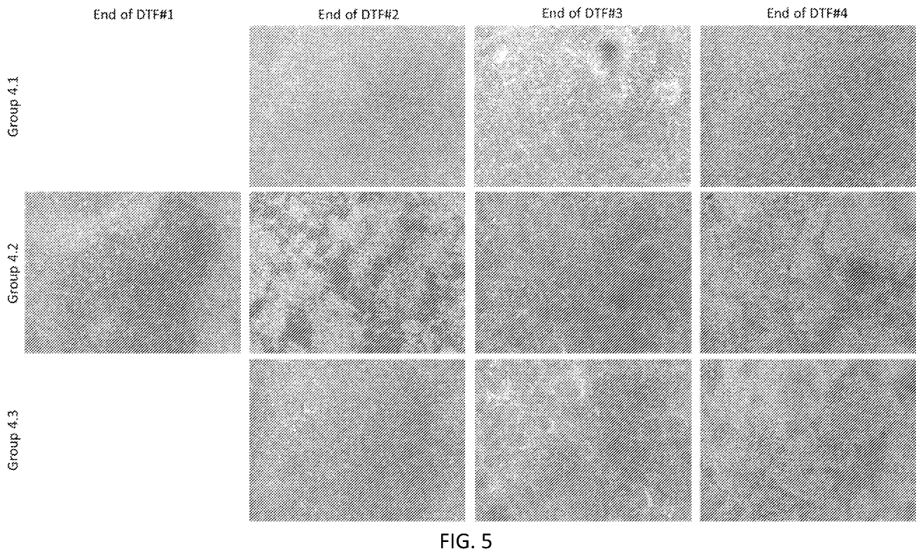

Morphology assessment of DTF #2 toward AN Table 12 is presented in FIG. 5.

In addition, AN markers expression of cells at the end of each DTF from all groups are presented in Table 13.

TABLE 12
GFs combination during differentiation

| | GFs supplemented | | | |
|---|---|---|---|---|
| | DTF#1 | DTF#2 | | DTF#4 |
| | BMP4, | LDN193189, | DTF#3 | Shh, RA, |
| | SB431542, | SB431542, | CHIR99021, | EGF, FGF2, |
| Group # | FGF2 | FGF2, IWP-2 | FGF2, IGF-1 | IGF-1 |
| 4.1 | 6 days | 5 days | 7 days | 7 days |
| 4.2 | | 6 days | | |
| 4.3 | | 7 days | | |

TABLE 13
Markers expression of cells cultured to ANs differentiation

| | | | End of DTF#2 | | | | | | | |
|---|---|---|---|---|---|---|---|---|---|---|
| | | | PPE | | | | End of DTF#4 | | | |
| | End of DTF#1 | | | % P75 | End of DTF#3 | | Neural | | Neuronal | |
| | Ectoderm | | | (+)/ | Neural | ONP | progenitors | | % β | |
| Group | % | % | % | TRA-1- | progenitors | % | % | % | tubulin | AN |
| # | SOX2 | PAX6 | P75 | 60 (−) | % CD133 | PAX8 | CD133 | Nestin | III | TrkB |
| 4.1 | 97.40 | 78.70 | 90.40 | 63.80 | 9.31 | 14.60 | 22.40 | 87.30 | 29.90 | 46.40 |
| 4.2 | | | 92.70 | 70.40 | 7.37 | 15.60 | 33.30 | 84.80 | 24.20 | 40.10 |
| 4.3 | | | 89.10 | 79.10 | 5.15 | 9.92 | 35.60 | 79.70 | 14.10 | 30.70 |

At the end of DTF #1, the results show differentiation to Ectoderm by high level of SOX2 (97%) and PAX6 (78%) markers. At the end of DTF #2, the cells expressed high levels of P75 (89-92%) which is a marker for the PPE lineage and in addition cells that were positively for P75 stained and negatively for TRA-1-60 showed high levels of expression (63-79%). At the end of DTF #3, cells had expressed CD133 (5-9%), the neural progenitor marker, and PAX8 (9-15%), which is ONP marker. At the end of DTF #4, cells had expressed B tubulin III (14-29%), the neuronal marker and TrkB (30-46%), which is a AN marker. there were high levels of Nestin (79-87%), which is marker for neural progenitors. In addition, at the end of DTF #4 we could detect a slight increase in CD133 expression from end of DTF #3. All together these results suggest that the cells have gone through the different lineages towards the differentiation of ANs.

Cells were successfully cultured to differentiation of Pre-Placodal Ectoderm (PPE) to Early Otic Neuronal Progenitors (ONP) phases (DTF #2) in all days' durations (5-7 days).

### Example 5: Growth Factor Combinations in Differentiation Time Frame #1

Different GFs combination for Differentiation time frame #1 (DTF #1). In this time frame, cells are cultured with BMP4, SB431542 and FGF2 growth factors that drive the cells towards the Ectoderm lineage. The previous example showed the duration of DTF #1 and this example shows different GFs combinations—with FGF2 and without FGF2 in DTF #1. The two different combinations are detailed in Table 14.

TABLE 14
GFs combination during differentiation

| | GFs supplemented | | | | |
|---|---|---|---|---|---|
| | DTF#1 | | DTF#2 | | DTF#4 |
| | BMP4, | | LDN193189, | DTF#3 | Shh, RA, |
| | SB431542, | BMP4, | SB431542, | CHIR99021, | EGF, FGF2, |
| Group # | FGF2 | SB431542 | FGF2, IWP-2 | FGF2, IGF-1 | IGF-1 |
| 4.1 | 6 days | NA | 6 days | 7 days | 7 days |
| 4.4 | NA | 6 days | | | |

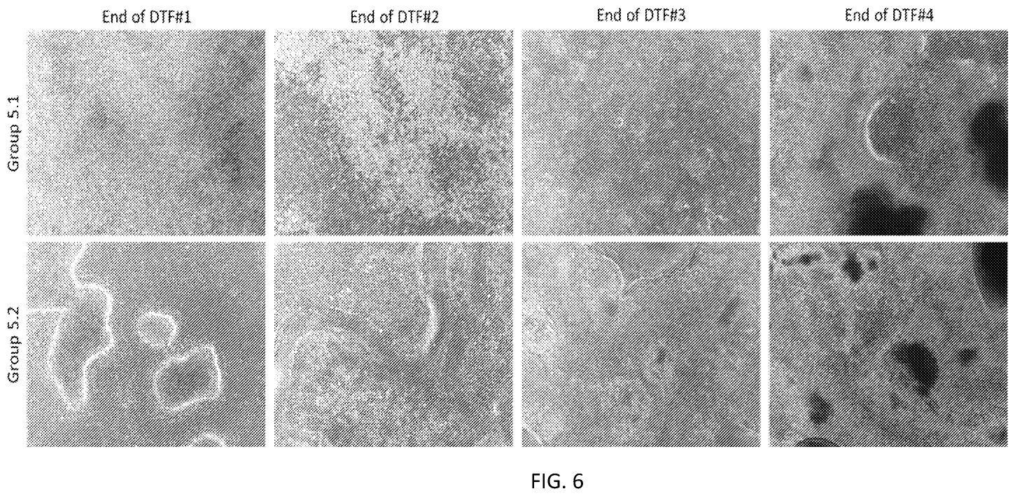

Morphology assessment of DTF #1 with and without FGF2 toward AN Table 14 is presented in FIG. 6.

In addition, ANs' markers expression of cells at the end of each DTF from all groups are presented in Table 15.

TABLE 15
Markers expression of cells cultured to ANs differentiation

| | End of | | | | | | End of DTF#4 | | | |
|---|---|---|---|---|---|---|---|---|---|---|
| | DTF#1 | | End of DTF#2 | | End of DTF#3 | | Neural | | | Neuronal |
| | Ectoderm | | PPE | | Neural | ONP | progenitors | | ONP | % β |
| | % | % | % | % P75 (+)/ | progenitors | % | % | % | % | tubulin |
| Group # | SOX2 | PAX6 | P75 | TRA-1-60 (−) | % CD133 | PAX8 | CD133 | Nestin | PAX8 | III |
| 5.1 | 95.70 | 36.30 | 99.20 | 59.40 | 33.30 | 21.10 | 70.30 | 92.10 | 54.00 | 53.90 |
| 5.2 | 84.00 | 82.40 | 90.20 | 85.20 | 41.70 | 6.88 | 44.60 | 69.20 | 58.90 | 37.30 |

At the end of DTF #1, the results show differentiation to Ectoderm by high level of SOX2 (95-84%) and PAX6 (82-36%) markers. At the end of DTF #2, the cells expressed high levels of P75 (99-90%) which is a marker for the PPE lineage and in addition cells that were positively for P75 stained and negatively for TRA-1-60 showed high levels of expression (59-85%). At the end of DTF #3, cells had expressed CD133 (33-41%), the neural progenitor marker, and PAX8 (6-21%), which is ONP marker. At the end of DTF #4, there were high levels of Nestin (69-92%), the neural progenitor marker, as well as β tubulin III (37-53%), which is a neuronal marker. In addition, at the end of DTF #4 we could detect a slight increase in CD133 and PAX8 expression from end of DTF #3. All together these results suggest that the cells have gone through the different lineages towards the differentiation of ANs.

hESCs were successfully cultured to differentiation of Non-Neuronal Ectoderm (NNE) to Pre-Placodal Ectoderm (PPE) phases (DTF #1) with two different GFs combination.

### Example 6: Differentiation Culture Systems

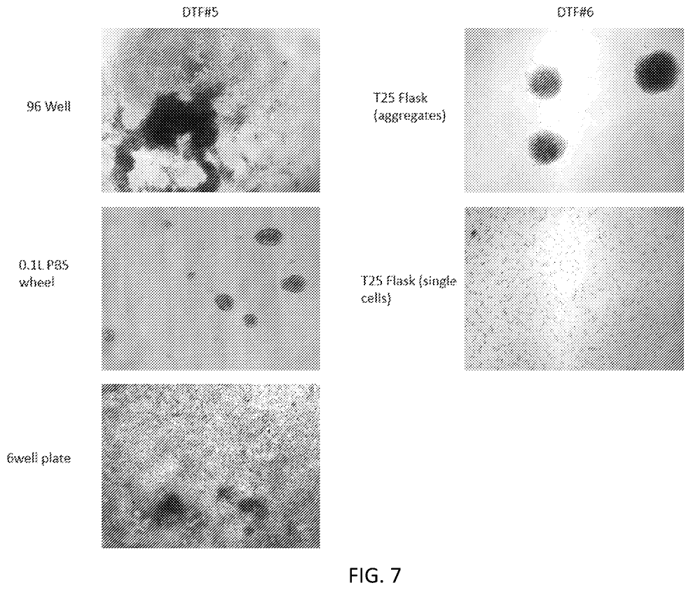

Different culturing systems for ONP maturation. During the process development, several systems were tested to optimize the stage of ONP maturation during AN differentiation (FIG. 7). Dynamic systems were used to form aggregates in 0.1 L PBS wheels while static systems were used for aggregate formation in 96w plates or monolayer single cells culturing in flasks (as described above in Example 2) and 6 well plates. Prior to using these systems, the cells were harvested (either by TrypLE™ Select or transferred as hole aggregates) at the final stages of the differentiation process (DTF #5/6) and were seeded in different densities at each culture system (Table 16).

The techniques used for ONP maturation also included the transition of aggregates from the dynamic culture of PBS wheels to the static culture of flasks-either as hole aggregates or as single cells after an enzymatic and mechanical dissociation of the aggregates. In other groups, aggregates that were cultured in 96w plates have been transferred (without dissociation) to a 0.1 L PBS wheel until the end of DTF #5, and were transferred again as hole aggregates to a T25 flask, or were harvested as single cells and reseeded in a T25 flask until the end of DTF #6.

TABLE 16
systems used for ONP maturation

| | Culture | | Cell seeding |
|---|---|---|---|
| System | Timing | Densities | conditions |
| 96 well | DTF#5 | 25,000-250,000 | aggregates |
| plate | | cells/well | |
| 0.1 L PBS | DTF#5/6 | 25,000-500,000 | aggregates |
| wheel | | cells/well | |
| T25/T75 | DTF#5/6 | 50,000 cells/well | Single cells/ |
| Flasks | | (or 10 ml of | aggregates |
| | | aggregates | |
| 6 well plate | DTF#1-6 | 2,400-3,000 | clumps |
| | | cells/well | |

### Example 7: Cryopreservation of the Intermediate Cell Bank

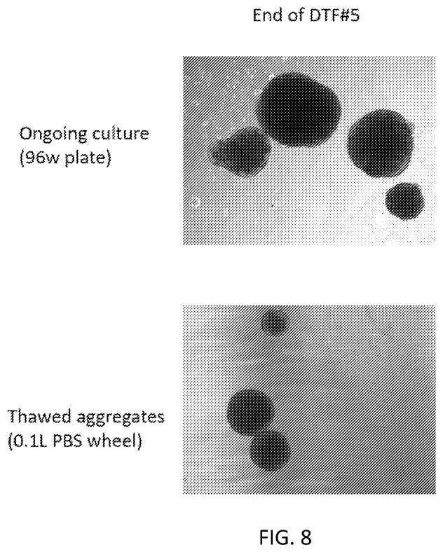

Cryopreservation of intermediate cell bank of mid-late ONPs and thawing prior to AN maturation step. At the end of DTF #4 the cells were either continued to differentiate towards the final stages of AN differentiation (DTF #5) or harvested and Cryopreserved (with CS10) to create an intermediate cell bank (ICB) of mid-late ONPs. When cultured in 96 wells, the cells were cryopreserved as aggregates and were later thawed and cultured in a 1 L PBS wheels (FIG. 8). FACS analysis shows similar marker expressions between the ongoing cultured aggregates and the thawed aggregates at the end of DTF #5 (Table 17).

TABLE 17
Marker expression of thawed and
ongoing cells at the end of DTF#5

| hESCs | |
|---|---|
| marker | Neural markers |

| Group | % SSEA-5 | % CD133 | % TrkB |
|---|---|---|---|
| Ongoing culture | 0.031 | 68.20 | 59.00 |
| Thawed aggregates | 0.088 | 74.80 | 33.20 |

FACS results show that the ongoing cultured aggregates as well as the thawed aggregates have remained with a low pluripotency which is shown by low levels of the hESCs marker SSEA-5. Both groups show similar neural marker expressions such as high levels of CD133 and low levels of GATA3, and in addition both groups express TrkB.

### Example 8: Cryopreservation of Auditory Cells

Cryopreservation of auditory neuronal progenitors (ANP) in a Thaw and Inject (TAI) formulation. Cells at differentiation days 35 were harvested and formulated in CryostorR 10% in final concentration of 50 million cell/ml, as a ready to administer (RTA) final product. 0.25 ml of the formulated cells were aliquoted into cryovials and frozen using controlled freezing protocol (CryoMed, Thermo Scientific). Cryovials were transferred to long term storage in a vapor phase N2 Tank. Vials of several batches were tested for viability and expansion potential post thawing, by seeding them for 14 days at 0.5 million cells/cm² on tissue culture plates. Culture media was replaced every 2-3 days and included BDNF, IGF1 and NT3. At day 14 cells were harvested and counted for assessing their percent of viability and yield (harvested cells/seeded cells) as detailed in Table 18. Table 18 illustrates the cell viability and yield of several AN batches after Cryopreservation (formulated as RTA formulation in CryoStor® 10% in a clinically effective cell concentration) and thawing for seeding and growing for 14 days.

TABLE 18

| | | | | Live | | | | Yield |
|---|---|---|---|---|---|---|---|---|
| | | | Viability | cells | | | Day 14 | at day |
| | Diff. | % Viability | post | post | Seeding density | Day 14% | harvest | 14 post |
| Group | Day | after harvest | thaw | thaw | cells/cm2 | viability | density | seeding |
| 8.1 | 35 | 99 | 89 | 7.77 | 500,000 | 95 | 1,120,000 | 2.2 |
| 8.2 | 35 | 99 | 89 | 4.395 | 500,000 | 97 | 1,556,314 | 3.1 |
| 8.3 | 33 | 97 | 87 | 5.94 | 500,000 | 87 | 848,485 | 1.7 |
| 8.4 | 35 | 96 | 88 | 3.51 | 500,000 | 94 | 1,487,179 | 3.0 |

The cells were viable and grew well post thawing from an RTA formulation.

### Auditory Cell Profiles

Suggested functional assays include Electrical Behavior axon connectivity and calcium influx using various configurations of single cell patch clamp, or multi electrode array that records every single cell in the whole cell population using extracellular electrodes in combination with a pharmacological agent specific to induce electric activity in the auditory neurons and calcium dependent dyes. See Table 19.

TABLE 19
Auditory Neuron Preliminary Batch Release

| Quality attribute | Targets/Test Procedure | Analysis Method |
|---|---|---|
| Viability/recovery | TBD | NC200 |
| Phenotype | β-Tubulin III/PAX2/PAX8/ | FCM |
| | TrKB/TrKC/GluA4/Nestin/ | |
| | SOX2/FoxG1/MafB/Peripherin/ | |
| | Brn3a | |
| Secretion profile | Calcium influx | FCM, FLOU-8 |
| Function- | Electrical behavior | Microelectrode |
| Electrophysiology | | Array (MEA) |
| Function- Neurite | Neurite outgrowth based on ICC | Florescent imaging |
| outgrowth | | |
| Function- synapse | ICC/MEA/Astrocytes co-culture | ICC |
| formation | | |

FCM: flow cytometry
FLOU-8: Fluo-8 Calcium Flux Assay
NC200: NucleoCounter NC-200 ™

### Auditory Cell Stage Upon Administration

The auditory cell stage, when administered to a subject, is Pluripotent stem cell derived Otic neuronal progenitors (ONP). These auditory cells are likely to survive and integrate better in aggregate form as opposed to single cell suspension. Cell can be delivered as cell aggregates or as highly dense single cell suspension that may form cell aggregate post-delivery.

### Auditory Cell Dose

The approximate dose of the pharmaceutical composition is from about 30,000 to about 100,000 auditory cells (based on the number of neurons in the Spiral Ganglion nucleus).

### Site of Injection

The site of injection of the pharmaceutical composition is the Spiral ganglion nucleus. Holes are drilled through the otic capsule for Scala tympani injection using a 30G cannula, and additional drill may be done through the bony wall of the modiolus for Modiolar injection using a 33G cannula.

### Matured Auditory Cell Stage

The auditory cell stage, upon maturation in graft/in vitro, is Spiral Ganglion nucleus Afferent Sensory Neurons (SGN).

### EQUIVALENTS

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A pharmaceutical composition comprising a population of auditory cells, wherein:
(a) greater than or equal to 20% of the cells in the population express SOX2;
(b) greater than or equal to 10% of the cells in the population express β tubulin III;
(c) greater than or equal to 5% of the cells in the population express TrkB; and
(d) less than or equal to 1% of the cells in the population express TRA-1-60 and/or SSEA5;
wherein the pharmaceutical composition comprises a pharmaceutically acceptable carrier.

2. The pharmaceutical composition of claim 1, wherein greater than or equal to 30% of the cells in the population express SOX2.

3. The pharmaceutical composition of claim 1 or 2, wherein greater than or equal to 30% of the cells in the population express β tubulin III.

4. The pharmaceutical composition of any one of claims 1-3, wherein greater than or equal to 20% of the cells in the population express TrkB.

5. The pharmaceutical composition of any one of claims 1-4, wherein less than or equal to 0.1% of the cells in the population express TRA-1-60 and/or SSEA5.

6. The pharmaceutical composition of any one of claims 1-5, wherein greater than or equal to 50% of the cells in the population express Nestin.

7. The pharmaceutical composition of any one of claims 1-6, wherein greater than or equal to 30% of the cells in the population express PAX2.

8. The pharmaceutical composition of any one of claims 1-7, wherein less than or equal to 60% of the cells in the population express PAX8.

9. The pharmaceutical composition of any one of claims 1-8, wherein greater than or equal to 10% of the cells in the population express GluA4.

10. The pharmaceutical composition of any one of claims 1-9, wherein less than or equal to 40% of the cells in the population express Myo7A.

11. The pharmaceutical composition of any one of claims 1-10, wherein:
(a) greater than or equal to 30% of the cells in the population express SOX2;
(b) greater than or equal to 30% of the cells in the population express PAX2;
(c) greater than or equal to 30% of the cells in the population express tubulin III;
(d) greater than or equal to 20% of the cells in the population express TrkB;
(e) greater than or equal to 30% of the cells in the population express GluA4;
(f) less than or equal to 20% of the cells in the population express Myo7A; and
(d) less than or equal to 0.1% of the cells in the population express TRA-1-60 and/or SSEA5.

12. The pharmaceutical composition of any one of claims 1-11, wherein greater than or equal to 50% of the cells in the population express CD133.

13. The pharmaceutical composition of any one of claims 1-12, wherein:
(a) between about 30% to 95% of the cells in the population express SOX2;
(b) between about 10% to 60% of the cells in the population express β tubulin III;
(c) between about 5% to 70% of the cells in the population express TrkB; and
(d) between 0 to about 0.1% of the cells in the population express TRA-1-60 and/or SSEA5.

14. The pharmaceutical composition of claim 13, wherein between about 30% to 95% of the cells in the population express PAX2.

15. The pharmaceutical composition of claim 13 or 14, wherein between about 5% to 95% of the cells in the population express GluA4.

16. The pharmaceutical composition of any one of claims 13-15, wherein between 0 to about 30% of cells in the population express Myo7A.

17. The pharmaceutical composition of any one of claims 1-16, wherein the population of auditory cells comprises non-neuronal ectoderm (NNE) cells, pre-placodal ectoderm (PPE) cells, early otic neuronal progenitor (ONP) cells, mid ONP cells, late ONP cells, or any combination thereof.

18. The pharmaceutical composition of any one of claims 1-16, wherein the population of auditory cells comprises sensory cell populations of the ear.

19. The pharmaceutical composition of claim 18, wherein the sensory cell populations are selected from the group consisting of hair cells, supporting cells, otic neuronal progenitor cells and sensory neuronal progenitor cells.

20. The pharmaceutical composition of any one of claims 1-19, comprising cellular aggregates, single cells, or a combination thereof.

21. The pharmaceutical composition of any one of claims 1-20, comprising a cryopreservation medium.

22. The pharmaceutical composition of any one of claims 1-21, wherein the population of auditory cells comprises at least 100,000 cells.

23. The pharmaceutical composition of any one of claims 1-22, wherein the population of auditory cells comprises between 100,000 cells and 10 million cells.

24. A method of making the pharmaceutical composition of any one of claims 1-23, comprising
a) obtaining a culture of undifferentiated pluripotent stem cells;
b) culturing the undifferentiated pluripotent stem cells under culture conditions sufficient to induce differentiation of the pluripotent stem cells to non-neuronal ectoderm cells; and
c) culturing the cells from (b) under culture conditions sufficient to differentiate the non-neuronal ectoderm cells into auditory cells.

25. A method of producing a composition comprising a population of auditory cells, the method comprising:
(a) culturing a population of undifferentiated pluripotent stem cells in a first cell culture medium comprising Bone morphogenetic protein 4 (BMP4) and 4-[4-(2H-1,3-Benzodioxol-5-yl)-5-(pyridin-2-yl)-1H-imidazol-2-yl]benzamide (SB431542) for 1-9 days under conditions sufficient to produce non-neuronal ectodermal (NNE) cells, thereby producing a population of cells comprising NNE cells;
(b) culturing the population of cells comprising NNE cells produced in step (a) in a second cell culture medium comprising SB431542, Fibroblast growth factor 2 (FGF2), and N-(6-Methyl-2-benzothiazolyl)-2-[(3,4,6,7-tetrahydro-4-oxo-3-phenylthieno[3,2-d]pyrimidin-2-yl)thio]-acetamide (IWP-2) and 4-{6-[4-(Piperazin-1-yl)phenyl]pyrazolo[1,5-a]pyrimidin-3-yl}quinoline (LDN193189) for 1-9 days under conditions sufficient to produce pre-placodal ectodermal (PPE) cells, thereby producing a population of cells comprising PPE cells;
(c) culturing the population of cells comprising PPE cells produced in step (b) in a third cell culture medium comprising 6-((2-((4-(2,4-Dichlorophenyl)-5-(4-methyl-1H-imidazol-2-yl)pyrimidin-2-yl)amino)ethyl)amino) nicotinonitrile (CHIR99021), FGF2, and Insulin-like growth factor 1 (IGF-1) for 5-9 days under conditions sufficient to produce early otic neuronal progenitor (ONP) cells, thereby producing a population of cells comprising early ONP cells;
(d) culturing the population of cells comprising early ONP cells produced in step (c) in a fourth cell culture medium comprising Sonic Hedgehog (SHH), retinoic acid (RA), Epidermal growth factor (EGF), FGF2 and IGF-1 for 5-9 days under conditions sufficient to produce mid-late ONP cells, thereby producing a population of cells comprising mid-late ONP cells;
(e) culturing the population of cells comprising mid-late ONP cells produced in step (d) in a fifth cell culture medium comprising Brain derived neurotrophic factor (BDNF), Neurotrophin-3 (NT3), and IGF-1 for 3-45 days under conditions sufficient to produce late ONP cells, thereby producing a population of cells comprising late ONP cells; and
(f) collecting the population of cells, thereby producing the composition comprising the population of auditory cells.

26. The method of claim 25, wherein the first cell culture medium comprises FGF2.

27. The method of claim 25 or 26, wherein the BMP4 is at a concentration of between about 1-25 ng/ml in the first cell culture medium.

28. The method of claim 25 or 26, wherein the BMP4 is at concentration of 10 ng/ml in the first cell culture medium.

29. The method of any one of claims 25-28, wherein the SB431542 is at a concentration of between about 0.1-10 μM in the first and/or second cell culture medium.

30. The method of any one of claims 25-28, wherein the SB431542 is at a concentration of about 1 μM in the first and/or second cell culture medium.

31. The method of any one of claims 25-30, wherein the FGF2 is at a concentration of between about 1-25 ng/ml in the first, second, third and/or fourth cell culture medium.

32. The method of any one of claims 25-30, wherein the FGF2 is at a concentration of 10 ng/mL in the first, second, third and/or fourth cell culture medium.

33. The method of any one of claims 25-32, wherein the IWP-2 is at a concentration of between about 0.5-10 μM in the second cell culture medium.

34. The method of any one of claims 25-32, wherein the IWP-2 is at a concentration of 2 μM in the second cell culture medium.

35. The method of any one of claims 25-34, wherein the LDN193189 is at a concentration of between about 20-400 nM in the second cell culture medium.

36. The method of any one of claims 25-34, wherein the LDN193189 is at a concentration of 100 nM in the second cell culture medium.

37. The method of any one of claims 25-36, wherein the CHIR99021 is at a concentration of between about 1-25 μM in the third cell culture medium.

38. The method of any one of claims 25-36, wherein the CHIR99021 is at a concentration of 6 μM in the third cell culture medium.

39. The method of any one of claims 25-38, wherein the IGF-1 is at a concentration of between about 5-100 ng/mL in the third, fourth and/or fifth cell culture medium.

40. The method of any one of claims 25-38, wherein the IGF-1 is at a concentration of 50 ng/mL in the third, fourth and/or fifth cell culture medium.

41. The method of any one of claims 25-40, wherein the SHH is at a concentration of between about 50-1000 ng/mL in the fourth cell culture medium.

42. The method of any one of claims 25-40, wherein the SHH is at a concentration of 500 ng/mL in the fourth cell culture medium.

43. The method of any one of claims 25-42, wherein the RA is at a concentration of between about 0.2-2 μM in the fourth cell culture medium.

44. The method of any one of claims 25-42, wherein the RA is at a concentration of 0.5 μM in the fourth cell culture medium.

45. The method of any one of claims 25-44, wherein the EGF is at a concentration of between about 5-100 ng/mL in the fourth cell culture medium.

46. The method of any one of claims 25-44, wherein the EGF is at a concentration of 20 ng/mL in the fourth cell culture medium.

47. The method of any one of claims 25-46, wherein the BDNF is at a concentration of between about 5-100 ng/ml in the fifth cell culture medium.

48. The method of any one of claims 25-46, wherein the BDNF is at a concentration of 10 ng/mL in the fifth cell culture medium.

49. The method of any one of claims 25-48, wherein the NT3 is at a concentration is at a concentration of between about 5 ng/mL to 100 ng/mL in the fifth cell culture medium.

50. The method of any one of claims 25-48, wherein the NT3 is at a concentration is at a concentration of 10 ng/ml in the fifth cell culture medium.

51. The method of any one of claims 25-50, wherein the undifferentiated pluripotent stem cells comprise human embryonic stem cells (hESCs) or human induced pluripotent stem cells (hiPSCs).

52. The method of any one of claims 25-50, wherein the population of auditory cells comprises NNE cells, PPE cells, early ONP cells, mid ONP cells, late ONP cells, or any combination thereof.

53. The method of any one of claims 25-51, wherein the auditory cells comprise sensory cell populations of the ear.

54. The method of claim 53, wherein said sensory cell populations are selected from the group consisting of hair cells, supporting cells, otic neuronal progenitor cells and sensory neuronal progenitor cells.

55. The method of any one of claims 25-54, wherein the population of auditory cells comprises aggregates.

56. The method of any one of claims 25-55, wherein culturing the undifferentiated pluripotent stem cells comprises dynamic culture conditions.

57. The method of any one of claims 25-56, comprising dynamic culture conditions at any one or more of steps (a)-(e).

58. The method of any one of claims 25-57, comprising, prior to step (a), seeding the undifferentiated pluripotent stem cells at a density of 1,200-20,000 live cells/cm² in a monolayer, and culturing the cells to until a lactate concentration in the cell culture medium is 1.5-12.5 mM, and a percent confluency is 5-80%. 59 The method of any one of claims 25-58, wherein the population of undifferentiated pluripotent stem cells are cultured in the first cell culture medium for 3-7 days.

60. The method of claim 58 or 59, wherein the undifferentiated pluripotent stem cells are cultured under dynamic culture conditions.

61. The method of any one of claims 25-60, wherein the population of cells comprising NNE cells are cultured in the second cell culture medium for 3-7 days.

62. The method of any one of claims 25-61, the population of cells comprising PPE cells are cultured in the third cell culture medium for 7 days.

63. The method of any one of claims 25-62, wherein the population of cells comprising early ONP cells are cultured in the fourth cell culture medium for 7 days.

64. The method of any one of claims 25-63, wherein culturing the population of cells comprising mid-late ONP cells comprises:
(i) harvesting the population of cells comprising mid-late ONP cells;
(ii) seeding the population of harvested cells in containers comprising the fifth cell culture medium;
(iii) culturing the population of seeded cells for between 7 and 35 days;
(iv) harvesting the population of cells;
(v) seeding the population of cells in containers comprising the fifth cell culture medium; and
(vi) culturing the population of cells of 7 to 30 days.

65. The method of claim 64, wherein the fifth cell culture medium comprises a ROCK Inhibitor.

66. The method of any one of claims 25-65, comprising, prior to step (e), cryopreserving the population of cells comprising mid-late ONP cells, followed by thawing and culturing in the fifth cell culture medium.

67. The method of any one of claims 25-66, further comprising cryopreserving the population of auditory cells.

68. The method of claim 67, wherein the cryopreservation comprises suspending the population of cells in a cryopreservation medium to form a cell suspension and storing the cell suspension at less than or equal to −80° C., or less than or equal to −140° C.

69. A method of treating a subject with an auditory condition, comprising administering a therapeutically amount of the composition of any one of claims 1-23 to an inner or middle ear of the subject.

70. A method treating a subject with an auditory condition, comprising administering a therapeutically effective amount of a pharmaceutical composition comprising a population of auditory cells, wherein:
(a) greater than or equal to 20% of the cells in the population express SOX2;
(b) greater than or equal to 10% of the cells in the population express β tubulin III;
(c) greater than or equal to 5% of the cells in the population express TrkB; and
(d) less than or equal to 1% of the cells in the population express TRA-1-60 and/or SSEA5;
wherein the composition is administered to the inner or middle ear of the subject.

71. The method of claim 70, wherein:
(a) greater than or equal to 30% of the cells in the population express SOX2;
(b) greater than or equal to 30% of the cells in the population express PAX2;
(c) greater than or equal to 30% of the cells in the population express β tubulin III;
(d) greater than or equal to 20% of the cells in the population express TrkB;
(e) greater than or equal to 30% of the cells in the population express GluA4;
(f) less than or equal to 20% of the cells in the population express Myo7A; and
(d) less than or equal to 0.1% of the cells in the population express TRA-1-60 and/or SSEA5.

72. The method of any one of claims 69-71, wherein the auditory condition comprises conductive hearing loss, sensorineural hearing loss, central hearing loss, mixed hearing loss, auditory neuropathy spectrum disorder, central auditory processing disorder or tinnitus.

73. The method of any one of claims 69-72, wherein the composition is administered via injection.

74. The method of claim 73, wherein the injection comprises administration a Scala tympani or modiolus of the subject.

75. The method of claim 74, wherein the injection comprising inserting a cannula through a hole in the otic capsule, or inserting a cannula through the round window.

76. The method of any one of claims 69-75, wherein the composition is cryopreserved, and the method comprises thawing the composition prior to administration.

77. The method of any one of claims 69-75, wherein between about 100K to 1 million cells are administered to the subject.

78. Use of the composition of any one of claims 1-23 for the treatment of any auditory condition in a subject.

79. Use of the composition of any one of claims 1-23 in the manufacture of a medicament for the treatment of any auditory condition in a subject.

80. A kit, comprising the composition of any one of claims 1-23.

81. The kit of claim 80, wherein the composition is formulated in a cryovial, syringe, syringe cartridge or cannula.
